(19) United States
(12) Patent Application Publication
(10) Pub. No.: US 2013/0136705 A1
(43) Pub. Date: May 30, 2013

(54) COSMETIC COMPOSITION COMPRISING AN OIL AND A POLYMER BOTH BEARING A HYDROGEN-BOND-GENERATING JOINING GROUP, AND COSMETIC TREATMENT PROCESS

(71) Applicants: Sandrine Chodorowski-Kimmes, Senlis (FR); Charlotte Feltin, Paris (FR)

(72) Inventors: Sandrine Chodorowski-Kimmes, Senlis (FR); Charlotte Feltin, Paris (FR)

(73) Assignee: L'OREAL, Paris (FR)

(21) Appl. No.: 13/700,021

(22) PCT Filed: May 13, 2011
(86) PCT No.: PCT/EP11/57783

§ 371 Date: Feb. 12, 2013

Related U.S. Application Data
(60) Provisional application No. 61/352,911, filed on Jun. 9, 2010.

(51) Int. Cl. A61K 8/84; A61K 8/49

(52) U.S. Cl. CPC A61K 8/84; A61K 8/4953
USPC 424/59; 424/63; 514/772.3; 514/788; 424/70.7; 424/70.11; 424/61

(57) ABSTRACT

The present invention relates to a cosmetic composition comprising:

a) a supramolecular oil (compound A) which can be obtained by reaction between an oil bearing at least one nucleophilic reactive function and a joining group capable of establishing hydrogen bonds, said joining group bearing a reactive function capable of reacting with the reactive function borne by the oil, and said joining group also comprising at least one unit of formula (Ia) or (Ib):

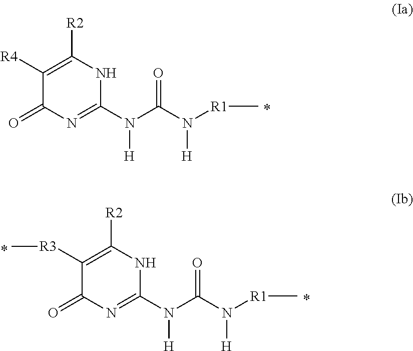

b) a polyalkene-based supramolecular polymer (compound B) which can be obtained by reaction of a functionalized polyalkene polymer with a functionalized joining group, said joining group being capable of forming at least three H (hydrogen) bonds.

The invention also relates to a cosmetic treatment process using said composition.

The present invention relates to novel cosmetic compositions comprising a mixture of H-bond-generating (supramolecular) polymers and of H-bond-generating (supramolecular) oils, and to the use thereof in the cosmetics industry, in particular in the makeup field.

Generally, when women use a makeup product, they want this product to exhibit, after application, good staying power on keratin materials, in particular the skin and/or the lips, and in particular good resistance to grease and to wear, and advantageously good transfer resistance properties.

With regard to this expectation, one or more polymers which are in fact dedicated to providing these properties of improved staying power over time is (are) commonly introduced into compositions of this type.

By way of illustration of these polymers, mention may in particular be mentioned of polyacrylates and latexes. However, the abovementioned polymers, which are advantageous in terms of staying power properties, are unfortunately capable of generating a feeling of discomfort, during application (difficult spreading, tackiness) and/or after application (tautness, mask effect), of the cosmetic product containing them.

Supramolecular polymers such as those described in applications EP 2189151 and FR 2938758 are, on the other hand, known for making it possible to obtain, on the skin, a deposit which is both comfortable and has good staying power properties. However, the deposits formed using a galenical formulation incorporating such a supramolecular polymer may have insufficient mechanical strength (which can be reflected by staining of the clothes). Moreover, bringing them into contact with fatty substances, for example a food oil in the case of a lipstick applied to the lips, can affect their integrity. Consequently, there remains a need to be free of these drawbacks with respect to supramolecular polymer-based galenical formulations.

Moreover, many cosmetic compositions exist for which properties of gloss of the deposited film, after application to the keratin materials, are desired. Mention may, for example, be made of lipsticks or nail varnishes. In order to obtain such a result, it is possible to combine particular starting materials, in particular lanolins, with “glossy” oils.

In order to improve the gloss and the staying power over time of the deposited film, it has also been proposed to use oils of triglyceride type, in the case in point castor oil, functionalized with isophorone diisocyanate (IPDI), as is described in U.S. Pat. No. 5,707,612. The functionalization with IPDI substantially improves the staying power and the gloss of castor oil; the oils thus crosslinked find an application in particular in the lipstick field.

Application EP2140858 has also proposed cosmetic compositions which make it possible to obtain a uniform, film-forming deposit on the substrate, said film allying gloss, gloss fastness and staying power of the composition, while at the same being relatively non-tacky. These effects are obtained through the use of generally solid, functionalized oils which can be obtained by reaction between an oil bearing a nucleophilic and/or electrophilic reactive function and a joining group capable of establishing hydrogen bonds with one or more partner joining groups, said joining group bearing a reactive function capable of reacting with the reactive function borne by the oil, said joining group also comprising a unit of ureidopyrimidone type. As illustrated in this application, these functionalized oils make it possible to form a cohesive and uniform film or deposit, which does not transfer to the fingers, and which is particularly glossy, said gloss being preserved over time.

However, it has been noted that the deposits obtained with these functionalized oils can be more or less tacky and exhibit a certain amount of fragility with respect to wear; moreover, it has been found that these deposits exhibit, in addition to their tacky nature, a not insignificant sensitivity to fatty substances, in particular over time.

Now, polymeric or nonpolymeric materials capable of generating deposits which exhibit good resistance, and therefore a certain amount of staying power, with respect to external attacks, in particular to “attacks” by fatty substances, such as, for example, by food oil or sebum, while at the same time preferably retaining their gloss, are sought in particular in the makeup field.

The objective of the present invention is to overcome these drawbacks and to propose a cosmetic composition which makes it possible to obtain good cosmetic properties such as good adhesion to the support (skin or hair), and therefore good staying power of the composition, optionally good gloss, while at the same time also resulting in a deposit which is not very tacky or not at all tacky, and particularly resistant to external attacks by fatty substances (oil, meal, sebum) and also to rubbing, hence the deposit being worn away to a lesser extent.

A subject of the present invention is a cosmetic composition comprising, in a cosmetically acceptable medium:

a) at least one supramolecular oil (compound A) (also referred to as supramolecular compound derived from an oil) which can be obtained by reaction between:
an oil bearing at least one nucleophilic reactive function, in particular chosen from OH and NH₂, and
a joining group capable of establishing hydrogen bonds with one or more partner joining groups, each pairing of a joining group involving at least 4 hydrogen bonds, said joining group bearing at least one reactive function capable of reacting with the reactive function borne by the oil, in particular chosen from isocyanate, acid and imidazole, said joining group also comprising at least one unit of formula (Ia)

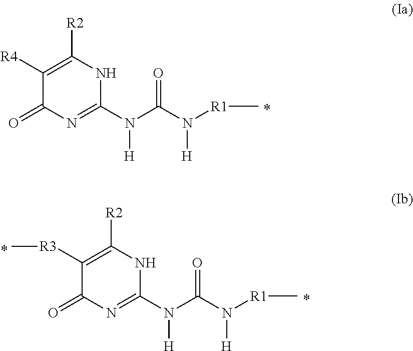

in which:
R1 and R3, which may be identical or different, represent a divalent carbon-based radical chosen from (i) a linear or branched C₁-C₃₂ alkyl group, (ii) a C₄-C₁₆ cycloalkyl group and (iii) a C₄-C₁₆ aryl group; said groups optionally comprising 1 to 8 heteroatoms chosen from O, N, S, F, Si and P; and/or being optionally substituted with an ester or amide function or with a C₁-C₁₂ alkyl radical; or a mixture of these groups;
R2 and R4, independently of one another, represent a hydrogen atom or a linear, branched or cyclic, saturated or unsaturated, optionally aromatic, C₁-C₃₂ carbon-based, in particular hydrocarbon-based (alkyl), radical which can comprise one or more heteroatoms chosen from O, N, S, F, Si and P; and
   b) at least one polyalkene-based supramolecular polymer which can result from the reaction, in particular from the condensation, of at least one polyalkene polymer functionalized with at least one reactive function, with at least one joining group functionalized with at least one reactive group capable of reacting with the reactive group(s) borne by the functionalized polyalkylene polymer, said joining group being capable of forming at least 3 H (hydrogen) bonds, preferably at least 4 H bonds, preferentially 4 H bonds.

The compositions according to the invention thus comprise 2 supramolecular compounds:
a first compound A (also known as supramolecular oil) which is obtained from an oil (preferably a nonpolymeric oil), and
a second supramolecular compound (compound B) which is a supramolecular polymer distinct from compound A.

By virtue of the invention, compositions of which the comfort is improved are obtained; they make it possible to obtain a film which is not very tacky, and which may or may not be glossy, while at the same time also being resistant to fatty substances and to mechanical wear.

Supramolecular chemistry makes it possible, through the supramolecular interactions, to mix (polymeric or nonpolymeric) compounds of different chemical natures and to obtain original properties that are different from those of the compounds used before mixing.

Thus, in the context of the present invention, it has been shown that, in certain cases, by adding a compound sensitive to wear and/or to grease (compound A) to a film-forming polymer which is itself also sensitive, it was possible to obtain, in the end, a mixture which is less sensitive than the two references alone, without modifying the other properties of the references alone, for example retaining the gloss properties of the references.

Moreover, the supramolecular polymers and the supramolecular oils used in the context of the invention are not necessarily compatible with one another and are not necessarily easily conveyed in the same range of customary cosmetic solvents, such as carbon-based oils, fatty alcohols, fatty or short esters, and most particularly in media comprising isododecane, parleam, isononyl isononanoate, octyldodecanol and/or a C₁₂-C₁₅ alkyl benzoate. Supramolecular chemistry therefore makes it possible, through the supramolecular interactions, to promote the compatibility of a polymer and of an initially incompatible oil, through the chemistry of the mixtures, and also enables conveying in the customary cosmetic solvents. The objective of this is to improve their use in the cosmetics field, in particular in the makeup field.

The compositions according to the invention thus comprise a supramolecular compound (compound A also known as supramolecular) derived from an oil which can be referred to as “H-bond-generating oil” and which can be obtained by reaction between:
an oil bearing at least one nucleophilic reactive function, in particular chosen from OH and NH₂, and
a joining group capable of establishing hydrogen bonds with one or more partner joining groups, each pairing of a joining group involving at least 4 hydrogen bonds, said joining group bearing at least one reactive function capable of reacting with the reactive function borne by the oil, in particular chosen from isocyanate, acid and imidazole, said joining group also comprising at least one unit of formula (Ia) or (Ib):

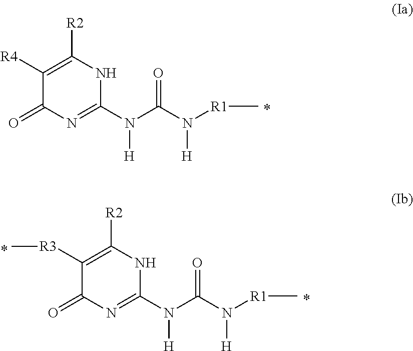

in which:
R1 and R3, which may be identical or different, represent a divalent carbon-based radical chosen from (i) a linear or branched C₁-C₃₂ alkyl group, (ii) a C₄-C₁₆ cycloalkyl group and (iii) a C₄-C₁₆ aryl group; said groups optionally comprising 1 to 8 heteroatoms chosen from O, N, S, F, Si and P; and/or being optionally substituted with an ester or amide function or with a C₁-C₁₂ alkyl radical; or a mixture of these groups;
R2 and R4, independently of one another, represent a hydrogen atom or a linear, branched or cyclic, saturated or unsaturated, optionally aromatic, C₁-C₃₂ carbon-based, in particular hydrocarbon-based (alkyl), radical which can comprise one or more heteroatoms chosen from O, N, S, F, Si and P.

In the end, the compounds A according to the invention thus comprise at least one part (HB) originating from the oil and at least one part (G) originating from the joining group, said part (G) comprising at least one unit of formula (Ia) or (Ib). In particular, said parts (HB) and (G) are connected via a covalent bond, and in particular can be connected via a covalent bond formed during the reaction between the OH and/or NH₂ reactive functions borne by the oil and the isocyanate functions borne by the joining group; or else between the NH₂ reactive functions borne by the oil and the isocyanate, acid or imidazole functions borne by the joining group. The preferential obtaining of the compounds A according to the invention can therefore in particular be represented schematically by the chemical reaction between the following entities:

(HB)—(OH)ₘ(NH₂)ₙ+(G)-(NCO)ₚ or else

(HB)—(OH)ₘ(NH₂)ₙ+(G)-(acid)ₚ or else

(HB)—(OH)ₘ(NH₂)ₙ+(G)-(imidazole)ₚ with m, n and p being non-zero integers.

The oil which can be used to prepare the compound according to the invention, which can preferably be represented schematically as (HB)—(OH)ₘ(NH₂)ₙ, is a fatty substance, or a mixture of fatty substances, which is non-crystalline at 25° C., preferably liquid at ambient temperature and at atmospheric pressure (25° C., 1 atm.), preferably apolar, or even preferably water-insoluble.

Preferably, the oil which can be used to prepare the supramolecular compound according to the invention is nonpolymeric.

The term “liquid” is intended to mean that the viscosity of the compound is less than or equal to 2500 centipoises, at 110° C., 1 atm., measured with a Brookfield DV-I or Brookfield Cap 1000+ rheometer, those skilled in the art choosing the instrument suitable for measuring viscosity.

The term “apolar” is intended to mean a compound of which the HLB (hydrophiliclipophilic balance) value is low, in particular less than or equal to 8, preferably less than or equal to 4, and even better still less than or equal to 2; preferentially, the HLB value should be sufficiently low to make it possible to obtain a supramolecular material which is not, or not too, hygroscopic.

The term “insoluble” is intended to mean that the fraction of oil which can dissolve in water, at 25° C., 1 atm, is less than 5% by weight (i.e. 5 g of oil in 100 ml of water), preferably less than 3%.

The term “fatty substance” is intended to mean, in particular but not exclusively, a hydrocarbon-based compound comprising one or more linear, cyclic or branched, saturated or unsaturated, alkyl chains containing at least 6 carbon atoms and able to comprise polar groups, for instance an acid group, a hydroxyl or a polyol, amine, amide, phosphoric acid, phosphate, ester, ether, urea, carbamate, thiol, thioether or thioester group, it being possible for this chain to contain up to 100 carbon atoms.

Preferably, the oil which can be used to prepare compound A according to the invention is a glossy oil, i.e. an oil having a refractive index of greater than or equal to 1.46 at 25° C., in particular between 1.46 and 1.55 (the refractive index being defined relative to the D line of sodium, at 25° C.).

Preferably, the oil which can be used to prepare the compound according to the invention is a non-volatile oil. The term “non-volatile oil” is intended to mean an oil capable of remaining on keratin materials, at ambient temperature and atmospheric pressure, for at least several hours, and in particular having a vapour pressure of less than 10⁻³ mmHg (0.13 Pa).

Preferably, the oil has a molar mass (Mw) of between 150 and 6000, in particular between 170 and 4000, or even between 180 and 2000, preferentially between 200 and 1500, and even better still between 220 and 800 g/mol.

The oil which can be used in the context of the present invention to prepare the supramolecular compound A used in the compositions according to the invention bears at least one reactive function capable of reacting with the reactive function borne by the joining group, in particular capable of reacting chemically with the isocyanate, acid or imidazole groups borne by the joining group; preferably, this function is an OH or NH₂ function. Preferably, the oil comprises only OH functions, in particular 1 to 3 OH functions, preferentially primary or secondary OH functions. Preferably, they are solely primary OH functions.

The oil according to the present invention is preferably a carbon-based, in particular hydrocarbon-based, oil which, in addition to the reactive function capable of reacting with the joining group, may comprise oxygen, nitrogen, sulphur and/or phosphorus atoms. The oil is very preferentially chosen from cosmetically acceptable oils.

The oil which can be used in the context of the present invention to prepare the supramolecular compound A used in the compositions according to the invention may be chosen from:

(i) linear, branched or cyclic, saturated or unsaturated, fatty alcohols comprising 6 to 50 carbon atoms and comprising one or more OH, optionally comprising one or more NH₂.

Mention may in particular be made of:
linear or branched, saturated or unsaturated, C₆-C₅₀, especially C₆-C₃₂, in particular C₈-C₂₈, monoalcohols, and in particular isostearyl alcohol, cetyl alcohol, oleyl alcohol, isopalmitoyl alcohol, lauryl alcohol, myristyl alcohol, 2-butyloctanol, 2-hexyldecanol, 2-octyldecanol, 2-octyldodecanol, 2-octyltetradecanol, 2-decyltetradecanol, 2-dodecylhexadecanol, and in particular the alcohols sold under the name Jarcol by the company Jarchem Industries, such as Jarcol I-12, Jarcol I-16, Jarcol I-20 and Jarcol I-24;
linear or branched, saturated or unsaturated, C₆-C₅₀, especially C₆-C₄₀, in particular C₈-C₃₈, and especially branched C₃₂-C₃₆, diols, and in particular the commercial product Pripol 2033 from Uniqema;
linear or branched, saturated or unsaturated, C₆-C₅₀, especially C₆-C₃₂, in particular C₈-C₂₈, triols, and in particular phytantriol;
   (ii) esters and ethers bearing at least one free OH group, and in particular partial esters and ethers of a polyol, and hydroxylated carboxylic acid esters.

The term “partial ester of a polyol” is intended to mean the esters prepared by esterification of a polyol with a carboxylic acid, which may be substituted or unsubstituted, the reaction not being complete, i.e. not carried out on all the free OH groups of the polyol; in the end, the ester thus also comprises at least one free OH. Preferably, the carboxylic acid is a monoacid. A mixture of carboxylic acids, in particular monocarboxylic acids, may also be used.

The term “partial ether of a polyol” is intended to mean the ethers prepared by etherification of a polyol, on itself or with at least one other alcohol, which is mono- or polyhydroxylated, preferably monoalcohol, the etherification reaction not being complete, i.e. not carried out on all the free OH groups of the polyol; in the end, the ether also comprises at least one free OH.

The term “hydroxylated carboxylic acid ester” is intended to mean the (mono and poly) esters prepared by reaction between a carboxylic acid bearing at least one OH function, and one or more (mono or poly) alcohols, preferably monoalcohol, it being possible for the reaction to be complete or partial (carried out on all or some of the free OH groups of the alcohol).

Among the polyols which can be used to prepare the esters or ethers above, mention may be made of propylene glycol, glycerol, neopentyl glycol, trimethylolpropane, trimethylolethane, polyglycerols, and in particular polyglycerol-2, polyglycerol-3 and polyglycerol-10; erythritol, dipentaerythritol, pentaerythritol, bis(trimethylolpropane), phytantriol, sucrose, glucose, methylglucose, sorbitol, fructose, xylose, mannitol or glucosamine; and also diol dimmers, in particular obtained from fatty acid dimers, in particular branched aliphatic and/or alicyclic C₃₂-C₃₈ and in particular C₃₆ diols, such as those defined in the article Hofer et al. European Coating Journal (March 2000), pages 26-37; and mixtures thereof.

Among the monoalcohols that can be used to prepare the esters or ethers above, mention may be made of linear or branched, preferably branched, C₃-C₅₀ alcohols, and in particular 2-ethylhexanol, octanol, isostearyl alcohol, and mixtures thereof. Among the carboxylic acids that can be used to prepare the esters or ethers above, mention may be made of linear or branched, saturated or unsaturated monoacids containing 6 to 50 carbon atoms and diacids containing from 3 to 12 carbon atoms, among which mention may be made of octyldodecanoic acid, hexyldecanoic acid, ethylhexanoic acid, isostearic acid, nonanoic acid, isononanoic acid, arachidic acid, stearic acid, palmitic acid, oleic acid, oxalic acid, adipic acid, succinic acid, fumaric acid, maleic acid, capric acid, hexanedioic acid and decanoic acid, and mixtures thereof.

Among the hydroxylated carboxylic acids that can be used to prepare the esters or ethers above, mention may be made of monohydroxylated or polyhydroxylated acids, preferably monohydroxylated acids, containing, for example, 4 to 28 carbon atoms, and in particular 12-hydroxystearic acid, ricinoleic acid, malic acid, lactic acid and citric acid, and mixtures thereof.

Thus, the oil which can be used in the present invention to prepare the supramolecular compound A may be chosen, alone or as a mixture, from:
pentaerythritol partial esters, and in particular pentaerythrityl adipate, pentaerythrityl caprate, pentaerythrityl succinate, pentaerythrityl tetraisononanoate, pentaerythrityl triisononanoate, pentaerythrityl tetraisostearate, pentaerythrityl triisostearate, pentaerythrityl 2-(tetradecyl)tetradecanoate, pentaerythrityl (tetraethyl)hexanoate and pentaerythrityl (tetraoctyl)dodecanoate;
dipentaerythritol diesters, triesters, tetraesters or pentaesters, and in particular dipentaerythrityl pentaisononanoate, dipentaerythrityl pentaisostearate, dipentaerythrityl tetraisostearate and dipentaerythrityl tris(polyhydroxystearate);
trimethylolpropane monoesters and diesters, for instance trimethylolpropane monoisostearate, trimethylolpropane diisostearate, trimethylolpropane mono(2-ethylhexylate) and trimethylolpropane di(2-ethylhexylate);
bis(trimethylolpropane) monoesters, diesters and triesters, for instance bis(trimethylolpropane) diisostearate, bis(trimethylolpropane) triisostearate and bis(trimethylolpropane) triethylhexanoate;
partial monoesters or polyesters of glycerol or of polyglycerols, and in particular:
glyceryl diisostearate, glyceryl diisononanoate,
polyglycerol-2 monoesters, diesters and triesters; for example with isostearic acid, 2-ethylhexanoic acid and/or isononanoic acid; and in particular polyglyceryl-2 isostearate; polyglyceryl-2 diisostearate; polyglyceryl-2 triisostearate; polyglyceryl-2 nonaisostearate; polyglyceryl-2 nonanoate;
polyglycerol-3 monoesters, diesters, triesters and tetraesters; for example with either isostearic acid, 2-ethylhexanoic acid and/or isononanoic acid; and in particular polyglyceryl-3 isostearate, polyglyceryl-3 diisostearate; polyglyceryl-3 triisostearate; polyglyceryl-3 nonaisostearate; polyglyceryl-3 nonanoate;
polyglycerol-10 partial esters, and in particular polyglyceryl-10 nonaisostearate; polyglyceryl-10 nonanoate; polyglyceryl-10 isostearate, polyglyceryl-10 diisostearate, polyglyceryl-10 triisostearate;
propylene glycol monoesters, for instance propylene glycol monoisostearate, propylene glycol neopentanoate, propylene glycol monooctanoate;
diol dimer monoesters, for instance isostearyl dimer dilinoleate and octyldodecyl dimer dilinoleate;
glycerol ethers, such as polyglyceryl-2 oleyl ether, polyglyceryl-3 cetyl ether, polyglyceryl-3 decyl tetradecyl ether and polyglyceryl-2 stearyl ether;
esters between a hydroxylated monocarboxylic, dicarboxylic or tricarboxylic acid and monoalcohols, and in particular:
   esters, in particular monoesters, of 12-hydroxystearic acid, such as octyl hydroxystearate and 2-octyldodecyl hydroxystearate; mention may also be made of the corresponding oligomeric polyhydroxystearates, in particular having a degree of polymerization of from 1 to 10, bearing at least one residual OH;
   lactic acid esters, and in particular C₄-C₄₀ alkyl lactates, such as 2-ethylhexyl lactate, diisostearyl lactate, isostearyl lactate, isononyl lactate or 2-octyldodecyl lactate;
   malic acid esters, and in particular C₄-C₄₀ alkyl malates, such as 2-diethylhexyl malate, diisostearyl malate or 2-dioctyldodecyl malate;
   citric acid esters, and in particular C₄-C₄₀ alkyl citrates, such as triisostearyl citrate, triisocetyl citrate and triisoarachidyl citrate;
      (iii) hydroxylated natural and modified natural plant oils, and in particular:
triglyceryl esters bearing one or more OH,
hydrogenated or nonhydrogenated castor oil, and also derivatives thereof derived in particular from the transesterification of castor oil; for instance the products Polycin M-365 or Polycin 2525 sold by Vertellus;
modified epoxidized oils, the modification consisting in opening the epoxy function to obtain a diol, and in particular hydroxylated modified soybean oil; hydroxylated soybean oils (directly hydroxylated or epoxidized beforehand); and in particular the oils Agrol 2.0, Agrol 3.0 or Agrol 7.0 sold by BioBased Technologies, LLC; the oil Soyol R2-052 from the company Urethane Soy System; the Renuva oils sold by Dow Chemical; the BioH Polyol 210 and 500 oils sold by Cargill.

According to a first particularly preferred embodiment, the oil which can be used to prepare the supramolecular compound A in the present invention is preferably chosen from:

(i) linear, branched or cyclic, saturated or unsaturated fatty alcohols comprising 6 to 50 carbon atoms, comprising one or more OH; optionally comprising one or more NH₂; and/or
(ii) esters between a hydroxylated monocarboxylic, dicarboxylic or tricarboxylic acid and monoalcohols, and in particular:
esters, in particular monoesters, of 12-hydroxystearic acid; such as octyl hydroxystearate and 2-octyldodecyl hydroxystearate; mention may also be made of the corresponding oligomeric polyhydroxystearates, in particular having a degree of polymerization of from 1 to 10, bearing at least one residual OH;
lactic acid esters, and in particular C₄-C₄₀ alkyl lactates, such as 2-ethylhexyl lactate, diisostearyl lactate, isostearyl lactate, isononyl lactate or 2-octyldodecyl lactate;
malic acid esters, and in particular C₄-C₄₀ alkyl malates, such as 2-diethylhexyl malate, diisostearyl malate or 2-dioctyldodecyl malate;
citric acid esters, and in particular C₄-C₄₀ alkyl citrates, such as triisostearyl citrate, triisocetyl citrate and triisoarachidyl citrate;
   (iii) hydroxylated natural and modified natural plant oils.

In particular, the fatty alcohols (i) may be:
linear or branched, saturated or unsaturated C₆-C₅₀, especially C₆-C₃₂, in particular C₈-C₂₈, monoalcohols, and in particular isostearyl alcohol, cetyl alcohol, oleyl alcohol, isopalmitoyl alcohol, 2-butyloctanol, 2-hexyldecanol, 2-octyldecanol, 2-octyldodecanol, 2-octyltetradecanol, 2-decyltetradecanol, 2-dodecylhexadecanol, and in particular the alcohols sold under the name Jarcol by the company Jarchem Industries, such as Jarcol I-12, Jarcol I-16, Jarcol I-20 and Jarcol I-24;
linear or branched, saturated or unsaturated, C₆-C₅₀, especially C₆-C₄₀, in particular C₈-C₃₈, and especially branched C₃₂-C₃₆, diols, and in particular the commercial product Pripol 2033 from Uniqema;
linear or branched, saturated or unsaturated C₆-C₅₀, especially C₆-C₃₂, in particular C₈-C₃₈, triols, and in particular phytantriol.

Thus, according to one preferred embodiment, the oil which can be used to prepare the supramolecular compound A in the context of the present invention is preferably chosen from linear or branched, saturated or unsaturated, C₆-C₅₀, especially C₆-C₃₂, in particular C₈-C₂₈, monoalcohols, and in particular isostearyl alcohol, cetyl alcohol, oleyl alcohol, isopalmitoyl alcohol, 2-butyloctanol, 2-hexyldecanol, 2-octyldecanol, 2-octyldodecanol, 2-octyltetradecanol, 2-decyltetradecanol, 2-dodecylhexadecanol, and in particular the alcohols sold under the name Jarcol by the company Jarchem Industries, such as Jarcol I-12, Jarcol I-16, Jarcol I-20 and Jarcol I-24.

Preferentially, the oil is chosen from:
linear or branched, saturated or unsaturated C₆-C₅₀, especially C₆-C₃₂, in particular C₈-C₂₈, monoalcohols, and in particular isostearyl alcohol, cetyl alcohol, oleyl alcohol, isopalmitoyl alcohol, lauryl alcohol, 2-butyloctanol, 2-hexyldecanol, 2-octyldecanol, 2-octyldodecanol, 2-octyltetradecanol, 2-decyltetradecanol, 2-dodecylhexadecanol; and/or
esters between a hydroxylated dicarboxylic acid and monoalcohols, and in particular malic acid, and especially C₄-C₄₀ alkyl malates, such as 2-diethylhexyl malate, diisostearyl malate or 2-dioctyldodecyl malate;
castor oil.

In particular, if it is desired to use glossy oils, the following glossy oils, for which the refractive index at 25° C. is indicated between parentheses, may be used: polyglyceryl-3 diisostearate (1.472), phytantriol (1.467), castor oil (1.475), 2-octyldodecanol (1.46), oleyl alcohol (1.461), octyl hydroxystearate (1.46), polyglyceryl-2 isostearate (1.468), polyglyceryl-2 diisostearate (1.464), diisostearyl malate (1.462), 2-butyloctanol, 2-hexyldecanol (1.45), 2-decyltetradecanol (1.457), and also mixtures thereof.

Preferably, the oils that can be used in the present invention are chosen from 2-octyldodecanol, diisostearyl malate, 2-butyloctanol, 2-hexyldecanol, 2-decyltetradecanol; hydrogenated or nonhydrogenated castor oil, and also derivatives thereof; hydroxylated modified soybean oil, and mixtures thereof.

### Joining Group for the Preparation of the Supramolecular Compound According to the Invention

The joining group which can be used to form compound A according to the invention bears at least one reactive group, in particular isocyanate or imidazole (preferably isocyanate), capable of reacting with the reactive functions, in particular OH and/or NH₂ (exclusively NH₂ for imidazole), of the oil, so as to form a covalent bond, in particular of urethane type, between said oil and said joining group.

Said joining group is capable of establishing H bonds with one or more partner joining groups, of identical or different chemical nature, each pairing of a joining group involving at least 3 H (hydrogen) bonds, preferably at least 4 H bonds, preferentially 4 H bonds.

For the purpose of the invention, the term “joining group” is intended to mean any functional group comprising groups which donate or accept H bonds, and capable of establishing at least three H bonds, preferably at least 4 H bonds, preferentially 4 H bonds, with an identical or different partner joining group.

For the purpose of the invention, the term “partner joining group” is intended to mean any joining group which can establish H bonds with one or more joining groups of the same or of another polymer according to the invention. The joining groups may be of identical or different chemical nature. If they are identical, they can then establish H bonds between one another and are then referred to as self-complementary joining groups. If they are different, they are chosen such that they are complementary with respect to H interactions.

Said joining group, bearing isocyanate groups, can thus be represented schematically as (G)-(NCO)ₚ, p being a non-zero integer, preferably equal to 1 or 2.

The joining group comprises, moreover, at least one monovalent unit of formula (Ia) and/or at least one divalent unit of formula (Ib):

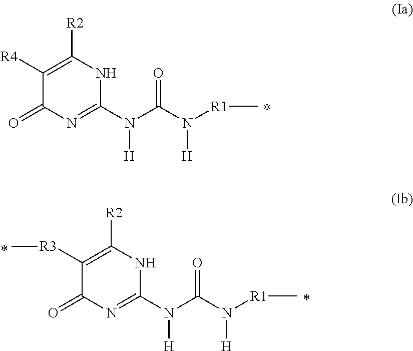

in which:
R1 and R3, which may be identical or different, represent a divalent carbon-based radical chosen from (i) a linear or branched C₁-C₃₂ alkyl group, (ii) a C₄-C₁₆ cycloalkyl group and (iii) a C₄-C₁₆ aryl group; said groups optionally comprising 1 to 8 heteroatoms chosen from O, N, S, F, Si and P; and/or said groups being optionally substituted with an ester or amide function or with a C₁-C₁₂ alkyl radical; or a mixture of these groups;
R2 and R4, independently of one another, represent a hydrogen atom or a linear, branched or cyclic, saturated or unsaturated, optionally aromatic, C₁-C₃₂ carbon-based, in particular hydrocarbon-based (alkyl) radical which can comprise one or more heteroatoms chosen from O, N, S, F, Si and P.

Preferably, the joining group comprises at least one monovalent unit of formula (Ia).

The R1 radical may in particular be:
a linear or branched, C₂-C₁₂ divalent alkylene group, in particular a 1,2-ethylene, 1,6-hexylene, 1,4-butylene, 1,6-(2,4,4-trimethylhexylene), 1,4-(4-methylpentylene), 1,5-(5-methylhexylene), 1,6-(6-methylheptylene), 1,5-(2,2,5-trimethylhexylene) or 1,7-(3,7-dimethyloctylene) group;
a C₄-C₁₂ divalent cycloalkylene or arylene group, in particular chosen from the following radicals: -isophorone-, tolylene, 2-methyl-1,3-phenylene, 4-methyl-1,3-phenylene, 4,4′-methylenebiscyclohexylene, 4,4-bisphenylenemethylene; or having the structure:

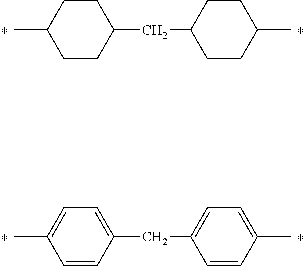

The term “-isophorone-” is intended to mean the divalent radical having the structure:

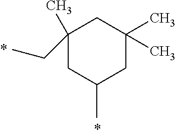

Preferentially, R1 represents -isophorone-, —(CH₂)₆— or 4,4′-methylenebiscyclohexylene.

In particular, the R2 or R4 radicals, independently of one another, may be H or else:
a C₁-C₃₂, in particular C₁-C₁₆, or even C₁-C₁₀, alkyl group
a C₄-C₁₂ cycloalkyl group;
a C₄-C₁₂ aryl group;
a (C₄-C₁₂)aryl(C₁-C₁₈)alkyl group;
a C₁-C₄ alkoxy group;
an arylalkoxy group, in particular a (C₁-C₄) arylalkoxy group;
a C₄-C₁₂ heterocycle;
   or a combination of these radicals, which may optionally be substituted with an amino, ester and/or hydroxyl function.

Preferably, R2 represents H, CH₃, ethyl, C₁₃H₂₇, C₇H₁₅, phenyl, isopropyl, isobutyl, n-butyl, tert-butyl, n-propyl, or else —CH(C₂H₅)(C₄H₉).

Preferably, R4=H.

Preferably, R3 represents a divalent radical —R′3-O—C(O)—NH—R′4- in which R′3 and R′4, which may be identical or different, represent a divalent carbon-based radical chosen from a linear or branched C₁-C₃₂ alkyl group or a C₄-C₁₆ cycloalkyl group or a C₄-C₁₆ aryl group; or a mixture thereof.

In particular, R′3 and R′4 may represent methylene, 1,2-ethylene, 1,6-hexylene, 1,4-butylene, 1,6-(2,4,4-trimethylhexylene), 1,4-(4-methylpentylene), 1,5-(5-methylhexylene), 1,6-(6-methylheptylene), 1,5-(2,2,5-trimethylhexylene), 1,7-(3,7-dimethyloctylene), 4,4′-methylenebiscyclohexylene, 2-methyl-1,3-phenylene, 4-methyl-1,3-phenylene, 4,4′-bisphenylenemethylene, 1,2-tolylene, 1,4-tolylene, 2,4-tolylene, 2,6-tolylene, 1,5-naphthylene, tetramethylxylylene or isophorone.

Most particularly, R′3 may represent a C₁-C₄ alkylene, in particular 1,2-ethylene.

Preferably, R′4 may represent the divalent radical derived from isophorone.

Most particularly, R3 may have the structure:

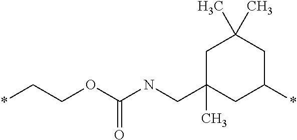 [SMILES: *CCOC(=O)NCC1(C)CC(*)CC(C)(C)C1]

Particularly preferably, in formula (Ia), it is possible to have:
R1=-isophorone-, R2=methyl and R4=H, which results in the unit of formula:

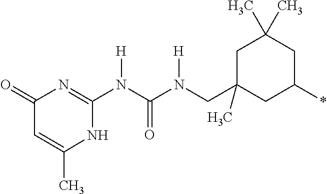

R1=—(CH₂)₆—, R2=methyl and R4=H, which results in the unit of formula:

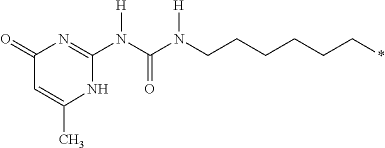

R2=—(CH₂)₆—, R2=isopropyl and R4=H, which results in the unit of formula:

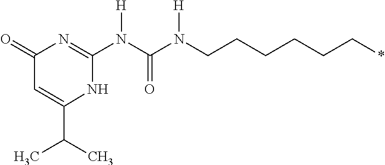

R1=4,4′-methylenebiscyclohexylene, R2=methyl and R4=H, which results in the unit of formula:

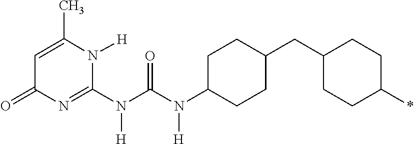

Particularly preferably, in formula (Ib), R1 represents the radical -isophorone-, R2=methyl and R3=—(CH₂)₂OCO—NH-isophorone-, which results in the divalent unit of formula:

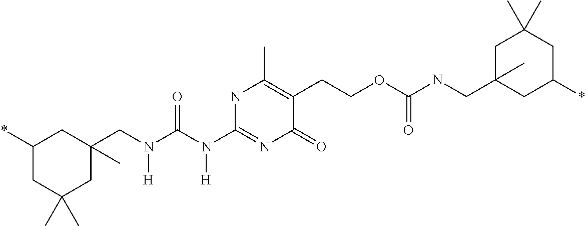

The joining groups bearing a single isocyanate function may be of formula:

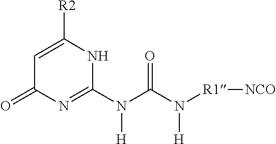

in which R1 and R2 are as defined above; and in particular:
R1 represents -isophorone-, —(CH₂)₆—, —CH₂CH(CH₃)—CH₂—C(CH₃)₂—CH₂—CH₂, 4,4′-methylenebiscyclohexylene or 2-methyl-1,3-phenylene; and/or
R2 represents H, CH₃, ethyl, C₁₃H₂₇, C₇H₁₅, phenyl, isopropyl, isobutyl, n-butyl, tert-butyl, n-propyl, or else —CH(C₂H₅)(C₄H₉).

Preferably, the joining groups may be chosen from the following groups:

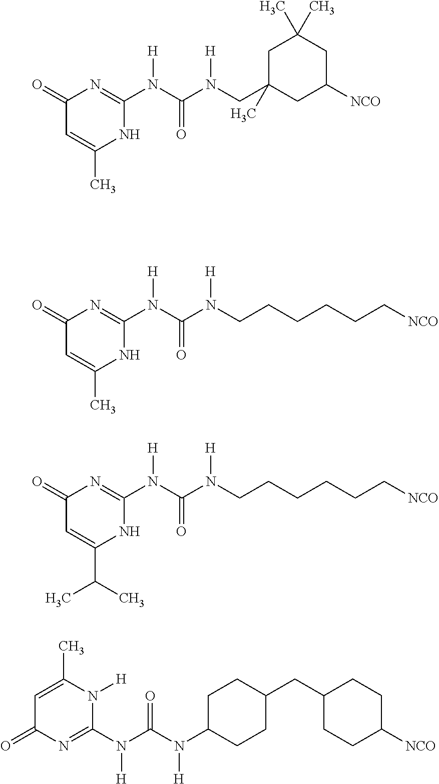

The joining groups bearing two isocyanate functions may be of formula:

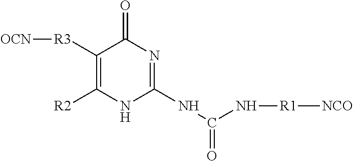

in which R1, R2 and R3 are as defined above, and in particular:
R1 represents -isophorone-, —(CH₂)₂—, —(CH₂)₆—, —CH₂CH(CH₃)—CH₂—C(CH₃)₂—CH₂—CH₂, 4,4′-methylenebiscyclohexylene or 2-methyl-1,3-phenylene; and/or
R2 represents H, CH₃, ethyl, C₁₃H₂₇, C₇H₁₅, phenyl, isopropyl, isobutyl, n-butyl, tert-butyl, n-propyl, or else —CH(C₂H₅)(C₄H₉); and/or
R3 represents a divalent radical —R′3-O—C(O)—NH—R′4- in which R′3 and R′4, which may be identical or different, represent a divalent carbon-based radical chosen from a linear or branched C₁-C₃₀ alkyl group or a C₄-C₁₂ cycloalkyl group or a C₄-C₁₂ aryl group; or mixtures thereof; and in particular R′3 represents a C₁-C₄ alkylene, in particular 1,2-ethylene, and R′4 represents the divalent radical derived from isophorone.

A joining group which is most particularly preferred is that of formula:

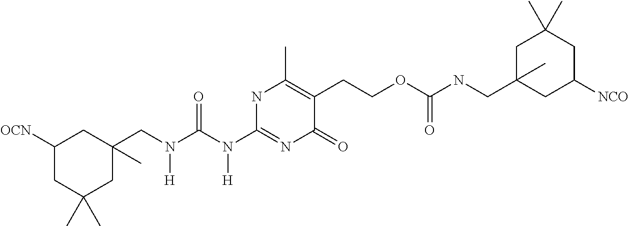

Among the joining groups bearing an imidazole group, mention may be made of the following compound:

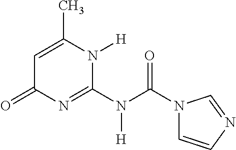 [SMILES: Cc1cc(=O)nc(NC(=O)n2ccnc2)[nH]1]

According to one particular embodiment of the invention, the joining groups may be attached to the oil via functionalization of the joining group with an isocyanate or imidazole.

According to another embodiment, it is possible to carry out the reverse reaction by prefunctionalizing the oil with a diisocyanate.

As mentioned above (1^{st} embodiment), the supramolecular compound A according to the invention (which may, for convenience, also be referred to as supramolecular oil in the present text) can thus result from the chemical reaction between an oil (HB)—(OH)ₘ(NH₂)ₙ and a joining group (G)-(NCO)ₚ or (G)-(imidazole)ₚ.

Preferably, the oil comprises only hydroxyl functions and the joining group comprises 1 or 2 isocyanate functions, which results in the following reactions:

(HB)—(OH)ₘ+OCN-(G)-NCO→(HB)—OC(O)NH-(G)-NHC(O)—(HB)

(HB)—(OH)ᵣ+(G)-NCO→(HB)—OC(O)NH-(G)

with m=integer greater than or equal to 1.

Preferably, the degree of grafting of the free OHs of the oil is between 1% and 100%, in particular between 20% and 99%, and better still between 50% and 95%; preferably, this degree is 100% (all the free OHs are functionalized with a joining group), in particular when the oil initially comprises only one OH function.

The supramolecular compound A according to the invention can be prepared by means of the processes normally used by those skilled in the art to form a urethane bond, between the free OH functions of the oil and the isocyanate functions borne by the joining group. By way of illustration, a general preparation process consists in:
making sure that the oil to be functionalized does not comprise residual water;
optionally heating the oil comprising at least one reactive function, in particular OH, to a temperature which can be between 30° C. and 140° C.;
adding the joining group bearing the reactive functions, in particular isocyanate functions;
optionally stirring the mixture, under a controlled atmosphere, at a temperature of the order of 100-130° C.; for 1 to 24 hours;
monitoring, by infrared spectroscopy, the disappearance of the band characteristic of the isocyanates (between 2500 and 2800 cm⁻¹), so as to halt the reaction at the complete disappearance of the peak, and then allowing the final product to return to ambient temperature.

The reaction can be carried out in the presence of a solvent or of a mixture of solvents, in particular methyltetrahydrofuran, tetrahydrofuran, toluene or butyl acetate, or even propylene carbonate, in particular as cosolvent; the reaction can also be carried out without solvent, it being possible for the oil to then act as a solvent.

It is also possible to add a catalyst conventional for the formation of a urethane bond. By way of example, mention may be made of dibutyltin dilaurate.

The supramolecular compound A may, at the end, be washed and dried, or even purified, according to the general knowledge of those skilled in the art.

According to the 2^{nd} embodiment, the reaction can comprise the following steps:

(i) functionalization of the oil with a diisocyanate according to the reaction scheme:

(HB)—OH(1 eq.)+NCO—X—NCO(1 eq.)→(HB)—OC(O)—NH—X—NCO

then
(iia) either reaction with 6-methylisocytosine:

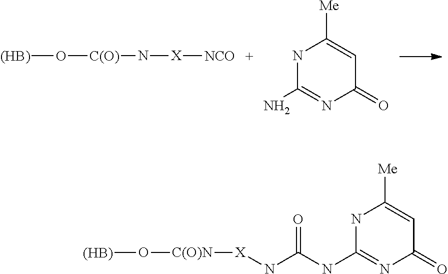

or
(iib) either reaction with 5-hydroxyethyl-6-methylisocytosine:

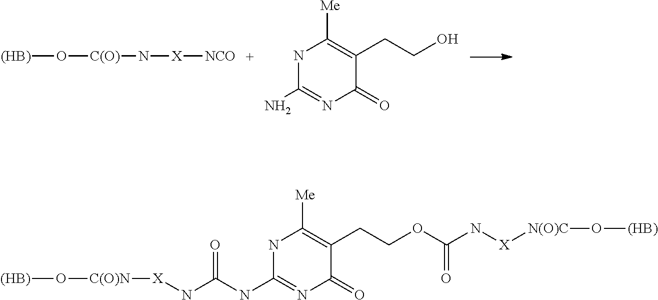

An illustration of such a reaction is given in Folmer et al., Adv. Mater, 12, 874-78 (2000).

The supramolecular compounds A according to the invention (also known as supramolecular oils) can in particular correspond to the following structures:
ureidopyrimidone-functionalized octyldodecanol of structure:

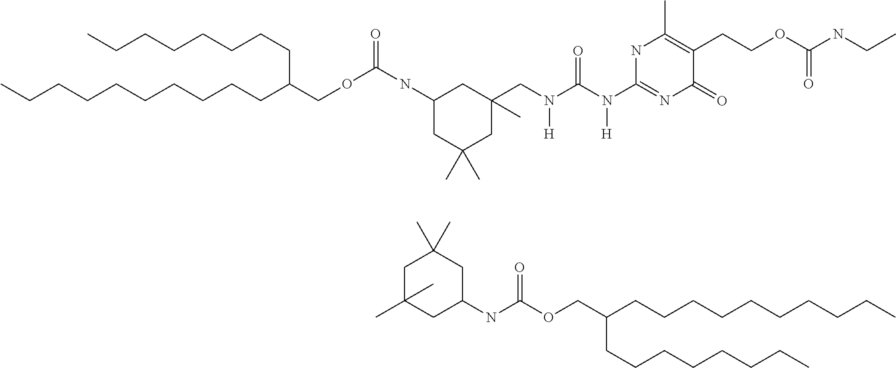

or else of structure:

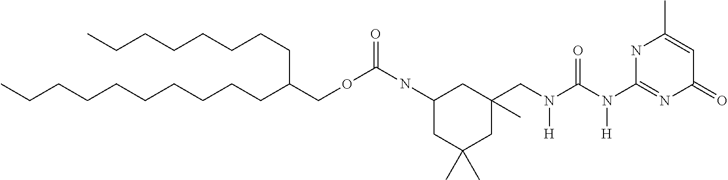 [SMILES: CCCCCCCCCCC(CCCCCCCC)COC(=O)NC1CC(C)(C)CC(C)(CNC(=O)Nc2nc(=O)cc(C)[nH]2)C1]

ureidopyrimidone-functionalized diisostearyl malate of structure:

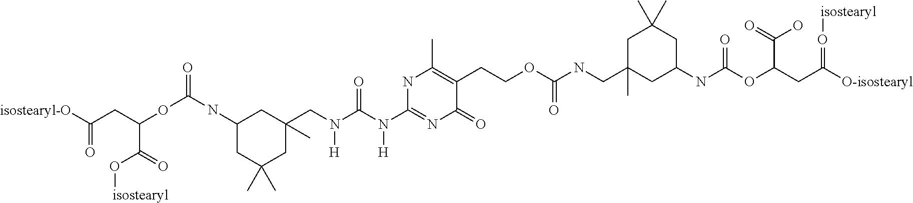

or else of structure:

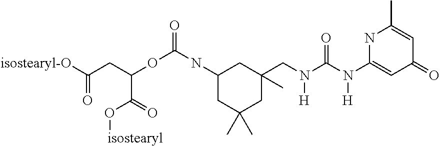

ureidopyrimidone-functionalized castor oil of structure:

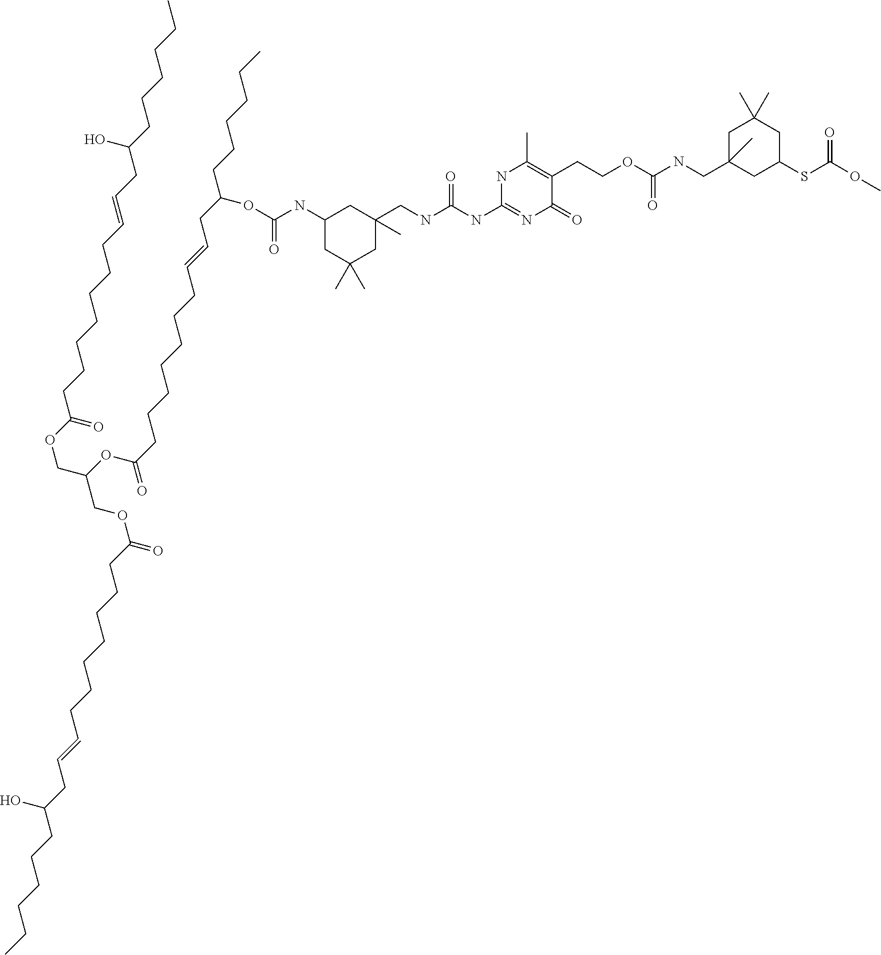

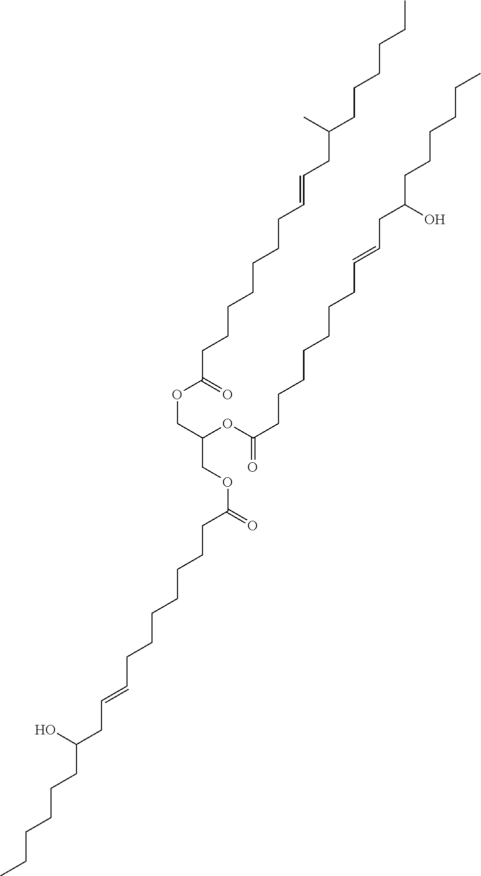 [SMILES: CCCCCCC(C)C/C=C/CCCCCCCC(=O)OCC(COC(=O)CCCCCCC/C=C/CC(O)CCCCCC)OC(=O)CCCCCCC/C=C/CC(O)CCCCCC]

or else of structure:

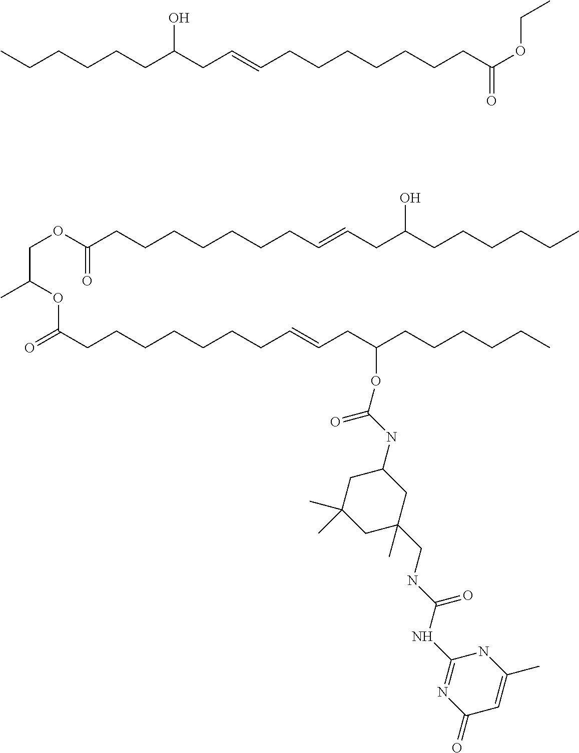

ureidopyrimidone-functionalized 2-hexyldecanol of structure:

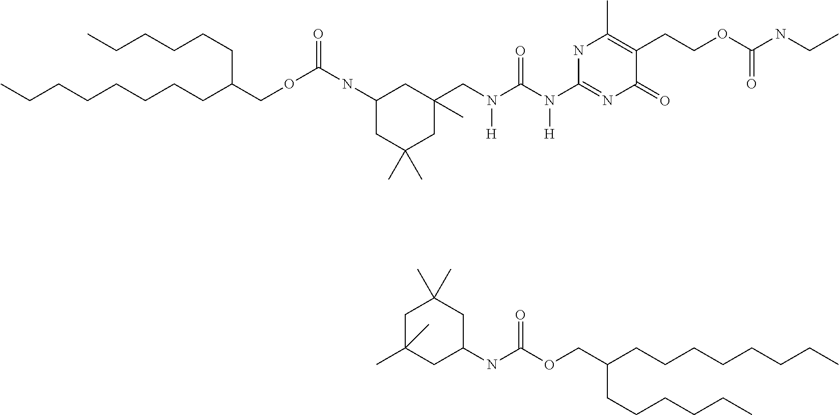

or else of structure:

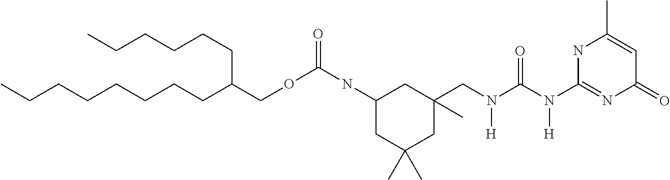 [SMILES: CCCCCCCCC(CCCCCC)COC(=O)NC1CC(C)(C)CC(C)(CNC(=O)Nc2nc(=O)cc(C)[nH]2)C1]

ureidopyrimidone-functionalized 2-decyltetradecanol of structure:

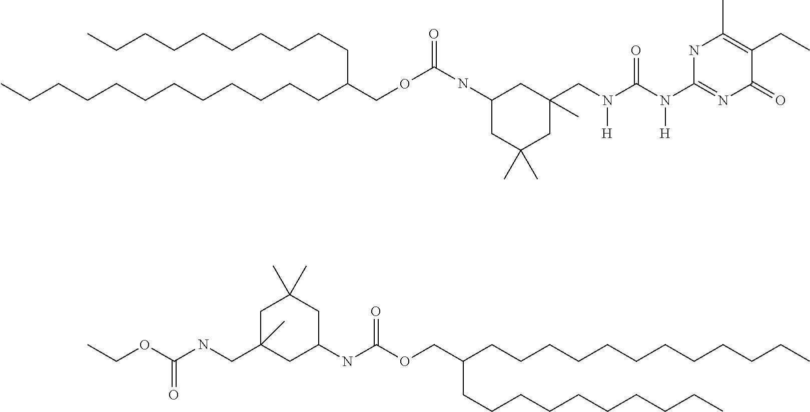

or else of structure:

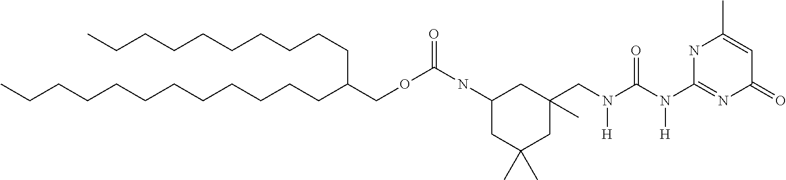 [SMILES: CCCCCCCCCCCCC(CCCCCCCCCC)COC(=O)NC1CC(C)(C)CC(C)(CNC(=O)Nc2nc(=O)cc(C)[nH]2)C1]

ureidopyrimidone-functionalized lauryl alcohol of structure:

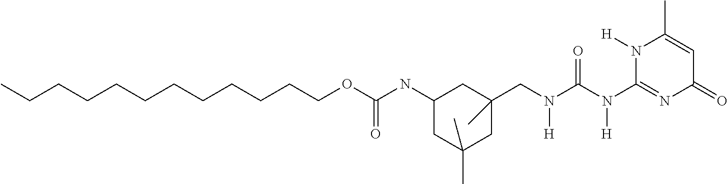

ureidopyrimidone-functionalized cetyl alcohol of structure:

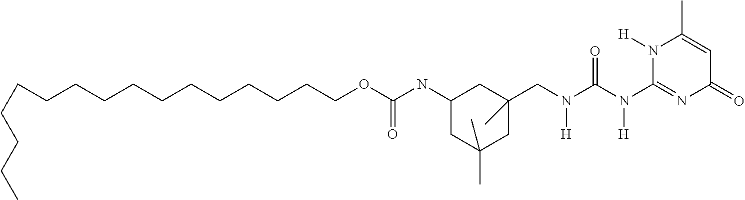

Preferably, compound A according to the invention has a viscosity, measured at 125° C., of between 30 and 6000 mPa·s, in particular between 150 and 4000 mPa·s, or even between 500 and 3500 mPa·s and even better still between 750 and 3000 mPa·s.

The number-average molecular weight (Mn) of the compound according to the invention is preferably between 180 and 8000, preferably 200 to 6000, or even from 300 to 4000, and even better still from 400 to 3000, preferentially from 500 to 1500.

The supramolecular compound A according to the invention (also referred to as “supramolecular compound derived from an oil” or “H-bond-generating oil” or else supramolecular oil) is advantageously soluble in the cosmetic oily media normally used, and in particular in plant oils, C₆-C₃₂ alkanes, C₈-C₃₂ fatty esters, C₂-C₇ short esters, C₈-C₃₂ fatty alcohols, and more particularly in media comprising at least isododecane, parleam, isononyl isononanoate, octyldodecanol, C₁₂-C₁₅ alkyl benzoate, butyl acetate, ethyl acetate, alone or as a mixture.

The term “soluble” is intended to mean that the supramolecular compound A forms a clear solution in at least one solvent chosen from isododecane, parleam, isononyl isononanoate, octyldodecanol, C₁₂-C₁₅ alkyl benzoate, butyl acetate and ethyl acetate, in a proportion of at least 50% by weight, at 25° C.

Said supramolecular compound A (also referred to as supramolecular oil), alone or as a mixture, may be present in the compositions according to the invention in an amount which may be between 0.5% and 99% by weight, preferably between 0.5% and 50% by weight, in particular between 1% and 40% by weight, or even between 1.5% and 20% by weight, and better still between 2% and 15% by weight, relative to the weight of the final cosmetic composition.

Advantageously, in particular in the case of makeup or care compositions, such as makeup compositions for the lips or the skin, a composition according to the invention may comprise from 0.1% to 60% by weight of supramolecular compound A (also referred to as supramolecular oil), relative to the total weight of the composition.

In particular, it may comprise from 0.2% to 50% by weight of supramoleclar compound A, relative to the total weight of the composition.

More particularly, it may comprise from 0.5% to 40% by weight of supramolecular compound A, relative to the total weight of the composition.

### Supramolecular Polymer (Compound B)

The cosmetic compositions according to the invention thus comprise a polyalkene-based (i.e. polyolefin-based) supramolecular polymer, also referred to as “compound B”.

For the purpose of the present invention, the expression “polyalkene-based supramolecular polymer” is intended to mean a polymer resulting from the reaction, in particular from the condensation, of at least one polyalkene polymer functionalized with at least one reactive group, with at least one joining group functionalized with at least one reactive group capable of reacting with the reactive group(s) of the functionalized polyalkene polymer, said joining group being capable of forming at least 3 H (hydrogen) bonds, preferably at least 4 H bonds, preferentially 4 H bonds.

Preferably, said functionalized polyalkene is hydrogenated.

The term “polyalkene” or “polyolefin” is intended to mean a polymer resulting from the polymerization of at least one monomer of alkene type, comprising an ethylenic unsaturation, it being possible for said monomer to be pendant or in the main chain of said polymer. The term “polyalkene” or “polyolefin” thus covers polymers which may or may not comprise a double bond. Preferably, the supramolecular polymers used according to the invention are prepared from a polymer resulting from the polymerization of an alkene comprising at least two ethylenic unsaturations.

The supramolecular polymer according to the invention is capable of forming a supramolecular polymeric chain or network by (self) assembly of said polymer according to the invention with at least one other polymer according to the invention, which may be identical or different, each assembly involving at least one pair of identical or different paired joining groups, borne by each of the polymers according to the invention.

For the purpose of the invention, the term “joining group” is intended to mean any group comprising groups which donate or accept H bonds, and capable of establishing at least three H bonds, preferably at least 4 H bonds, preferably 4 H bonds, with a partner joining group, which may be identical or different. These joining groups may be lateral to the polymer backbone (in a side branch), and/or borne by the ends of the polymer backbone, and/or in the chain forming the polymer backbone. They may be distributed in a random or controlled manner.

### Functionalized Polyalkene

The polyalkene polymers are functionalized with at least one reactive group, preferably with at least two reactive groups. The functionalization is preferably carried out at the chain ends. The term telechelic polymers is then used.

The functionalization groups, or reactive groups, may be attached to the polyalkene polymer via linkers, preferably linear or branched C₁-C₄ alkylene groups, or directly via a single bond.

Preferably, the functionalized polyalkene polymers have a number-average molecular weight (Mn) of between 1000 and 8000.

More preferably, they have a number-average molecular weight of between 1000 and 5000, or even between 1500 and 4500.

More preferably, they have a number-average molecular weight of between 2000 and 4000.

In particular, the functionalized polyalkene polymer capable of forming all or part of the polymer backbone of the supramolecular polymer according to the invention (it preferably forms the entire backbone of the polymer) is of formula HX—P—X′H in which:
XH and X′H are reactive functions, with X and X′, which may be identical or different, chosen from O, S, NH, NCO or NRₐ, Rₐ representing a C₁-C₆ alkyl group; preferably, X and/or X′ denote O; preferentially, X and X′ denote O;
P represents a homopolymer or copolymer which can be obtained by polymerisation of one or more linear, cyclic and/or branched, monounsaturated or polyunsaturated, C₂-C₁₀, preferably C₂-C₄, alkenes; P preferably represents a homopolymer or copolymer which can be obtained by polymerisation of one or more monounsaturated, linear or branched C₂-C₄ alkenes.

Preferably, the functionalized polyalkene polymer, capable of forming all or part of the polymer backbone of the supramolecular polymer according to the invention (it preferably forms the entire backbone of the polymer), is of formula HO—P—OH in which:
P represents a homopolymer or a copolymer which can be obtained by polymerisation of one or more linear, cyclic and/or branched, polyunsaturated (preferably diunsaturated), C₂-C₁₀, preferably C₂-C₄, alkenes.

P preferably represents a homopolymer or a copolymer which can be obtained by polymerisation of one or more diunsaturated, linear or branched, C₂-C₄ alkenes.

More preferably, P represents a polymer chosen from a polyethylene, a polybutylene, a polybutadiene (such as a 1,4-polybutadiene or a 1,2-polybutadiene), a polyisoprene, a poly(1,3-pentadiene), a polyisobutylene, and copolymers thereof, and in particular a poly(ethylene/butylene).

According to one preferred embodiment, P represents a poly(ethylene/butylene) copolymer.

The preferred poly(ethylene/butylene)s are copolymers of 1-butene and of ethylene. They can be represented schematically by the sequence of following units: [—CH₂—CH₂—] and [—CH₂CH(CH₂—CH₃)—].

According to a second preferred embodiment, P is a polybutadiene homopolymer, preferably chosen from a 1,4-polybutadiene or a 1,2-polybutadiene.

The polybutadienes may be 1,4-polybutadienes or 1,2-polybutadienes, which can respectively be represented schematically by the sequences of following units: [—CH₂—CH═CH—CH₂—](1,4-polybutadienes) and [—CH₂—CH(CH═CH₂)—](1,2-polybutadienes). Preferably, they are 1,2-polybutadienes. Preferably, P is a 1,2-polybutadiene homopolymer.

According to another embodiment, P is a polyisoprene. The polyisoprenes can be represented schematically by the sequences of following units:

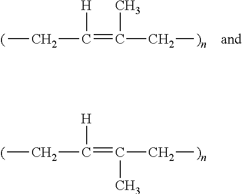

It is very obviously possible to also use a mixture of above units, in order to form copolymers.

The functionalized polyalkene polymers may be hydrogenated, in particular completely hydrogenated, in order to avoid risks of crosslinking. Preferably, the functionalized polyalkene polymers used in the compositions according to the invention are hydrogenated.

The supramolecular polymers may also comprise in their structure other units derived from other monomers. As comonomers, mention may in particular be made of styrene or monomers having an epoxy group. In one preferred embodiment, they do not comprise them and therefore consist solely of polyalkene polymers (100%) to form the polymer backbone. In particular, they are prepared only from polyalkenes P as defined above, with P preferably representing a homopolymer or a copolymer which can be obtained by polymerisation of one or more monounsaturated, linear or branched, C₂-C₄ alkenes, P more preferably representing a polymer chosen from a polyethylene, a polybutylene, a polybutadiene, a polyisoprene, a poly(1,3-pentadiene), a polyisobutylene, and copolymers thereof, and preferentially a poly(ethylene/butylene).

The polyalkene polymers are functionalized with at least one reactive group, preferably with at least two reactive groups. The functionalization is preferably carried out at the end of chains. The term telechelic polymers is then used. The reactive group may be attached to the polyalkene polymer via linkers, preferably linear or branched C₁-C₄ alkylene groups, or directly via a single bond. As reactive group, mention may be made of OH, NH₂, NHR, SH or NCO functions. Preferably, the polyalkene polymers can be functionalized with OH groups, preferably at the ends.

Preferably, they exhibit a functionality with respect to hydroxyl ends of 1.8 to 3, and preferably in the region of 2.

Preferably, the polyalkene polymers are hydrogenated and functionalized with at least two reactive groups X and X′ as defined above, with X and/or X′ denoting O. Preferably, X and X′ denote O.

Among the functionalized polyalkene polymers which are preferred, mention may be made of polydienes, preferably hydrogenated polydienes, comprising hydroxyl functions, preferably comprising hydroxyl ends, and polyolefins comprising hydroxyl ends, and in particular chosen from homopolymers and copolymers of polybutadiene, polyisoprene and of poly(1,3-pentadiene).

The polydienes comprising hydroxyl ends and in particular defined, for example, in FR2782723. They may be chosen from homopolymers and copolymers of polybutadiene, polyisoprene and of poly(1,3-pentadiene). Mention will in particular be made of the hydroxylated polybutadienes sold by the company Sartomer, such as Krasol® Resins and Poly Bd® Resins.

Preferably, they are dihydroxylated hydrogenated 1,2-polybutadiene homopolymers, such as the Nisso-PB I, GI3000, GI2000 and GI1000 range sold by the company Nisso, which can be represented schematically by the following formula:

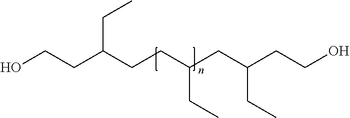

Preferably, “n” is between 14 and 105, preferably between 20 and 85. These polymers have the following number-average molecular weights: GI3000 of Mn=4700, GI2000 of Mn=3300 and GI1000 of Mn=1500. These values were measured by GPC according to the following protocol:

### Protocol for Determining the Molecular Weights of the Supramolecular Polymer by GPC

Determination of the number-average Mn and weight-average Mw molecular weights and also of the Mw/Mn polydispersity index in polystyrene equivalent.

### Preparation of the Calibration Solutions

The polystyrene standards are prepared using the Varian kits (ref: PS—H (PL2010-0200))

### The Weights of the Standards are the Following:

### PS 6035000-PS 3053000-PS 915000-PS 483000-PS 184900-PS 60450-PS 19720-PS 8450-PS 3370-PS 1260-PS 580

100 μl of each of the solutions are injected into the column to be calibrated.

### Preparation of the Sample:

A solution at 0.5% of dry matter is prepared in THF.

The solution is prepared approximately 24 h before injection.

The solution is filtered through a Millex FH filter (0.45 μm).

The solution is injected into the column.

### Chromatographic Conditions:

Columns: PL Rapid M (batch 5M-Poly-008-15) from Polymer Labs
PL-gel HTS-D (batch 5M-MD-72-2) from Polymer Labs
PL-gel HTS-F (10M-2-169B-25) from Polymer Labs
PL-Rapid-F (6M-0L1-011-6) from Polymer Labs
   Length: 150 mm—internal diameter: 7.5 mm
   Pump: Waters isocratic M1515

### Eluent: THF

Flow rate: 1 ml/min
Temperature: ambient
Injection: 100 μl at 0.5% of AM in the eluent
Detection: RI 64 mV (Waters 2424 refractometer)
Temperature: 45° C.
UV at 254 nm in 0.1 OD (Waters 2487 UV detector)
   Integrator: Empower option GPC

### Determination of Molecular Weights

The average molecular weights are determined by plotting the calibration curve: log molecular weight=f (elution volume at the top of the RI detection peak) and using the Empower option GPC software from Waters.

Among the polyolefins comprising hydroxyl ends, mention may preferentially be made of polyolefins, which are homopolymers or copolymers, comprising α,ω-hydroxyl ends, such as polyisobutylenes comprising α,ω-hydroxyl ends; and copolymers of formula:

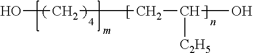

in particular those sold by Mitsubishi under the Polytail trade mark.

### Joining Group

The supramolecular polymers according to the invention also have, in their structure, at least one residue of a joining group capable of forming at least 3 H bonds, preferably at least 4 H bonds, said joining group being initially functionalized with at least one reactive group.

Unless otherwise specified, the term “joining group” is intended to mean, in the present description, the group without its reactive function.

The reactive groups are attached to the joining group via linkers L or directly via a single bond.

Preferably, the linker (L) is a saturated or unsaturated, C₁-C₂₀ divalent carbon-based group, in particular chosen from a linear or branched C₁-C₂₀ alkylene, a C₅-C₂₀ (alkyl)cycloalkylene alkylene (preferably cyclohexylene methylene), a C₁₁-C₂₀ alkylene-biscycloalkylene (preferably alkylene-biscyclohexylene), a C₆-C₂₀ (alkyl)arylene, and an alkylene-bisarylene (preferably an alkylene-bisphenylene), it being possible for the linker L to be substituted with at least one alkyl group and/or to optionally comprise 1 to 4 heteroatoms N and/or O, in particular in the form of an NO₂ substituent.

Preferably, the linker is chosen from a phenylene; 1,4-nitrophenyl; 1,2-ethylene; 1,6-hexylene, 1,4-butylene; 1,6-(2,4,4-trimethylhexylene); 1,4-(4-methylpentylene); 1,5-(5-methylhexylene); 1,6-(6-methylheptylene); 1,5-(2,2,5-trimethylhexylene); 1,7-(3,7-dimethyloctylene); -isophorone-; 4,4′-methylenebiscyclohexylene; tolylene; 2-methyl-1,3-phenylene; 4-methyl-1,3-phenylene or 4,4-biphenylenemethylene group.

Preferably, the linker is chosen from the groups:
C₅-C₂₀ (alkyl)cycloalkylene alkylene, such as isophorone,
C₁₁-C₂₅ alkylene-biscycloalkylene, such as 4,4′-methylenebiscyclohexylene,
C₁-C₂₀ alkylene, such as —(CH₂)₂—; —(CH₂)₆—; —CH₂CH(CH₃)—CH₂—C(CH₃)₂—CH₂—CH₂, and
C₆-C₂₀ (alkyl)phenylene, such as 2-methyl-1,3-phenylene.

Preferably, L is chosen from: -isophorone-; —(CH₂)₂—; —(CH₂)₆—; —CH₂CH(CH₃)—CH₂—C(CH₃)₂—CH₂—CH₂; 4,4′-methylenebiscyclohexylene and 2-methyl-1,3-phenylene.

According to one particularly preferred embodiment, the linker is an alkylcycloalkylene alkylene.

Preferably, according to this embodiment, the linker is an isophorone group. The term “isophorone” is intended to mean the following group:

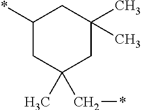 [SMILES: *CC1(C)CC(*)CC(C)(C)C1]

Said reactive groups functionalizing the joining group should be capable of reacting with the reactive group(s), in particular —OH group(s), borne by the functionalized polyalkene.

As reactive groups, mention may be made of isocyanate (—N═C═O) or thioisocyanate (—N═C═S) groups. The reactive group is preferably an —N═C═O (isocyanate) group.

The functionalized joining groups capable of forming at least 3 H bonds can comprise at least 3 identical or different functional groups preferably at least 4, chosen from:

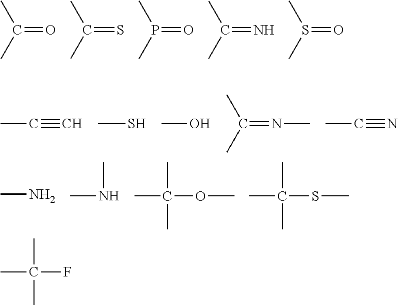

and preferably chosen from

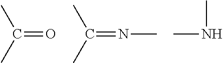

These functional groups can be categorized into two categories:
functional groups which donate H bonds:

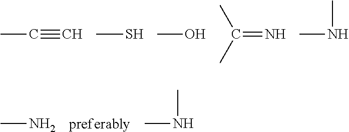

functional groups which accept H bonds:

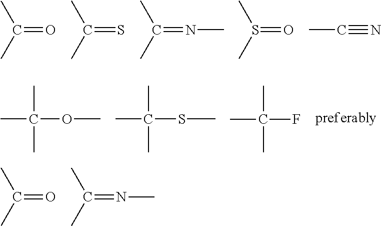

The joining groups capable of forming at least 3 H bonds form a base structural element comprising at least 3 functional groups, preferably at least 4 functional groups, and more preferentially 4 functional groups capable of establishing H bonds. Said base structural elements capable of establishing H bonds can be represented schematically in the following way:

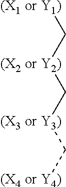

where Xᵢ is a functional group which accepts H bonds (which may be identical or different) and Yᵢ is a functional group which donates H bonds (which may be identical or different).

Thus, each structural element must be able to establish H bonds with one or more partner structural elements which are identical (i.e. self-complementary) or different, such that each pairing of two partner structural elements takes place by formation of at least three H bonds, preferably at least four H bonds, and more preferentially 4 H bonds.

An acceptor of protons X will be paired with a donor of protons Y. Several possibilities are thus offered, for example pairing of:

XXXX with YYYY;
XXXY with YYYX;
XXYX with YYXY;
XYYX with YXXY;
XXYY with YYXX, which is optionally self-complementary;
XYXY with YXYX, which is optionally self-complementary.

Preferably, the joining groups can establish 4 H bonds with an identical (or self-complementary) partner group, among which are 2 donor bonds (for example NH) and 2 acceptor bonds (for example CO and —C═N—).

Preferably, the joining groups capable of forming at least 3, or even at least 4 H bonds are chosen from the following family, it being understood that all the tautomeric forms are included:
ureidopyrimidones of formula

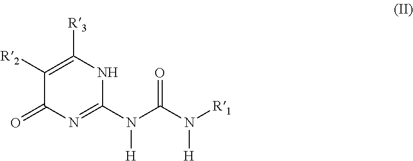

In this formula, the meaning of the radicals is the following:

R′₁ represents a single bond, a hydrogen atom, a halogen atom or a linear, branched and/or cyclic, saturated or unsaturated, optionally aromatic, C₁-C₃₀ monovalent carbon-based (in particular alkyl) group which can contain one or more heteroatoms such as O, S, or N;

The R′₁ radical may in particular be a C₄-C₁₂ cycloalkyl group; a linear or branched C₁-C₃₀ alkyl group or a C₄-C₁₂ aryl group; optionally substituted with an amino, thio and/or hydroxyl function.

Preferably, R′₁ is a C₄H₉; phenyl; 1,4-nitrophenyl; 1,2-ethylene; 1,6-hexylene; 1,4-butylene; 1,6-(2,4,4-trimethylhexylene); 1,4-(4-methylpentylene); 1,5-(5-methylhexylene; 1,6-(6-methylheptylene); 1,5-(2,2,5-trimethylhexylene); 1,7-(3,7-dimethyloctylene); -isophorone-; 4,4′-methylenebiscyclohexylene; tolylene; 2-methyl-1,3-phenylene; 4-methyl-1,3-phenylene or 4,4-bisphenylenemethylene group; or a single bond.

Preferentially, R′₁ represents -isophorone-; —(CH₂)₂—; —(CH₂)₆—; —CH₂CH(CH₃)—CH₂—C(CH₃)₂—CH₂—CH₂; 4,4′-methylenebiscyclohexylene; 2-methyl-1,3-phenylene, or a single bond;

R′₂ represents a single bond, a divalent group of C₁-C₆ alkylene type, or a monovalent group chosen from a hydrogen atom or a linear, branched and/or cyclic, saturated or unsaturated, optionally aromatic, C₁-C₃₀ monovalent hydrocarbon-based group which can contain one or more heteroatoms such as O, S or N;

Preferably, R′₂ can be a single bond or H, a C₁-C₃₀ alkyl group; a C₄-C₁₂ cycloalkyl group; a C₄-C₁₂ aryl group; a (C₄-C₁₂)aryl(C₁-C₁₂)alkyl group; these groups being optionally substituted with an amino, thio and/or hydroxyl function. Preferentially, R′₂ represents H, CH₃, CH₂OH, (CH₂)₂—OH, C₁₃H₂₇, C₇H₁₅ or phenyl; or a single bond;

R′₃ represents a hydrogen atom or a linear, branched and/or cyclic, saturated or unsaturated, optionally aromatic, C₁-C₃₀ monovalent hydrocarbon-based group which can contain one or more heteroatoms such as O, S or N;

Preferably, R′₃ can be a C₄-C₁₂ cycloalkyl group; a linear or branched C₁-C₃₀ alkyl group or a C₄-C₁₂ aryl group; optionally substituted with an amino, thio and/or hydroxyl function; preferentially, R′₃ represents H, CH₃, CH₂OH or (CH₂)₂—OH, and even better still methyl; it being understood that at least one, in particular one or two, of the R′₁ and R′₂ groups is a single bond.

Preferably:
the R′₁ radicals (or else the R′₁ and R′₂ radicals) are single bonds constituting the point of attachment of the joining group to the linker capable of forming at least 3 H bonds (preferably 4) on the rest of the graft. Preferably, said point of attachment is borne only by R′₁ which is a single bond;
the R′₂ radical represents a divalent group chosen from a single bond or a C₁-C₆ alkylene, or a monovalent group chosen from a single bond, a hydrogen atom, or a linear or branched, saturated, C₁-C₁₀ monovalent hydrocarbon-based group which can contain one or more heteroatoms such as O, S, or N, these groups being optionally substituted with a hydroxyl, amino and/or thio function.

Preferably, the R′₂ radical may be a single bond or a monovalent group chosen from H, CH₂OH, (CH₂)₂—OH and CH₃.

According to one particularly preferred embodiment, R′₂ is H;
the R′₃ radical represents a divalent or monovalent group; in particular, R′₃ is chosen from a hydrogen atom or a linear or branched, saturated, C₁-C₁₀ monovalent hydrocarbon-based group which can contain one or more heteroatoms such as O, S or N, said hydrocarbon-based group being optionally substituted with a hydroxyl, amino and/or thio function.

Preferably, the R′₃ radical may be a monovalent group chosen from H, CH₂OH, (CH₂)₂—OH and CH₃.

According to a particularly preferred embodiment, R′₃ is a methyl group.

According to one preferred embodiment, the joining groups are chosen from 2-ureidopyrimidone and 6-methyl-2-ureidopyrimidone.

Preferably, the preferred joining group is 6-methyl-2-ureidopyrimidone.

The joining groups, and in particular the ureidopyrimidone joining groups, can be added directly or else formed in situ during the process for preparing the supramolecular polymer. The first and second preparation modes described hereinafter illustrate, respectively, these two alternatives.

In particular, the functionalized joining groups capable of reacting with the functionalized polyalkene polymer so as to give the supramolecular polymer according to the invention are preferably of formula (III) and preferentially of formula (IV):

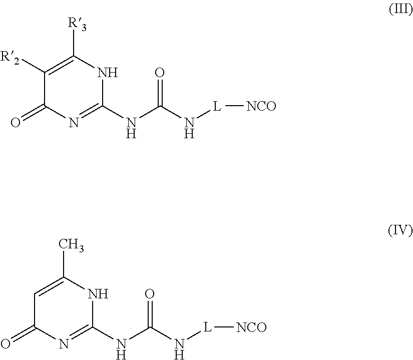

in which:
L is a single bond or a linker as defined above;

In particular, L is a linear, cyclic and/or branched, saturated or unsaturated, or even aromatic, C₁-C₂₀ divalent carbon-based (alkylene) group, optionally comprising 1 to 4 N and/or O heteroatoms, in particular in the form of an NO₂ substituent, and in particular a phenylene; 1,4-nitrophenyl; 1,2-ethylene; 1,6-hexylene; 1,4-butylene; 1,6-(2,4,4-trimethylhexylene); 1,4-(4-methylpentylene); 1,5-(5-methylhexylene); 1,6-(6-methylheptylene); 1,5-(2,2,5-trimethylhexylene); 1,7-(3,7-dimethyloctylene); -isophorone-; 4,4′-methylenebiscyclohexylene; tolylene; 2-methyl-1,3-phenylene; 4-methyl-1,3-phenylene; or 4,4-bisphenylenemethylene group.

Preferably, L is -isophorone-; —(CH₂)₂—; —(CH₂)₆—; —CH₂CH(CH₃)—CH₂—C(CH₃)₂—CH₂—CH₂; 4,4′-methylenebiscyclohexylene or 2-methyl-1,3-phenylene; and better still isophorone;
R′₂ represents a single bond, a divalent group of C₁-C₆ alkylene type, or a monovalent group chosen from a hydrogen atom or a linear, branched and/or cyclic, saturated or unsaturated, optionally aromatic, C₁-C₃₀ monovalent hydrocarbon-based group which can contain one or more heteroatoms such as O, S or N;

Preferably, R′₂ can be a single bond or H, a C₁-C₃₀ alkyl group; a C₄-C₁₂ cycloalkyl group; a C₄-C₁₂ aryl group; a (C₄-C₁₂)aryl(C₁-C₁₂)alkyl group; these groups being optionally substituted with an amino, ester and/or hydroxyl function. Preferentially, R′₂ represents H, CH₃, CH₂OH, (CH₂)₂—OH, C₁₃H₂₇, C₇H₁₅ or phenyl; or a single bond;
R′₃ represents a hydrogen atom or a linear, branched and/or cyclic, saturated or unsaturated, optionally aromatic, C₁-C₃₀ monovalent hydrocarbon-based group which can contain one or more heteroatoms such as O, S or N;

Preferably, R′₃ may be a C₄-C₁₂ cycloalkyl group; a linear or branched C₁-C₃₀ alkyl group or a C₄-C₁₂ aryl group; optionally substituted with an amino, ester and/or hydroxyl function; preferentially, R′₃ represents H, CH₃, CH₂OH or (CH₂)₂—OH; and even better still methyl.

Preferably, the R′₂ radical represents a divalent group chosen from a single bond or a C₁-C₆ alkylene, or a monovalent group chosen from a single bond, a hydrogen atom, or a linear or branched, saturated, C₁-C₁₀ monovalent hydrocarbon-based group which can contain one or more heteroatoms such as O, S or N, these groups being optionally substituted with a hydroxyl, amino and/or thio function.

Preferably, the R′₂ radical may be a single bond or a monovalent group chosen from H, CH₂OH, (CH₂)₂—OH and CH₃.

According to one particularly preferred embodiment, R′₂ is H.

Preferably, the R′₃ radical represents a divalent or monovalent group, in particular R′₃ is chosen from a hydrogen atom or a linear or branched, saturated, C₁-C₁₀ monovalent hydrocarbon-based group which can contain one or more heteroatoms such as O, S or N, said hydrocarbon-based group being optionally substituted with a hydroxyl, amino and/or thio function.

Preferably, the R′₃ radical may be a monovalent group chosen from H, CH₂OH, (CH₂)₂—OH and CH₃.

According to one particularly preferred embodiment, R′₃ is a methyl group.

Preferably, L is chosen from the groups:
C₅-C₂₀ (alkyl)cycloalkylene alkylene, such as isophorone,
C₁₁-C₂₅ alkylene-biscycloalkylene, such as 4,4′-methylenebiscyclohexylene,
C₁-C₂₀ alkylene, such as —(CH₂)₂—; —(CH₂)₆—; —CH₂CH(CH₃)—CH₂—C(CH₃)₂—CH₂—CH₂, and
C₆-C₂₀ (alkyl)phenylene, such as 2-methyl-1,3-phenylene.

Preferably, L is chosen from: -isophorone-; —(CH₂)₆— and 4,4′-methylenebiscyclohexylene.

According to one particularly preferred embodiment, the joining group is of formula:

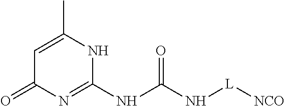

in which L is isophorone.

In one particularly preferred embodiment, the supramolecular polymer of the invention corresponds to the formula:

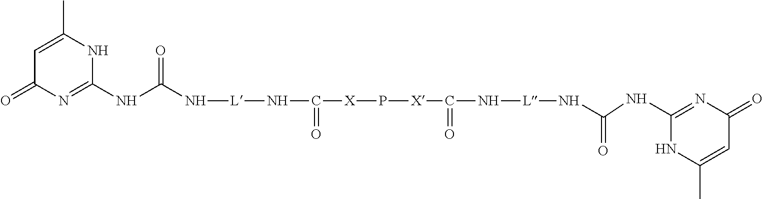

in which:
L′ and L″ have, independently of one another, the meaning indicated above for L;
X, X′═O and P has the meaning indicated above for the functionalized polyalkene polymer.

Preferably, L′ and L″ represent a saturated or unsaturated, linear, cyclic and/or branched, C₁-C₂₀ divalent carbon-based (alkylene) group. Preferably, L′ and L″ are chosen from a linear or branched C₁-C₂₀ alkylene, a C₅-C₂₀ (alkyl)cycloalkylene, an alkylene-biscycloalkylene and a C₆-C₂₀ (alkyl)arylene. Preferably, L′ and L″ represent an -isophorone-; —(CH₂)₂—; —(CH₂)₆—; —CH₂CH(CH₃)—CH₂—C(CH₃)₂—CH₂—CH₂; 4,4′-methylenebiscyclohexylene or 2-methyl-1,3-phenylene group.

Preferably, L′ and L″ are identical.

Preferably, L′ and L″ are an isophorone group.

Preferably, P represents a polyethylene, a polybutylene, a polybutadiene, a polyisoprene, a poly(1,3-pentadiene), a polyisobutylene, or one of their copolymers, in particular a poly(ethylene/butylene), and is preferably hydrogenated.

Preferably, P is a hydrogenated polybutadiene, preferably a hydrogenated 1,2-polybutadiene.

In one particularly preferred embodiment, the supramolecular polymer of the invention corresponds to the formula:

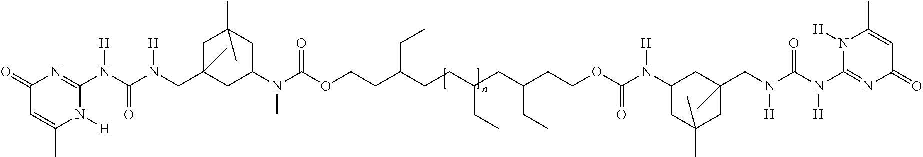

Preferably, n is such that the number-average molecular weight (Mn) of said polymer is between 1000 and 8000, in particular between 1000 and 5000, or even between 1500 and 4500, and even better still between 2000 and 4000.

### Preparation Process

The polymer according to the invention can be prepared by means of the processes usually employed by those skilled in the art, in particular in order to form a urethane bond between the free OH functions of a polyalkene and the isocyanate functions borne by the joining group.

By way of nonlimiting illustration, a first general preparation process consists in:
optionally making sure that the polymer to be functionalized does not comprise residual water;
heating said polymer comprising at least one reactive function, in particular 2 reactive functions, in particular OH, to a temperature which can be between 60° C. and 140° C., it being possible for the hydroxyl number of the polymer to act as reference in order to measure the state of progression of the reaction;
adding, preferably directly, the joining group, in particular the ureidopyrimidone group, bearing the reactive functions, in particular isocyanate reactive functions, such as those described in patent WO 2005/042641; in particular such as the joining groups of cas number 32093-85-9 and 709028-42-2;
optionally stirring the mixture, under a controlled atmosphere, at a temperature of the order of 90-130° C., for 1 to 24 hours;

optionally monitoring, by infrared spectroscopy, the disappearance of the band characteristic of the isocyanates (between 2500 and 2800 cm⁻¹), so as to halt the reaction at the complete disappearance of the peak, and then allowing the final product to return to ambient temperature.

The reaction can also be monitored by quantitative determinations of the hydroxyl functions; it is also possible to add ethanol in order to make sure that the residual isocyanate functions have completely disappeared.

The reaction can be carried out in the presence of a solvent, in particular methyltetrahydrofuran, tetrahydrofuran, toluene, propylene carbonate or butyl acetate. It is also possible to add a catalyst that is conventional for the formation of the urethane bond. By way of example, mention may be made of dibutyltin dilaurate. At the end, the polymer can be washed and dried, or even purified, according to the general knowledge of those skilled in the art.

According to the 2nd method of preparation, which is preferred, the reaction can comprise the following steps:

(i) functionalization of the polymer, preferably predried, with a diisocyanate according to the reaction scheme:

OH-polymer-OH(1 eq.)+NCO—X—NCO(1 eq.)→OCN—X—NH—(O)CO-polymer-OC(O)—NH—X—NCO

The diisocyanate can optionally be in excess with respect to the polymer. This first step can be carried out in the presence of solvent, at a temperature of between 20° C. and 100° C. This first step can be followed by a period of stirring, under a controlled atmosphere, for 1 to 24 hours. The mixture can be optionally heated. The state of progression of this first step can be monitored by quantitative determination of the hydroxyl functions; then

(ii) reaction of the prepolymer obtained above with 6-methylisocytosine of formula:

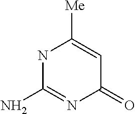

this second step can optionally be carried out in the presence of a cosolvent, such as toluene, butyl acetate or propylene carbonate. The reaction mixture can be heated at between 80° C. and 140° C. for a period of time varying between 1 and 24 hours. The presence of a catalyst, in particular dibutyltin dilaurate, can promote the production of the desired final product.

The reaction can be monitored by infrared spectroscopy, by monitoring the disappearance of the peak characteristic of the isocyanate between 2200 and 2300 cm⁻¹. At the end of the reaction, ethanol can be added to the reaction medium in order to neutralize the possible residual isocyanate functions. The reaction mixture can be optionally filtered. The polymer can also be directly stripped in a cosmetic solvent.

According to one particular embodiment, said supramolecular polymer is solubilized in a hydrocarbon-based, preferably volatile, oil, in particular isododecane.

Thus, the composition of the invention will comprise at least one hydrocarbon-based, preferably volatile, oil, in particular at least isododecane, especially provided by the solution of supramolecular polymer.

In particular, the supramolecular polymer(s) may be present in a composition according to the invention in a content ranging from 0.1% to 99% by weight of dry matter, relative to the total weight of the composition.

According to one preferred variant, the supramolecular polymer(s) may be present in a composition according to the invention in a content ranging from 1% to 80% by weight of dry matter, relative to the total weight of the composition.

According to one preferred variant, the supramolecular polymer(s) may be present in a composition according to the invention in a content ranging from 2% to 70% by weight of dry matter, relative to the total weight of the composition. According to an even more preferred variant, the supramolecular polymer(s) may be present in a composition according to the invention in a content ranging from 3% to 60% by weight of dry matter, relative to the total weight of the composition.

According to an even more preferred variant, the supramolecular polymer(s) may be present in a composition according to the invention in a content ranging from 4% to 50% by weight, relative to the total weight of the composition.

According to an even more preferred variant, the supramolecular polymer(s) may be present in a composition according to the invention in a content ranging from 5% to 40% by weight, relative to the total weight of the composition.

In one particular embodiment of the invention, a makeup composition is provided in the form of a composition for the skin, in particular of the face, or the lips, and the supramolecular polymer(s) may be present therein in a content ranging from 2.5% to 60% by weight of dry matter, relative to the total weight of the composition. According to an even more preferred variant, a makeup composition is provided in the form of a composition for the skin, in particular of the face, or the lips, and the supramolecular polymer(s) may be present therein in a content ranging from 2.5% to 40% by weight of dry matter, relative to the total weight of the composition.

According to an even more preferred variant, a makeup composition is provided in the form of a composition for the skin, in particular of the face, or the lips, and the supramolecular polymer(s) may be present therein in a content ranging from 3% to 30% by weight of dry matter, relative to the total weight of the composition.

Advantageously, a composition according to the invention, in particular in the case of a makeup composition for the skin and/or the lips, comprises at least one supramolecular polymer (compound B) and at least one supramolecular compound A derived from an oil (also referred to as supramolecular oil) in a supramolecular polymer/supramolecular oil weight ratio of between 0.01 and 50.

Preferably, they are present in a weight ratio of between 0.1 and 20.

Even more preferably, they are present in a weight ratio of between 0.5 and 10.

Advantageously, a composition according to the invention, in particular in the case of a makeup composition for the skin and/or the lips, comprises a content of supramolecular polymer (compound B) of between 5% and 99% by weight, relative to the weight of the composition excluding volatile compound(s) (in particular relative to the weight of the composition excluding volatile oil(s), such as isododecane for example).

This content reflects the resulting content of supramolecular polymer(s) in a deposit made with the compositions according to the invention, in particular on keratin materials such as the skin and/or the lips, for example, after evaporation of the volatile compounds.

Preferably, the composition according to the invention, in particular in the case of a makeup composition, comprises a content of supramolecular polymer (compound B) of between 10% and 90% by weight, relative to the weight of the composition excluding volatile compound(s), preferably between 15% and 80%.

The cosmetic compositions according to the invention comprise, moreover, a cosmetically acceptable medium, i.e. a medium compatible with keratin materials such as the skin of the face or of the body, the eyelashes, the eyebrows, the lips and the nails.

### Liquid Fatty Phase

Said medium may comprise a liquid fatty phase, which may comprise at least one compound chosen from volatile or non-volatile carbon-based, hydrocarbon-based and/or silicone and/or fluoro oils and/or solvents of mineral, animal, plant or synthetic origin, alone or as a mixture, provided that they form a uniform, stable mixture and are compatible with the intended use.

According to one preferred embodiment, in particular in the case of the makeup and/or care compositions for keratin materials, in particular such as the lips or the skin, the compositions according to the invention comprise at least one volatile or non-volatile oil.

The term “oil” is intended to mean a water-immiscible, non-aqueous compound which is liquid at ambient temperature (25° C.) and atmospheric pressure (760 mmHg).

For the purpose of the invention, the term “volatile” is intended to mean any compound that is capable of evaporating on contact with keratin materials, or the lips, in less than one hour, at ambient temperature (25° C.) and atmospheric pressure (1 atm). In particular, this volatile compound has a non-zero vapour pressure, at ambient temperature and atmospheric pressure, especially ranging from 0.13 Pa to 000 Pa, in particular ranging from 1.3 Pa to 13 000 Pa, and more particularly ranging from 1.3 Pa to 1300 Pa.

In contrast, the term “non-volatile” is intended to mean a compound that remains on keratin materials or the lips at ambient temperature and atmospheric pressure for at least one hour, and which in particular has a vapour pressure of less than 10⁻³ mmHg (0.13 Pa).

Preferably, the physiologically acceptable medium of the composition according to the invention may comprise, in a liquid fatty phase, at least one oil and/or one solvent which can be chosen, alone or as a mixture, from:

1/esters of monocarboxylic acids with monoalcohols and polyalcohols; advantageously, said ester is a C₁₂-C₁₅ alkyl benzoate or corresponds to the following formula: R′₁—COO—R′₂ in which:

R′₁ represents a linear or branched alkyl radical of 1 to 40 carbon atoms and preferably of 7 to 19 carbon atoms, optionally comprising one or more ethylenic double bonds, optionally substituted, and the hydrocarbon-based chain of which may be interrupted with one or more heteroatoms chosen from N and O and/or one or more carbonyl functions, and
R′₂ represents a linear or branched alkyl radical of 1 to 40 carbon atoms, preferably 3 to 30 carbon atoms and better still 3 to 20 carbon atoms, optionally comprising one or more optionally substituted ethylenic double bonds, and the hydrocarbon-based chain of which may be interrupted with one or more heteroatoms chosen from N and O and/or one or more carbonyl functions.

The term “optionally substituted” means that R′₁ and/or R′₂ may bear one or more substituents chosen, for example, from groups comprising one or more heteroatoms chosen from O and/or N, such as amino, amine, alkoxy and hydroxyl.

Examples of groups R′₁ are those derived from fatty acids, preferably higher fatty acids, chosen from the group formed by acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linolenic acid, linoleic acid, oleostearic acid, arachidonic acid and erucic acid, and mixtures thereof.

Preferably, R′₁ is a branched, unsubstituted alkyl group of 4 to 14 carbon atoms and preferably of 8 to 10 carbon atoms, and R′₂ is a branched, unsubstituted alkyl group of 5 to 15 carbon atoms and preferably of 9 to 11 carbon atoms.

Mention may be made in particular, preferably, of C₈-C₄₈ esters, optionally incorporating in their hydrocarbon-based chain one or more heteroatoms chosen from N and O and/or one or more carbonyl functions; and more particularly purcellin oil (cetostearyl octanoate), isononyl isononanoate, isopropyl myristate, isopropyl palmitate, 2-ethylhexyl palmitate, 2-octyldodecyl stearate, 2-octyldodecyl erucate, isostearyl isostearate, C₁₂ to C₁₅ alkyl benzoate, hexyl laurate or diisopropyl adipate; and heptanoates, octanoates, decanoates or ricinoleates of alcohols or of polyalcohols, for example of fatty alcohols, for instance propylene glycol dioctanoate, and also isopropyl N-lauroyl sarcosinate (especially Eldew-205SL from Ajinomoto); hydroxylated esters, for instance isostearyl lactate or diisostearyl malate; and pentaerythritol esters; branched C₈-C₁₆ esters, especially isohexyl neopentanoate.

2/Hydrocarbon-based plant oils with a high triglyceride content, formed from fatty acid esters of glycerol in which the fatty acids may have varied chain lengths from C₄ to C₂₄, these chains possibly being linear or branched, and saturated or unsaturated; these oils are especially wheatgerm oil, corn oil, sunflower oil, shea oil, castor oil, sweet almond oil, macadamia oil, apricot oil, soyabean oil, rapeseed oil, cottonseed oil, alfalfa oil, poppy seed oil, pumpkin oil, sesame seed oil, marrow oil, avocado oil, hazelnut oil, grapeseed oil, blackcurrant seed oil, evening primrose oil, millet oil, barley oil, quinoa oil, olive oil, rye oil, safflower oil, candlenut oil, passionflower oil, musk rose oil, jojoba oil, palm oil or beauty-leaf oil; or alternatively caprylic/capric acid triglycerides, for instance those sold by the company Stearinerie Dubois or those sold under the names Miglyol 810®, 812® and 818® by the company Dynamit Nobel.

3/Alcohols, and especially C₆-C₃₂ and especially C₁₂-C₂₆ monoalcohols, for instance oleyl alcohol, linoleyl alcohol, linolenyl alcohol, isostearyl alcohol, 2-hexyldecanol, 2-butyloctanol, 2-undecylpentadecanol and octyldodecanol;

4/linear or branched, volatile or non-volatile hydrocarbon-based oils, of synthetic or mineral origin, which may be chosen from hydrocarbon-based oils containing from 5 to 100 carbon atoms, and especially petroleum jelly, polydecenes, hydrogenated polyisobutenes such as parleam, squalane and perhydrosqualene, and mixtures thereof.

Mention may be made more particularly of linear, branched and/or cyclic C₅-C₄₈ alkanes, and preferentially branched C₈-C₁₆ alkanes, for instance C₈-C₁₆ isoalkanes of petroleum origin (also known as isoparaffins); especially decane, heptane, dodecane and cyclohexane; and also isododecane, isodecane and isohexadecane.

5/Volatile or non-volatile silicone oils;

Volatile silicone oils that may be mentioned include linear or cyclic volatile silicone oils, especially those with a viscosity of less than 8 centistokes, and especially containing from 2 to 10 silicon atoms, these silicones optionally comprising alkyl or alkoxy groups containing from 1 to 22 carbon atoms; and in particular octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and methylhexyldimethylsiloxane, and mixtures thereof.

The non-volatile silicone oils that may be used according to the invention may be polydimethylsiloxanes (PDMS), polydimethylsiloxanes comprising alkyl or alkoxy groups, which are pendant and/or at the end of a silicone chain, each group containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

Preferentially, the physiologically acceptable medium of the composition according to the invention comprises, in a liquid fatty phase, at least one oil and/or one solvent chosen, alone or as a mixture, from isododecane, parleam, isononyl isononanoate, octyldodecanol, phenyl trimethicone, C₁₂-C₁₅ alkyl benzoates, butyl and ethyl acetates, and/or D5 (decamethylcyclopentasiloxane).

The liquid fatty phase may also comprise additional oils and/or solvents, which may be chosen, alone or as a mixture, from:
fluoro oils such as perfluoropolyethers, perfluoroalkanes, for instance perfluorodecalin, perfluorodamantanes, perfluoroalkyl phosphate monoesters, diesters and triesters and fluoro ester oils;
oils of animal origin;
C₆ to C₄₀ and especially C₁₀-C₄₀ ethers; propylene glycol ethers that are liquid at ambient temperature, such as propylene glycol monomethyl ether, propylene glycol monomethyl ether acetate or dipropylene glycol mono-n-butyl ether;
C₈-C₃₂ fatty acids, for instance oleic acid, linoleic acid or linolenic acid, and mixtures thereof;
difunctional oils, comprising two functions chosen from ester and/or amide and containing from 6 to 30 carbon atoms, especially 8 to 28 carbon atoms and better still from 10 to 24 carbon atoms, and 4 heteroatoms chosen from O and N; preferably, the amide and ester functions being in the chain;
ketones that are liquid at ambient temperature (25° C.) such as methyl ethyl ketone, methyl isobutyl ketone, diisobutyl ketone, isophorone, cyclohexanone and acetone;
aldehydes that are liquid at ambient temperature, such as benzaldehyde and α-etaldehyde.

The liquid fatty phase may represent 1% to 90% by weight of the composition, especially from 5% to 75% by weight, in particular from 10% to 60% by weight or even from 25% to 55% by weight relative to the total weight of the composition.

### Silicone Compound

According to one preferred embodiment, especially in the case of makeup compositions, in particular for the skin and/or the lips, the composition according to the invention comprises at least one silicone compound having a viscosity of less than 10,000,000 cSt at 25° C. Such a compound is advantageously chosen from silicone gums, volatile silicone oils and non-volatile silicone oils.

According to one particular embodiment, the care and/or makeup composition for the skin and/or the lips comprises at least one silicone compound.

The presence of such a compound in the compositions according to the invention makes it possible to obtain compositions of which the deposit on keratin materials, and in particular on the skin and/or the lips, is not very tacky or not tacky at all.

Its presence also makes it possible to improve the properties of transfer resistance of the deposits, and/or of resistance to attacks, in particular to rubbing. The colour fastness of the deposits can also be improved (resistance to rubbing), as can the comfort and the cosmeticity of the deposit formed (softness, glidance to the touch of the deposit formed).

In particular, the silicone compound under consideration according to the invention may be a silicone oil having a viscosity of between 3 centistokes (cSt) (3×10⁻⁶ m²/s) and 800 000 centistokes (cSt) (800 000×10⁻⁶ m²/s).

Preferably, the silicone compound under consideration according to the invention may be a non-volatile silicone oil having a viscosity of between 9 centistokes (cSt) (3×10⁻⁶ m²/s) and 600 000 centistokes (cSt) (600 000×10⁻⁶ m²/s).

### Silicone Oils

For the purpose of the present invention, the term “silicone oil” is intended to mean an oil comprising at least one silicon atom, and in particular at least one Si—O group.

In particular, the volatile or non-volatile silicone oils that can be used in the invention preferably have a viscosity at 25° C. of less than 800 000 cSt, preferably less than or equal to 600 000 cSt, preferably less than or equal to 500 000 cSt. The viscosity of these silicone oils can be measured according to standard ASTM D-445.

Of course, a composition according to the invention or under consideration according to a process of the invention can contain a mixture of silicone oils only partly made up of such an oil.

The silicone oils that can be used in the compositions of the invention may be volatile and/or non-volatile.

### Volatile Silicone Oils

According to a first embodiment, the compositions according to the invention comprise at least one volatile silicone oil.

The volatile silicone oil that can be used in the invention can be chosen from silicone oils having in particular a viscosity ≦8 centistokes (cSt) (8×10⁻⁶ m²/s).

In addition, the volatile silicone oil that can be used in the invention may be chosen preferably from silicone oils having a flash point ranging from 40° C. to 102° C., preferably having a flash point of greater than 55° C. and less than or equal to 95° C., and preferentially ranging from 65° C. to 95° C.

By way of volatile silicone oils, mention may be made of:
linear or cyclic volatile silicone oils, in particular those having a viscosity ≦8 centistokes (cSt) (8×10⁻⁶ m²/s at 25° C.), and especially having from 2 to 10 silicon atoms, and in particular from 2 to 7 silicon atoms, these silicones optionally comprising alkyl or alkoxy groups having from 1 to 10 carbon atoms.

More particularly, the volatile silicone oils are noncyclic and are in particular chosen from:
noncyclic linear silicones of formula (I): R₃SiO—(R₂SiO)ₙ—SiR₃ in which R, which may be identical or different, denotes:
a saturated or unsaturated hydrocarbon-based radical having from 1 to 10 carbon atoms, preferably from 1 to 6 carbon atoms, optionally substituted with one or more fluorine atoms or with one or more hydroxyl groups, or
a hydroxyl group, it being possible for one of the R radicals to be a phenyl group, n being an integer ranging from 0 to 8, preferably ranging from 2 to 6, and better still ranging from 3 to 5, the silicone compound of formula (I) containing at most 15 carbon atoms;
branched silicones of formula (II) or (III) below:

R₃SiO—[(R₃SiO)RSiO]—(R₂SiO)ₓ—SiR₃ (II)

[R₃SiO]4Si (III)

in which R, which may be identical or different, denotes:
a saturated or unsaturated hydrocarbon-based radical having from 1 to 10 carbon atoms, optionally substituted with one or more fluorine atoms or with one or more hydroxyl groups, or
a hydroxyl group, it being possible for one of the R radicals to be a phenyl group, x being an integer ranging from 0 to 8, the silicone compound of formula (II) or (III) containing at most 15 carbon atoms.

Preferably, for the compounds of formulae (I), (II) and (III), the ratio between the number of carbon atoms and the number of silicon atoms is between 2.25 and 4.33.

The silicones of formulae (I) to (III) can be prepared according to known processes for synthesizing silicone compounds.

Among the silicones of formula (I), mention may be made of:
the following disiloxanes: hexamethyldisiloxane (surface tension=15.9 mN/m), in particular sold under the name DC 200 Fluid 0.65 cst by the company Dow Corning; 1,3-di-tert-butyl-1,1,3,3-tetramethyldisiloxane; 1,3-dipropyl-1,1,3,3-tetramethyldisiloxane; heptylpentamethyldisiloxane, 1,1,1-triethyl-3,3,3-trimethyldisiloxane; hexaethyldisiloxane; 1,1,3,3-tetramethyl-1,3-bis(2-methylpropyl)disiloxane; pentamethyloctyldisiloxane; 1,1,1-trimethyl-3,3,3-tris(1-methylethyl)disiloxane; 1-butyl-3-ethyl-1,1,3-trimethyl-3-propyldisiloxane; pentamethylpentyldisiloxane; 1-butyl-1,1,3,3-tetramethyl-3-(1-methylethyl)disiloxane; 1,1,3,3-tetramethyl-1,3-bis(1-methylpropyl)disiloxane; 1,1,3-triethyl-1,3,3-tripropyldisiloxane; (3,3-dimethylbutyl)pentamethyldisiloxane; (3-methylbutyl)pentamethyldisiloxane; (3-methylpentyl)pentamethyldisiloxane; 1,1,1-triethyl-3,3-dimethyl-3-propyldisiloxane; 1-(1,1-dimethylethyl)-1,1,3,3,3-pentamethyldisiloxane; 1,1,1-trimethyl-3,3,3-tripropyldisiloxane; 1,3-dimethyl-1,1,3,3-tetrakis(1-methylethyl)disiloxane; 1,1-dibutyl-1,3,3,3-tetramethyldisiloxane; 1,1,3,3-tetramethyl-1,3-bis(1-methylethyl)disiloxane; 1,1,1,3-tetramethyl-3,3-bis(1-methylethyl)disiloxane, 1,1,1,3-tetramethyl-3,3-dipropyldisiloxane; 1,1,3,3-tetramethyl-1,3-bis(3-methylbutyl)disiloxane; butylpentamethyldisiloxane; pentaethylmethyldisiloxane; 1,1,3,3-tetramethyl-1,3-dipentyldisiloxane; 1,3-dimethyl-1,1,3,3-tetrapropyldisiloxane; 1,1,1,3-tetraethyl-3,3-dimethyldisiloxane; 1,1,1-triethyl-3,3,3-tripropyldisiloxane; 1,3-dibutyl-1,1,3,3-tetramethyldisiloxane and hexylpentamethyldisiloxane;
   the following trisiloxanes: octamethyltrisiloxane (surface tension=17.4 mN/m), in particular sold under the name DC 200 Fluid 1 cst by the company Dow Corning; 3-pentyl-1,1,1,3,5,5,5-heptamethyltrisiloxane; 1-hexyl-1,1,3,3,5,5,5-heptamethyltrisiloxane; 1,1,1,3,3,5,5-heptamethyl-5-octyltrisiloxane; 1,1,1,3,5,5,5-heptamethyl-3-octyltrisiloxane, in particular sold under the name Silsoft 034 by the company OSI; 1,1,1,3,5,5,5-heptamethyl-3-hexyltrisiloxane (surface tension=20.5 mN/m), in particular sold under the name DC2-1731 by the company Dow Corning; 1,1,3,3,5,5-hexamethyl-1,5-dipropyltrisiloxane; 3-(1-ethylbutyl)-1,1,1,3,5,5,5-heptamethyltrisiloxane; 1,1,1,3,5,5,5-heptamethyl-3-(1-methylpentyl)trisiloxane; 1,5-diethyl-1,1,3,3,5,5-hexamethyltrisiloxane; 1,1,1,3,5,5,5-heptamethyl-3-(1-methylpropyl)trisiloxane; 3-(1,1-dimethylethyl)-1,1,1,3,5,5,5-heptamethyltrisiloxane; 1,1,1,5,5,5-hexamethyl-3,3-bis(1-methylethyl)trisiloxane; 1,1,1,3,3,5,5-hexamethyl-1,5-bis(1-methylpropyl)trisiloxane; 1,5-bis(1,1-dimethylethyl)-1,1,3,3,5,5-hexamethyltrisiloxane; 3-(3,3-dimethylbutyl)-1,1,1,3,5,5,5-heptamethyltrisiloxane; 1,1,1,3,5,5,5-heptamethyl-3-(3-methylbutyl)trisiloxane; 1,1,1,3,5,5,5-heptamethyl-3-(3-methylpentyl)trisiloxane; 1,1,1,3,5,5,5-heptamethyl-3-(2-methylpropyl)trisiloxane; 1-butyl-1,1,3,3,5,5,5-heptamethyltrisiloxane; 1,1,1,3,5,5,5-heptamethyl-3-propyltrisiloxane; 3-isohexyl-1,1,1,3,5,5,5-heptamethyltrisiloxane; 1,3,5-triethyl-1,1,3,5,5-pentamethyltrisiloxane; 3-butyl-1,1,1,3,5,5,5-heptamethyltrisiloxane; 3-tert-pentyl-1,1,1,3,5,5,5-heptamethyltrisiloxane; 1,1,1,5,5,5-hexamethyl-3,3-dipropyltrisiloxane; 3,3-diethyl-1,1,1,5,5,5-hexamethyltrisiloxane; 1,5-dibutyl-1,1,3,3,5,5-hexamethyltrisiloxane; 1,1,1,5,5,5-hexaethyl-3,3-dimethyltrisiloxane; 3,3-dibutyl-1,1,1,5,5,5-hexamethyltrisiloxane; 3-ethyl-1,1,1,3,5,5,5-heptamethyltrisiloxane; 3-heptyl-1,1,1,3,5,5,5-heptamethyltrisiloxane and 1-ethyl-1,1,3,3,5,5,5-heptamethyltrisiloxane;
   the following tetrasiloxanes: decamethyltetrasiloxane (surface tension=18 mN/m), in particular sold under the name DC 200 Fluid 1.5 cst by the company Dow Corning; 1,1,3,3,5,5,7,7-octamethyl-1,7-dipropyltetrasiloxane; 1,1,1,3,3,5,7,7,7-nonamethyl-5-(1-methylethyl)tetrasiloxane, 1-butyl-1,1,3,3,5,5,7,7,7-nonamethyltetrasiloxane; 3,5-diethyl-1,1,1,3,5,7,7,7-octamethyltetrasiloxane; 1,3,5,7-tetraethyl-1,1,3,5,7,7-hexamethyltetrasiloxane; 3,3,5,5-tetraethyl-1,1,1,7,7,7-hexamethyltetrasiloxane; 1,1,1,3,3,5,5,7,7-nonamethyl-7-phenyltetrasiloxane; 3,3-diethyl-1,1,1,5,5,7,7,7-octamethyltetrasiloxane; 1,1,1,3,3,5,7,7,7-nonamethyl-5-phenyltetrasiloxane;
the following pentasiloxanes: dodecamethylpentasiloxane (surface tension=18.7 mN/m), in particular sold under the name DC 200 Fluid cst 2 by the company Dow Corning; 1,1,3,3,5,5,7,7,9,9-decamethyl-1,9-dipropylpentasiloxane; 3,3,5,5,7,7-hexaethyl-1,1,1,9,9,9-hexamethylpentasiloxane; 1,1,1,3,3,5,7,7,9,9,9-undecamethyl-5-phenylpentasiloxane; 1-butyl-1,1,3,3,5,5,7,7,9,9,9-undecamethylpentasiloxane; 3,3-diethyl-1,1,1,5,5,7,7,9,9,9-decamethylpentasiloxane; 1,3,5,7,9-pentaethyl-1,1,3,5,7,9,9-heptamethylpentasiloxane; 3,5,7-triethyl-1,1,1,3,5,7,9,9,9-nonamethylpentasiloxane and 1,1,1-triethyl-3,3,5,5,7,7,9,9,9-nonamethylpentasiloxane;
   the following hexasiloxanes: 1-butyl-1,1,3,3,5,5,7,7,9,9,11,11,11-tridecamethylhexasiloxane; 3,5,7,9-tetraethyl-1,1,1,3,5,7,9,11,11,11-decamethylhexasiloxane and tetradecamethylhexasiloxane;
hexadecamethylheptasiloxane;
octadecamethyloctasiloxane;
eicosomethylnonasiloxane.

Among the silicones of formula (II), mention may be made of:
the following tetrasiloxanes: 2-[3,3,3-trimethyl-1,1-bis[(trimethylsilyl)oxy]disiloxanyl]ethyl; 1,1,1,5,5,5-hexamethyl-3-(2-methylpropyl)-3-[(trimethylsilyl)oxy]trisiloxane; 3-(1,1-dimethylethyl)-1,1,1,5,5,5-hexamethyl-3-[(trimethylsilyl)oxy]trisiloxane; 3-butyl-1,1,1,5,5,5-hexamethyl-3-[(trimethylsilyl)oxy]trisiloxane; 1,1,1,5,5,5-hexamethyl-3-propyl-3-[(trimethylsilyl)oxy]trisiloxane; 3-ethyl-1,1,1,5,5,5-hexamethyl-3-[(trimethylsilyl)oxy]trisiloxane; 1,1,1-triethyl-3,5,5,5-tetramethyl-3-(trimethylsiloxy)trisiloxane; 3-methyl-1,1,1,5,5,5-hexamethyl-3-[(trimethylsilyl)oxy]trisiloxane; 3-[(dimethylphenylsilyl)oxy]-1,1,1,3,5,5,5-heptamethyltrisiloxane; 1,1,1,5,5,5-hexamethyl-3-(2-methylpentyl)-3-[(trimethylsilyl)oxy]trisiloxane; 1,1,1,5,5,5-hexamethyl-3-(4-methylpentyl)-3-[(trimethylsilyl)oxy]trisiloxane; 3-hexyl-1,1,1,5,5,5-hexamethyl-3-[(trimethylsilyl)oxy]trisiloxane and 1,1,1,3,5,5,5-heptamethyl-3-[(trimethylsilyl)oxy]trisiloxane;
the following pentasiloxanes: 1,1,1,3,5,5,7,7,7-nonamethyl-3-(trimethylsiloxy)tetrasiloxane and 1,1,1,3,3,7,7,7-octamethyl-5-phenyl-5-[(trimethylsilyl)oxy]tetrasiloxane;
the following heptasiloxanes: 1,1,1,3,5,5,7,7,9,9,11,11,11-tridecamethyl-3-[(trimethylsilyl)oxy]hexasiloxane.

Among the silicones of formula (III), mention may be made of:
1,1,1,5,5,5-hexamethyl-3,3-bis(trimethylsiloxy)trisiloxane.

Use may also be made of other volatile silicone oils chosen from:
the following tetrasiloxanes: 2,2,8,8-tetramethyl-5-[(pentamethyldisiloxanyl)methyl]-3,7-dioxa-2,8-disilanonane; 2,2,5,8,8-pentamethyl-5-[(trimethylsilyl)methoxy]-4,6-dioxa-2,5,8-trisilanonane; 1,3-dimethyl-1,3-bis[(trimethylsilyl)methyl]-1,3-disiloxanediol; 3-ethyl-1,1,1,5,5,5-hexamethyl-3-[3-(trimethylsiloxy)propyl]trisiloxane and 1,1,1,5,5,5-hexamethyl-3-phenyl-3-[(trimethylsilyl)oxy]trisiloxane (Dow 556 Fluid);
the following pentasiloxanes: 2,2,7,7,9,9,11,11,16,16-decamethyl-3,8,10,15-tetraoxa-2,7,9,11,16-pentasilaheptadecane and silicic acid tetrakis[(trimethylsilyl)methyl]ester;
the following hexasiloxanes: 3,5-diethyl-1,1,1,7,7,7-hexamethyl-3,5-bis[(trimethylsilyl)oxy]tetrasiloxane and 1,1,1,3,5,7,7,7-octamethyl-3,5-bis-[(trimethylsilyl)oxy]tetrasiloxane;
the heptasiloxane: 1,1,1,3,7,7,7-heptamethyl-3,5,5-tris[(trimethylsilyl)oxy]tetrasiloxane;
the following octasiloxanes: 1,1,1,3,5,5,9,9,9-nonamethyl-3,7,7-tris[(trimethylsilyl)oxy]pentasiloxane; 1,1,1,3,5,7,9,9,9-nonamethyl-3,5,7-tris[(trimethylsilyl)oxy]pentasiloxane and 1,1,1,7,7,7-hexamethyl-3,3,5,5-tetrakis[(trimethylsilyl)oxy]tetrasiloxane.

By way of the volatile silicone oils, mention may be made more particularly of decamethylcyclopentasiloxane, in particular sold under the name DC-245 by the company Dow Corning, dodecamethylcyclohexasiloxane, in particular sold under the name DC-246 by the company Dow Corning, octamethyltrisiloxane, in particular sold under the name DC-200 Fluid 1 cst by the company Dow Corning, decamethyltetrasiloxane, in particular sold under the name DC-200 Fluid 1.5 cSt by the company Dow Corning and DC-200 Fluid 5 cst sold by the company Dow Corning, octamethylcyclotetrasiloxane, heptamethylhexyltrisiloxane, heptamethylethyltrisiloxane, heptamethyloctyltrisiloxane and dodecamethylpentasiloxane, and mixtures thereof.

It should be noted that, among the abovementioned oils, linear oils prove to be particularly advantageous.

### Non-Volatile Silicone Oils

According to a second embodiment, the compositions according to the invention comprise at least one non-volatile silicone oil.

The non-volatile silicone oil that can be used in the invention may be chosen from silicone oils having a viscosity at 25° C. of greater than or equal to 9 centistokes (cSt) (9×10⁻⁶ m²/s) and less than 800 000 cSt, preferably between 50 and 600 000 cSt, preferably between 100 and 500 000 cSt. The viscosity of this silicone can be measured according to standard ASTM D-445.

Among these silicone oils, two types of oils can be distinguished according to whether or not they are phenyl oils.

By way of representation of these non-volatile linear silicone oils, mention may be made of polydimethylsiloxanes; alkyl dimethicones; vinylmethyl methicones; and also silicones modified with aliphatic, optionally fluorinated, groups or with functional groups such as hydroxyl, thiol and/or amine groups.

Thus, by way of non-phenyl, non-volatile silicone oils, mention may be made of:
PDMSs comprising alkyl or alkoxy groups which are pendant and/or at the ends of the silicone chain, said groups each having from 2 to 24 carbon atoms,
PDMSs comprising aliphatic groups, or functional groups such as hydroxyl, thiol and/or amine groups,
polyalkylmethylsiloxanes optionally substituted with a fluoro group, such as polymethyltrifluoropropyldimethylsiloxanes,
polyalkylmethylsiloxanes substituted with functional groups such as hydroxyl, thiol and/or amine groups,
polysiloxanes modified with fatty acids, fatty alcohols or polyoxyalkylenes, and mixtures thereof.

According to one embodiment, a composition according to the invention contains at least one non-phenyl linear silicone oil.

The non-phenyl linear silicone oil may in particular be chosen from silicones of formula:

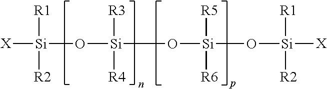

in which:
R1, R2, R5 and R6 are, together or separately, an alkyl radical having 1 to 6 carbon atoms,
R3 and R4 are, together or separately, an alkyl radical having from 1 to 6 carbon atoms, a vinyl radical, an amine radical or a hydroxyl radical,
X is an alkyl radical having from 1 to 6 carbon atoms, a hydroxyl radical or an amine radical,
n and p being integers chosen so as to have a fluid compound.

As non-volatile silicone oil that can be used according to the invention, mention may be made of those for which:
the substituents R1 to R6 and X represent a methyl group, and p and n are such that the viscosity is 500,000 cst, such as the product sold under the name SE30 by the company General Electric, the product sold under the name AK 500000 by the company Wacker, the product sold under the name Mirasil DM 500,000 by the company Bluestar and the product sold under the name Dow Corning 200 Fluid 500,000 cst by the company Dow Corning;
the substituents R1 to R6 and X represent a methyl group, and p and n are such that the viscosity is 60,000 cst, such as the product sold under the name Dow Corning 200 Fluid 60000 CS by the company Dow Corning and the product sold under the name Wacker Belsil DM 60,000 by the company Wacker;
the substituents R1 to R6 and X represent a methyl group, and p and n are such that the viscosity is 350 cst, such as the product sold under the name Dow Corning 200 Fluid 350 CS by the company Dow Corning;
the substituents R1 to R6 represent a methyl group, the group X represents a hydroxyl group, and n and p are such that the viscosity is 700 cst, such as the product sold under the name Baysilone Fluid T0.7 by the company Momentive.

According to one embodiment variant, a composition according to the invention contains at least one phenyl silicone oil.

By way of representation of these non-volatile phenyl silicone oils, mention may be made of:
the phenyl silicone oils corresponding to the following formula:

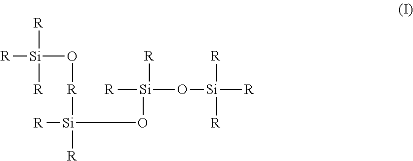

in which the R groups represent, independently of one another, a methyl or a phenyl, with the proviso that at least one R group represents a phenyl. Preferably in this formula, the phenyl silicone oil comprises at least three phenyl groups, for example at least four, at least five or at least six;
the phenyl silicone oils corresponding to the following formula:

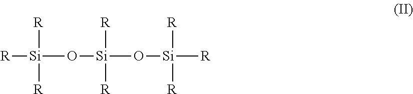

in which the R groups represent, independently of one another, a methyl or a phenyl, with the proviso that at least one R group represents a phenyl. Preferably in this formula, said organopolysiloxane comprises at least three phenyl groups, for example at least four or at least five. Mixtures of the phenyl organopolysiloxanes described above may be used. Mention may, for example, be made of mixtures of triphenyl, tetraphenyl or pentaphenyl organopolysiloxanes;
the phenyl silicone oils corresponding to the following formula:

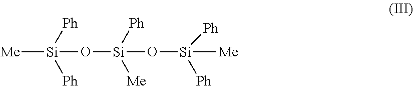

in which Me represents methyl, Ph represents phenyl. Such a phenyl silicone is in particular manufactured by Dow Corning under the reference PH-1555 HR1 or else Dow Corning 555 Cosmetic Fluid (chemical name: 1,3,5-trimethyl 1,1,3,5,5-pentaphenyl trisiloxane; INCI name: trimethyl pentaphenyl trisiloxane). The reference Dow Corning 554 Cosmetic Fluid may also be used;
the phenyl silicone oils corresponding to the following formula (IV):

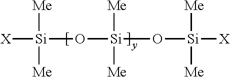

in which Me represents methyl, y is between 1 and 1000, and X represents —CH₂—CH(CH₃)(Ph);
the phenyl silicone oils corresponding to the following formula (V):

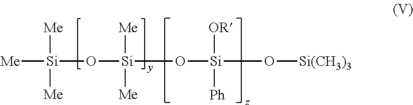

in which Me is methyl and Ph is phenyl, OR′ represents an —OSiMe₃ group and y is 0 or ranges between 1 and 1000, and z ranges between 1 and 1000, such that the compound (V) is a non-volatile oil.

According to a first embodiment, y ranges between 1 and 1000. Use may, for example, be made of trimethyl siloxyphenyl dimethicone, in particular sold under the reference Belsil PDM 1000 by the company Wacker.

According to a second embodiment, y is equal to 0. Use may, for example, be made of phenyl trimethylsiloxy trisiloxane, in particular sold under the reference Dow Corning 556 Cosmetic Grade Fluid;
the phenyl silicone oils corresponding to the following formula (VI) and mixtures thereof:

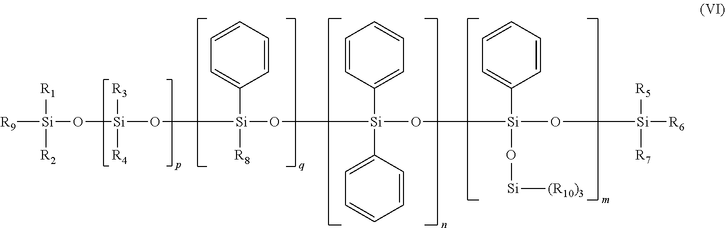

in which:
R₁ to R₁₀, independently of one another, are saturated or unsaturated, linear, cyclic or branched, C₁-C₃₀ hydrocarbon-based radicals,
m, n, p and q are, independently of one another, integers between 0 and 900, with the proviso that the sum ‘m+n+q’ is other than 0.

Preferably, the sum ‘m+n+q’ is between 1 and 100. Preferably, the sum ‘m+n+p+q’ is between 1 and 900, even better still between 1 and 800. Preferably, q is equal to 0;
the phenyl silicone oils corresponding to the following formula (VII), and mixtures thereof:

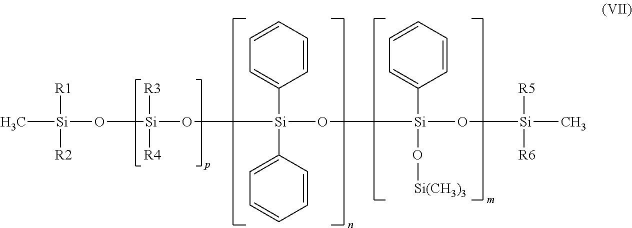

in which:
R1 to R6, independently of one another, are saturated or unsaturated, linear, cyclic or branched, C₁-C₃₀ hydrocarbon-based radicals,
m, n and p are, independently of one another, integers between 0 and 100, with the proviso that the sum ‘n+m’ is between 1 and 100.

Preferably, R1 to R6, independently of one another, represent a saturated, linear or branched, C₁-C₃₀, in particular C₁-C₁₂, hydrocarbon-based radical, and in particular a methyl, ethyl, propyl or butyl radical.

In particular, R1 to R6 may be identical, and in addition may be a methyl radical.

Preferably, it is possible to have m=1 or 2 or 3, and/or n=0 and/or p=0 or 1, in formula (VII);
the phenyl silicone oils corresponding to formula (VIII), and mixtures thereof:

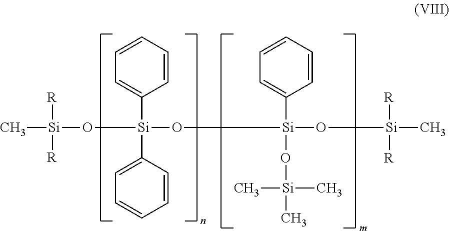

in which:
R is a C₁-C₃₀ alkyl radical, an aryl radical or an aralkyl radical,
n is an integer ranging from 0 to 100, and
m is an integer ranging from 0 to 100, with the proviso that the sum n+m ranges from 1 to 100.

In particular, the R radicals of formula (VIII), and R₁ to R₁₀ defined above, can each represent a linear or branched, saturated or unsaturated, especially C₂-C₂₀, in particular C₃-C₁₆ and more particularly C₄-C₁₀, alkyl radical, or a C₆-C₁₄, in particular C₁₀-C₁₃, monocyclic or polycyclic aryl radical, or an aralkyl radical, the aryl and alkyl residues of which are as defined above.

Preferably, R of formula (VIII) and R₁ to R₁₀ can each represent a methyl, ethyl, propyl, isopropyl, decyl, dodecyl or octadecyl radical, or else a phenyl, tolyl, benzyl or phenethyl radical.

According to one embodiment, use may be made of a phenyl silicone oil of formula (VIII) having a viscosity at 25° C. of between 5 and 1500 mm²/s (i.e. 5 to 1500 cSt), preferably having a viscosity between 5 and 1000 mm²/s (i.e. 5 to 1000 cSt).

As phenyl silicone oil of formula (VIII), use may in particular be made of phenyl trimethicones, such as DC556 from Dow Corning (22.5 cSt) or the Silbione 70663V30 oil from Rhône Poulenc (28 cSt), or diphenyl dimethicones, such as the Belsil oils, in particular Belsil PDM1000 (1000 cSt), Belsil PDM 200 (200 cSt) and Belsil PDM 20 (20 cSt) from Wacker. The values between parentheses represent the viscosities at 25° C.;
phenyl silicone oils corresponding to the following formula, and mixtures thereof:

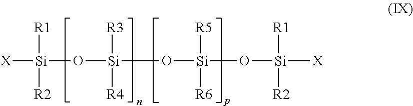

in which:
R1, R2, R5 and R6 are, together or separately, an alkyl radical having 1 to 6 carbon atoms,
R₃ and R₄ are, together or separately, an alkyl radical having from 1 to 6 carbon atoms, or an aryl radical,
X is an alkyl radical having from 1 to 6 carbon atoms, a hydroxyl radical or a vinyl
radical,
n and p being chosen so as to give the oil a weight-average molecular weight of less than 200 000 g/mol, preferably less than 150 000 g/mol and more preferably less than 100 000 g/mol.

The phenyl silicones that are more particularly suitable for the invention are those corresponding to formulae (II) (and in particular formula (III)), and (V), above.

More particularly, the phenyl silicones are chosen more from phenyl trimethicones, phenyl dimethicones, phenyltrimethylsiloxydiphenylsiloxanes, diphenyl dimethicones, diphenylmethyldiphenyltrisiloxanes and 2-phenylethyltrimethylsiloxysilicates, and mixtures thereof.

Preferably, the weight-average molecular weight of the non-volatile phenyl silicone oil according to the invention ranges from 500 to 10 000 g/mol.

### Silicone Gum

According to another embodiment variant, a composition according to the invention contains at least one silicone gum.

The silicone gum that can be used in the invention may be chosen from silicone gums having a viscosity at 25° C. of greater than 800,000 centistokes (cSt) (9×10⁻⁶ m²/s) and in particular between 800,000 and 10,000,000 cSt, preferably between 1,000,000 and 5,000,000 cSt, preferably between 1,000,000 and 2,500,000 cSt. The viscosity of this silicone can be measured according to standard ASTM D-445.

The molecular weight of the silicone gums is generally greater than 350,000 g/mol, between 350,000 and 800,000 g/mol, preferably from 450,000 to 700,000 g/mol

The silicone gum may in particular be chosen from the silicones of the formula:

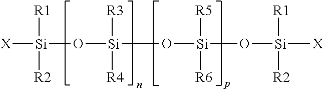

in which:
R1, R2, R5 and R6 are, together or separately, an alkyl radical having 1 to 6 carbon atoms,
R3 and R4 are, together or separately, an alkyl radical having from 1 to 6 carbon atoms, a vinyl radical, an amine radical or a hydroxyl radical,
X is an alkyl radical having from 1 to 6 carbon atoms, a hydroxyl radical or an amine radical,
n and p being integers chosen such that the viscosity of the compound is greater than 800,000 cSt.

As silicone gum that can be used according to the invention, mention may be made of those for which:
the substituents R1 to R6 represent a methyl group, the group X represents a methyl group, and n and p are such that the molecular weight of the polymer is 600 000 g/mol, such as the product sold under the name Mirasil C-DPDM by the company Bluestar;
the substituents R1 to R6 represent a methyl group, the group X represents a hydroxyl group, and n and p are such that the molecular weight of the polymer is 600 000 g/mol, such as the product sold under the name SGM 36 by the company Dow Corning;
dimethicones of the (polydimethylsiloxane)(methylvinylsiloxane) type, such as SE63 sold by GE Bayer Silicones, poly(dimethylsiloxane)(diphenyl) (methylvinylsiloxane) copolymers, and mixtures thereof.

Advantageously, a composition according to the invention may comprise from 0.1% to 60% by weight of silicone compound(s) according to the invention, relative to the total weight of the composition.

In particular, it may comprise from 0.2% to 50% by weight of silicone compound(s) according to the invention, relative to the total weight of the composition.

More particularly, it may comprise from 0.5% to 40% by weight of silicone compound(s) according to the invention, relative to the total weight of the composition.

### Solid Fatty Substances

A composition according to the invention may also comprise at least one solid fatty substance, in particular chosen from waxes and/or pasty fatty substances.

Preferably, the amount of pasty substance in the makeup and/or care composition according to the invention is between 0.5% and 50% by weight, in particular 1% to 40% by weight, or even 2% to 30% by weight, relative to the total weight of the composition.

### Waxes

According to a first embodiment, the composition is free of wax.

According to a second embodiment, the composition comprises at least one wax.

According to this embodiment, the amount of wax(es) in the makeup and/or care composition according to the invention can preferably range from 0.1% to 70% by weight, relative to the total weight of the composition, preferably from 1% to 40% by weight, and better still from 5% to 30% by weight.

Preferably, in particular in the case of makeup compositions for the skin and/or the lips, the wax content is between 0.5% and 30% by weight, in particular 1% to 20% by weight, or even 2% to 15% by weight, relative to the total weight of the composition.

The term “wax” is intended to mean a lipophilic compound that is solid at ambient temperature (25° C.), with a solid/liquid reversible change of state, having a melting point of greater than or equal to 30° C., which may be up to 200° C. The waxes may be chosen from waxes of animal, plant, mineral or synthetic origin and mixtures thereof. Mention may in particular be made of hydrocarbon-based waxes, for instance beeswax, lanolin wax and Chinese insect waxes; rice bran wax, Carnauba wax, Candelilla wax, Ouricury wax, Alfalfa wax, berry wax, shellac wax, Japan wax and sumach wax; montan wax, orange wax and lemon wax, microcrystalline waxes, paraffins and ozokerite; polyethylene waxes, waxes obtained by Fischer-Tropsch synthesis and waxy copolymers, and also esters thereof. Mention may also be made of waxes obtained by catalytic hydrogenation of animal or plant oils having linear or branched C₈-C₃₂ fatty chains. Among these, mention may in particular be made of hydrogenated sunflower oil, hydrogenated castor oil, hydrogenated coconut oil, hydrogenated lanolin oil and bis(1,1,1-trimethylolpropane) tetrastearate.

Mention may also be made of silicone waxes and of fluoro waxes. Use may also be made of waxes obtained by hydrogenation of castor oil esterified with cetyl alcohol.

Advantageously, a composition according to the invention may comprise at least one wax which is in particular hydrocarbon-based.

### Pasty Fatty Substances

According to a first embodiment, the composition is free of pasty fatty substances.

According to a second embodiment, the composition comprises at least one pasty fatty substance. According to this embodiment, the amount of pasty fatty substance in the makeup and/or care composition according to the invention is preferably between 0.5% and 30% by weight, in particular 1% to 20% by weight, or even 2% to 15% by weight, relative to the total weight of the composition.

The term “pasty fatty substance” is intended to mean a lipophilic fatty compound with a reversible solid/liquid change of state and comprising a liquid fraction and a solid fraction at a temperature of 23° C. The pasty compound preferably has a hardness at 20° C. ranging from 0.001 to 0.5 MPa, preferably from 0.002 to 0.4 MPa. The pasty compound is preferably chosen from synthetic compounds and compounds of plant origin. A pasty compound can be obtained by synthesis from starting products of plant origin. Mention may in particular be made, alone or as a mixture, of:
lanolin and derivatives thereof, such as lanolin alcohol, oxyethylenated lanolins, acetylated lanolin, lanolin esters such as isopropyl lanolate, or oxypropylenated lanolins,
polymeric or non-polymeric silicone compounds having a viscosity of greater than 10,000,000 cSt at 25° C., for instance high-molecular-weight polydimethylsiloxanes, polydimethylsiloxanes with side chains of the alkyl or alkoxy type having from 8 to 24 carbon atoms, in particular stearyl dimethicones,
polymeric or non-polymeric fluoro compounds,
   vinyl polymers, in particular olefin homopolymers; olefinic copolymers; hydrogenated diene homopolymers and copolymers; linear or branched homopolymer or copolymer oligomers of alkyl (meth)acrylates preferably containing a C₈-C₃₀ alkyl group; homopolymer and copolymer oligomers of vinyl esters containing C₈-C₃₀ alkyl groups; homopolymer and copolymer oligomers of vinyl ethers containing C₈-C₃₀ alkyl groups,
liposoluble polyethers resulting from polyetherification between one or more C₂-C₁₀₀, preferably C₂-C₅₀, diols; and in particular copolymers of ethylene oxide and/or of propylene oxide with C₆-C₃₀ long-chain alkylene oxides, more preferably such that the weight ratio of the ethylene oxide and/or propylene oxide to the alkylene oxides in the copolymer is from 5:95 to 70:30,
polyol ethers chosen from ethers of pentaerythritol and of polyalkylene glycol, ethers of fatty alcohol and of sugar, and mixtures thereof, the ether of pentaerythritol and of polyethylene glycol comprising 5 oxyethylene (5 OE) units (CTFA name: PEG-5 pentaerythityl ether), and the ether of pentaerythritol and of polypropylene glycol comprising 5 oxypropylene (5 OP) units (CTFA name: PPG-5 pentaerythrityl ether), and mixtures thereof;
esters and polyesters; and in particular (i) the esters of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic acid and isostearic acid, and 12-hydroxystearic acid; (ii) phytosterol esters, (iii) pentaerythritol esters; (iv) esters formed from at least one alcohol, at least one of the alcohols being a Guerbet alcohol, and of a diacid dimer formed from at least one unsaturated fatty acid; (v) non-crosslinked polyesters resulting from polycondensation between a linear or branched C₄-C₅₀ dicarboxylic acid or polycarboxylic acid and a C₂-C₅₀ diol or polyol, (vi) polyesters which result from esterification, with a polycarboxylic acid, of an ester of an aliphatic hydroxycarboxylic acid; (vii) aliphatic esters of an ester resulting from the esterification of an aliphatic hydroxycarboxylic acid ester with an aliphatic carboxylic acid containing in particular 4 to 30 carbon atoms. The aliphatic hydroxycarboxylic acid ester is advantageously derived from a hydroxylated aliphatic carboxylic acid containing 2 to 40 carbon atoms and 1 to 20 hydroxyl groups; (viii) aliphatic esters of an ester, chosen from the ester resulting from the esterification reaction of hydrogenated castor oil with isostearic acid (hydrogenated castor oil monoisostearate, diisostearate or triisostearate).

The pasty compound may also be of plant origin. Mention may in particular be made of isomerized jojoba oil, such as trans-isomerized, partially hydrogenated jojoba oil; orange wax, shea butter, partially hydrogenated olive oil, cocoa butter and mango oil.

### Dyestuffs

The composition according to the invention may also comprise one or more dyestuffs chosen from pulverulent compounds, for instance pigments, pearlescent agents and glitter flakes, and/or liposoluble or water-soluble dyes. The dyestuffs, in particular pulverulent dyestuffs, may be present in the composition in a content of from 0.01% to 50% by weight, relative to the weight of the composition, preferably from 0.1% to 40% by weight, or even from 1% to 30% by weight.

The term “pigments” should be understood to mean white or coloured, mineral or organic particles of any shape, which are insoluble in the physiological medium, and which are intended to colour the composition. The term “pearlescent agents” should be understood to mean iridescent particles of any shape, in particular produced by certain molluscs in their shell, or alternatively synthesized.

The pigments may be white or coloured, mineral and/or organic, and interference or non-interference. Among the mineral pigments, mention may be made of titanium dioxide, optionally surface-treated, zirconium oxide or cerium oxide, and also iron oxide, chromium oxide, manganese violet, ultramarine blue, chromium hydrate and ferric blue. Among the organic pigments, mention may be made of carbon black, pigments of D & C type, and lakes based on cochineal carmine, or on barium, strontium, calcium or aluminium.

The pearlescent pigments may be chosen from white pearlescent pigments such as mica coated with titanium or with bismuth oxychloride, coloured pearlescent pigments such as titanium mica with iron oxides, titanium mica with in particular ferric blue or with chromium oxide, titanium mica with an organic pigment of the abovementioned type, and also pearlescent pigments based on bismuth oxychloride.

The water-soluble dyes are, for example, beetroot juice and methylene blue and can represent 0.01% to 6% of the total weight of the composition.

Preferably, in particular in the case of a makeup composition, the composition comprises at least one dyestuff.

The dyestuff is in particular chosen from organic or inorganic dyestuffs, in particular of the type of pigments or pearlescent agents conventionally used in cosmetic compositions, liposoluble or water-soluble dyes, materials with a specific optical effect, and mixtures thereof.

Preferably, the amount of dyestuff(s) in a composition according to the invention, in particular in the case of a makeup composition, is between 0.01% and 40% by weight, in particular 0.1% and 30% by weight, or even 1% and 20% by weight, of the total weight of the composition.

### Fillers

According to another particular embodiment, a composition according to the invention, in particular a care and/or makeup composition for the skin and/or the lips, comprises at least one organic or inorganic filler. Preferably, it will be a sebum-absorbing filler in the case of a makeup composition for the skin or a silicone filler, in particular in the case of a makeup composition for the lips. The presence of such a filler makes it possible in particular to reduce the tack of the deposit when it is applied and while wearing it.

This particular embodiment can in particular make it possible to obtain compositions, in particular makeup compositions, of which the deposit on keratin materials, and in particular the lips and/or the skin, is uniform and/or not very tacky or not at all tacky. Such a deposit can in particular provide a feeling of comfort while being worn (softness, property of glidance of the deposit formed).

In addition, such a composition may have transfer resistance properties and also properties of colour fastness of the deposit (no fragility or fragmentation of the deposit, which remains uniform, and resistant to rubbing), and of staying power with respect to grease.

As specified above, a composition according to the invention may also comprise at least one organic or inorganic filler.

Thus, a composition may comprise from 0.01% to 35% by weight, preferably 0.1% to 20% by weight of filler(s), relative to its total weight.

By way of illustration of these fillers, mention may be made of talc, mica, silica, kaolin, calcium carbonate, barium sulphate, nylon powders (in particular Orgasol) and polyethylene powders, teflon, starch, boron nitride, copolymer microspheres such as Expancel (Nobel Industrie); and also mixtures thereof.

According to one embodiment variant, a composition according to the invention contains at least one filler capable of absorbing an oil.

In particular, a composition according to the invention comprises at least one filler which has a capacity to absorb and/or adsorb an oil or a liquid fatty substance such as, for example, sebum (of the skin).

This oil-absorbing filler may also advantageously have a BET specific surface area greater than or equal to 300 m²/g, preferably greater than 500 m²/g, and preferentially greater than 600 m²/g, and in particular less than 1500 m²/g.

The “BET specific surface area” is determined according to the BET (Brunauer-Emmet-Teller) method described in “The journal of the American Chemical Society”, vol. 60, page 309, February 1938 and corresponding to international standard ISO 5794/1 (annex D). The BET specific surface area corresponds to the total specific surface area (therefore including micropores) of the powder.

The filler under consideration according to the invention is thus characterized in that it has an oil uptake of greater than or equal to 1 ml/g, in particular ranging from 1 ml/g to 20 ml/g, or even ranging from 1.5 ml/g to 15 ml/g. Preferably, it has an oil uptake of greater than or equal to 2 ml/g, in particular ranging from 2 ml/g to ml/g, or even ranging from 2 ml/g to 15 ml/g.

This oil uptake, which corresponds to the amount of oil absorbed and/or adsorbed by the filler, can be characterized by measuring the wet point according to the method described hereinafter.

### Method for Measuring Oil Uptake of a Pulverulent Material:

The oil uptake of a powder is measured according to the method for determining oil uptake of a powder described in standard NF T 30-022. It corresponds to the amount of oil adsorbed onto the available surface of the pulverulent material by measurement of the wet point.

An amount m (in grams) of powder of between approximately 0.5 g and 5 g (the amount depends on the density of the powder) is placed on a glass plate and then isononyl isononanoate is added dropwise.

After the addition of 4 to 5 drops of isononyl isononanoate, the isononyl isononanoate is incorporated into the filler using a spatula and isononyl isononanoate continues to be added until the formation of conglomerates of isononyl isononanoate and of powder. From this moment on, isononyl isononanoate is added one drop at a time and the mixture is then triturated with the spatula. The addition of isononyl isononanoate is stopped when a smooth firm paste is obtained. It should be possible to spread this paste over the glass plate without there being any cracks or any formation of lumps. The volume Vs (expressed in ml) of isononyl isononanoate used is then noted.

The oil uptake corresponds to the Vs/m ratio.

This oil-absorbing filler may be a mineral powder or an organic powder; it may be chosen from silica, polyamide (Nylon®) powders, powders of acrylic polymers, in particular of polymethyl methacrylate, or of polymethyl methacrylate/ethylene glycol dimethacrylate, of polyallyl methacrylate/ethylene glycol dimethacrylate, of ethylene glycol dimethacrylate/lauryl methacrylate copolymer; silicone elastomer powders, in particular obtained by polymerization of organopolysiloxane having at least two hydrogen atoms each bonded to a silicon atom and of an organopolysiloxane comprising at least two ethylenically unsaturated groups (in particular two vinyl groups) in the presence of a platinum catalyst.

The oil-absorbing filler may be a powder coated with a hydrophobic treatment agent.

Examples of fillers having an oil uptake of greater than or equal to 1.5 ml/g are described below, with their oil uptake value measured according to the protocol defined above.

As silica powders, mention may be made of:
porous silica microspheres, in particular those sold under the name Sunsphere® H53, Sunsphere® H33 (oil uptake equal to 3.70 ml/g) by the company Asahi Glass; MSS-500-3H by the company Kobo; Silica Beads SB-700 by the company Myoshi;
polydimethylsiloxane-coated amorphous silica microspheres, in particular those sold under the name SA Sunsphere® H 33 (oil uptake equal to 2.43 ml/g);
silica silylate powders, in particular those sold under the name Dow Corning VM-2270 Aerogel Fine Particles by the company Dow Corning (oil uptake equal to 10.40 ml/g);
hollow amorphous silica particles, in particular those sold under the name Silica Shells by the company Kobo (oil uptake equal to 5.50 ml/g);
precipitated silica powders surface-treated with a mineral wax, such as precipitated silica treated with a polyethylene wax, and in particular those sold under the name Acematt OR412 by the company Evonik Degussa (oil uptake equal to 3.98 ml/g).

As acrylic polymer powders, mention may be made of:
porous spheres of polymethyl methacrylate/ethylene glycol dimethacrylate, sold under the name Microsponge 5640 by the company Cardinal Health technologies (oil uptake equal to 1.55 ml/g), Ganzpearl® GMP-0820 by the company Ganz Chemical;
powders of ethylene glycol dimethacrylate/lauryl methacrylate copolymer, in particular those sold under the name Polytrap® 6603 from the company Dow Corning (oil uptake equal to 6.56 ml/g);
polymethyl methacrylate powders sold under the name Covabead® LH85 by the company Wackherr;
powders of polyallyl methacrylate/ethylene glycol dimethacrylate sold under the name Poly-Pore® L200, Poly-Pore® E200 by the company Amcol.

As polyamide powders, mention may be made of:
the nylon powder sold under the name Orgasol® 4000 by the company Atochem;
nylon-6 powder, in particular the product sold under the name POMP610 by the company Ube Industries (oil uptake equal to 2.02 ml/g).

As perlite powder mention may in particular be made of the product sold under the name Optimat 1430 OR by the company World Minerals (oil uptake equal to 2.4 ml/g).

As magnesium carbonate powder, mention may in particular be made of the product sold under the name Tipo Carbomagel by the company Buschle & Lepper (oil uptake equal to 2.14 ml/g).

The oil-absorbing filler which is particularly preferred is a silica powder and more particularly a silica powder having an oil uptake at least equal to 3.70 ml/g, and in particular those sold under the name Sunsphere® H 33 by the company Asahi Glass, and under the name Dow Corning VM-2270 Aerogel Fine Particles by the company Dow Corning.

The filler(s) in particular capable of absorbing an oil may be present in a composition according to the invention in a content ranging from 0.5% to 40% by weight, preferably from 1% to 20% by weight, and better still from 1% to 15% by weight, relative to the total weight of the composition.

A composition according to the invention may use at least one filler and at least one supramolecular polymer in a polymer(s)/oil-absorbing filler(s) weight ratio of greater than 1, preferably greater than 1.5, and even better still greater than 2.

According to one embodiment variant, a composition according to the invention contains at least one filler having an oil uptake of greater than or equal to 1.5 ml/g.

### Silicone Filler

The compositions according to the invention may comprise at least one silicone filler.

The silicone filler may be chosen from silicone-resin-coated organopolysiloxane powders and polymethylsilsesquioxane powders, and mixtures thereof.

The organopolysiloxane powder may in particular be coated with silsesquioxane resin, as described, for example, in U.S. Pat. No. 5,538,793. Such elastomer powders are sold under the names KSP-100, KSP-101, KSP-102, KSP-103, KSP-104 and KSP-105 by the company Shin Etsu, and have the INCI name: vinyl dimethicone/methicone silsesquioxane crosspolymer.

As polymethylsilsesquioxane powder, mention may in particular be made of silicone resin microbeads such as those sold under the name Tospearl by the company Momentive Performance Materials, and in particular under the reference Tospearl 145 A; and mixtures thereof.

In particular, the composition according to the invention may comprise a silicone filler chosen from silicone-resin-coated organopolysiloxane powders and polymethylsilsesquioxane powders.

### Silicone Elastomer

According to another embodiment variant, a composition according to the invention, in particular a makeup composition for the skin and/or the lips, may comprise at least one silicone elastomer, otherwise known as organopolysiloxane elastomer.

The term “organopolysiloxane elastomer” is intended to mean a supple, deformable organopolysiloxane having viscoelastic properties and in particular the consistency of a sponge or of a supple sphere. Its modulus of elasticity is such that this material withstands deformation and has a limited capacity for extension and contraction. This material is capable of retaining its original shape after having been stretched.

It is more particularly a crosslinked organopolysiloxane elastomer. Preferably, the organopolysiloxane elastomer is obtained by crosslinking addition reaction (A) of diorganopolysiloxane containing at least two hydrogens each bonded to a silicon, and (B) of diorganopolysiloxane having at least two ethylenically unsaturated groups bonded to silicon, in particular in the presence (C) of a platinum catalyst, as described, for example, in application EP-A-295886.

In particular, the organopolysiloxane elastomer can be obtained by reaction of dimethylpolysiloxane containing dimethylvinylsiloxy end groups and of methylhydrogenopolysiloxane containing trimethylsiloxy end groups, in the presence of a platinum catalyst.

Compound (A) may in particular be chosen from methylhydrogenopolysiloxanes containing trimethylsiloxy end groups, dimethylsiloxane-methylhydrogenosiloxane copolymers containing trimethylsiloxy end groups and dimethylsiloxane-methylhydrogenosiloxane cyclic copolymers.

The organopolysiloxanes (B) may in particular be chosen from methylvinylpolysiloxanes, methylvinylsiloxane-dimethylsiloxane copolymers, dimethylpolysiloxanes containing dimethylvinylsiloxy end groups, dimethylsiloxane-methylphenyl-siloxane copolymers containing dimethylvinylsiloxy end groups, dimethylsiloxane-diphenylsiloxane-methylvinylsiloxane copolymers containing dimethylvinylsiloxy end groups, dimethylsiloxane-methylvinylsiloxane copolymers containing trimethylsiloxy end groups, dimethylsiloxane-methylphenyl-siloxane-methylvinylsiloxane copolymers containing trimethylsiloxy end groups, methyl(3,3,3-trifluoropropyl)polysiloxane containing dimethylvinylsiloxy end groups, and dimethylsiloxane-methyl(3,3,3-trifluoropropyl)siloxane copolymers containing dimethylvinylsiloxy end groups.

It is advantageous for compound (A) to be added in an amount such that the molecular ratio between the total amount of hydrogen atoms bonded to silicon atoms in compound (A) and the total amount of all the ethylenically unsaturated groups in compound (B) is within the range of from 1.5/1 to 20/1.

Compound (C) is the catalyst for the crosslinking reaction, and is in particular chloroplatinic acid, chloroplatinic acid-olefin complexes, chloroplatinic acidalkenylsiloxane complexes, chloroplatinic acid-diketone complexes, platinum black or platinum on a support.

The catalyst (C) is preferably added at from 0.1 to 1000 parts by weight, better still 1 to 100 parts by weight, as plain platinum metal per 1000 parts by weight of the total amount of compounds (A) and (B).

The elastomer is advantageously a non-emulsifying elastomer.

The term “non-emulsifying” defines organopolysiloxane elastomers that do not contain any hydrophilic chains, and in particular that do not contain any polyoxyalkylene units (especially polyoxyethylene or polyoxypropylene), or any polyglyceryl units.

The organopolysiloxane elastomer particles are conveyed in the form of a gel consisting of an elastomeric organopolysiloxane included in at least one hydrocarbon-based oil and/or one silicone oil. In these gels, the organopolysiloxane particles are often non-spherical particles.

Non-emulsifying elastomers are in particular described in patents EP 242 219, EP 285 886 and EP 765 656 and in application JP-A-61-194009, the content of which is incorporated by way of reference.

Spherical non-emulsifying elastomers that may be used include those sold under the names DC 9040, DC 9041, DC 9509, DC 9505 and DC 9506 by the company Dow Corning.

Use may also be made, in the compositions according to the invention, of organopolysiloxane elastomers with an MQ group, such as those sold by the company Wacker under the names Belsil RG100, Belsil RPG33 and preferentially RG80. The elastomer may also be an emulsifying elastomer.

The term “emulsifying organopolysiloxane elastomer” is intended to mean an organopolysiloxane elastomer comprising at least one hydrophilic chain, such as polyoxyalkylenated organopolysiloxane elastomers and polyglycerolated silicone elastomers.

The emulsifying organopolysiloxane elastomer may be chosen from polyoxyalkylenated organopolysiloxane elastomers.

The polyoxyalkylenated organopolysiloxane elastomer is a crosslinked organopolysiloxane elastomer which can be obtained by means of a crosslinking addition reaction of diorganopolysiloxane containing at least one hydrogen bonded to silicon and of a polyoxyalkylene containing at least two ethylenically unsaturated groups. Advantageously, the polyoxyalkylenated organopolysiloxane elastomers may be formed from divinyl compounds, in particular polyoxyalkylenes containing at least two vinyl groups, which react with Si—H bonds of a polysiloxane.

Polyoxyalkylenated elastomers are in particular described in U.S. Pat. No. 5,236,986, U.S. Pat. No. 5,412,004, U.S. Pat. No. 5,837,793 and U.S. Pat. No. 5,811,487, the content of which is incorporated by way of reference.

Polyoxyalkylenated organopolysiloxane elastomers that can be used include those sold under the names KSG-21, KSG-20, KSG-30, KSG-31, KSG-32, KSG-33, KSG-210, KSG-310, KSG-320, KSG-330 and KSG-340 by the company Shin Etsu, and DC9010 and DC9011 by the company Dow Corning.

Emulsifying organopolysiloxane elastomer may also be chosen from polyglycerolated organopolysiloxane elastomers.

The polyglycerolated organopolysiloxane elastomer according to the invention is an organopolysiloxane elastomer which can be obtained by means of a crosslinking addition reaction of diorganopolysiloxane containing at least one hydrogen bonded to silicon and of polyglycerolated compounds having ethylenically unsaturated groups, in particular in the presence of a platinum catalyst.

The polyglycerolated organopolysiloxane elastomer according to the invention is conveyed in the form of a gel in at least one hydrocarbon-based oil and/or one silicone oil. In these gels, the polyglycerolated elastomer is often in the form of non-spherical particles.

Polyglycerolated organopolysiloxane elastomers that may be used include those sold under the names KSG-710, KSG-810, KSG-820, KSG-830 and KSG-840 by the company Shin Etsu.

Non-emulsifying elastomers that can more particularly be used include those sold under the names KSG-6, KSG-15, KSG-16, KSG-18, KSG-41, KSG-42, KSG-43 and KSG-44 by the company Shin Etsu, DC9040 and DC9041 by the company Dow Corning and SFE 839 by the company General Electric.

Emulsifying elastomers that can more particularly be used include those sold under the names KSG-31, KSG-32, KSG-33, KSG-210 and KSG-710 by the company Shin Etsu.

Advantageously, the organopolysiloxane elastomer under consideration according to the invention is chosen from spherical, non-emulsifying organopolysiloxane elastomers, polyglycerolated organopolysiloxane elastomers and polyoxyalkylenated organopolysiloxane elastomers.

It is more particularly a polyoxyalkylenated organopolysiloxane elastomer.

The composition according to the invention may comprise an organopolysiloxane elastomer, alone or as a mixture, in a content ranging from 0.1% to 20% by weight, preferably from 0.2% to 15% by weight, and even more preferably from 0.5% to 12% by weight.

The composition may also comprise other ingredients commonly used in cosmetic compositions. Such ingredients may be chosen from water, hydrophilic solvents, antioxidants, fragrances, essential oils, preservatives, cosmetic active agents, moisturizers, vitamins, ceramides, sunscreens, surfactants, gelling agents, thickeners, spreading agents, wetting agents, dispersants, antifoams, neutralizing agents, stabilizers, polymers and in particular film-forming polymers, and mixtures thereof. Of course, those skilled in the art will take care to select this or these optional additional ingredient(s) and the amount thereof in such a way that the advantageous properties of the composition are not, or not substantially, impaired by the addition envisaged.

In particular, by way of film-forming polymers, use may especially be made of a film-forming polymer in particular chosen from polyamide silicone block polymers, block ethylenic polymers, vinyl polymers comprising at least one carbosiloxane dendrimer derivative, copolymers comprising carboxylate groups and polydimethylsiloxane groups, silicone resins and lipodispersible polymers in the form of a non-aqueous dispersion of particles of polymers and mixtures thereof.

Preferably, the film-forming polymer may be chosen from the group comprising:
a block ethylenic copolymer (also called block ethylenic polymer), containing at least one first block having a glass transition temperature (Tg) of greater than or equal to 40° C. and being totally or partially derived from one or more first monomers, which are such that the homopolymer prepared from these monomers has a glass transition temperature of greater than or equal to 40° C., and at least one second block having a glass transition temperature of less than or equal to 20° C. and being totally or partially derived from one or more second monomers, which are such that the homopolymer prepared from these monomers has a glass transition temperature of less than or equal to 20° C., said first block and said second block being connected to one another via a random intermediate segment comprising at least one of said first constituent monomers of the first block and at least one of said second constituent monomers of the second block, and said block copolymer having a polydispersity index I of greater than 2, as described in FR 0953625, incorporated by way of reference,
a vinyl polymer comprising at least one unit derived from a carbosiloxane dendrimer, as described in applications WO03/045337 and EP 963 751 from the company Dow Corning,
a dispersion of particles of acrylic or vinyl radical homopolymer or copolymer, dispersed in said liquid fatty phase, as described in application WO 04/055081,
polyamide silicone block copolymers (also known as silicone polyamides) comprising at least one unit of formula (III) or (IV):

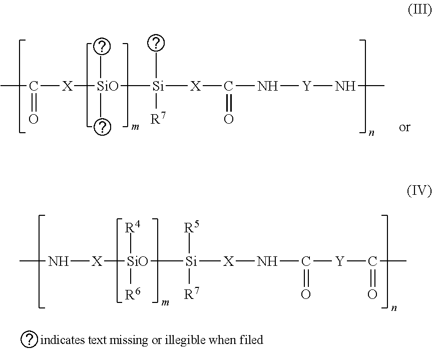

in which:
1) R⁴, R⁵, R⁶ and R⁷, which may be identical or different, represent a group chosen from:
linear, branched or cyclic, saturated or unsaturated, C₁ to C₄₀ hydrocarbon-based groups which can contain, in their chain, one or more oxygen, sulphur and/or nitrogen atoms, and which can be partially or totally substituted with fluorine atoms,
C₆ to C₁₀ aryl groups, optionally substituted with one or more C₁ to C₄ alkyl groups,
polyorganosiloxane chains optionally containing one or more oxygen, sulphur and/or nitrogen atoms,
   2) the X, which may be identical or different, represent a linear or branched C₁ to C₃₀ alkylenediyl group, which can contain, in its chain, one or more oxygen and/or nitrogen atoms,
   3) Y is a C₁-C₅₀, saturated or unsaturated, arylalkylene, alkylarylene, cycloalkylene, arylene, linear or branched alkylene divalent group which can comprise one or more oxygen, sulphur and/or nitrogen atoms, and/or can bear, as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups, or
   4) Y represents a group corresponding to the formula:

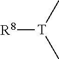

in which:
T represents a linear or branched, saturated or unsaturated, C₃ to C₂₄ trivalent or tetravalent hydrocarbon-based group optionally substituted with a polyorganosiloxane chain, and which can contain one or more atoms chosen from O, N and S, or T represents a trivalent atom chosen from N, P and Al, and
R⁸ represents a linear or branched C₁ to C₅₀ alkyl group, or a polyorganosiloxane chain, which can comprise one or more ester, amide, urethane, thiocarbamate, urea, thiourea and/or sulphonamide groups, which may possibly be linked to another chain of the polymer,
   n is an integer ranging from 2 to 500, preferably from 2 to 200, and m is an integer ranging from 50 to 1000, preferably from 50 to 700 and even better still from 50 to 200; as described in application PCT/FR2009/052388, incorporated by way of reference,
silicone resins, in particular chosen from polymethylsilsesquioxanes, siloxysilicate resins, in particular trimethylsiloxysilicate resins, as described in application FR0954344, incorporated by way of reference,
a copolymer comprising carboxylate groups and polydimethylsiloxane groups, in particular chosen from copolymers of acrylic acid and of stearyl acrylate comprising polydimethylsiloxane grafts, copolymers of stearyl methacrylate comprising polydimethylsiloxane grafts, copolymers of acrylic acid and of stearyl methacrylate comprising polydimethylsiloxane grafts, copolymers of methyl methacrylate, of butyl methacrylate, of 2-ethylhexyl acrylate and of stearyl methacrylate comprising polydimethylsiloxane grafts,
and mixtures thereof.

The presence of a film-forming polymer in the compositions according to the invention may in particular make it possible to improve the grease resistance (staying power with respect to grease) of the deposits formed with these compositions on keratin materials, in particular such as the skin and/or the lips, improves the staying power, in particular the colour fastness, of the deposit (resistance to rubbing), and makes it possible to obtain a deposit which is not very tacky or not at all tacky.

The compositions according to the invention may be in any form which is acceptable and customary for a cosmetic composition. They may therefore be in the form of a suspension, a dispersion, in particular of oil in water by virtue of vesicles, or water in oil; an aqueous, organic or oily solution which is optionally thickened or even gelled; an oil-in-water, water-in-oil or multiple emulsion; a gel, in particular an aqueous, oily or emulsified gel; a foam; a dispersion of vesicles, in particular lipid vesicles; a two-phase or multiphase lotion; a spray; a lotion, a cream, an ointment, a soft paste, a salve, a solid which has been cast or moulded, in particular as a stick or in a dish, or a compacted solid. Those skilled in the art may select the appropriate galenical form, and also the method for preparing it, on the basis of their general knowledge, taking into account firstly the nature of the constituents used, in particular their solubility in the support, and secondly the intended use of the composition.

### Aqueous Phase

A composition according to the invention may also comprise an aqueous phase, which may represent 1% to 80% by weight, in particular 2% to 70% by weight, or even 3% to 60% by weight, of the total weight of the composition. This aqueous phase may consist essentially of water, or may comprise a mixture of water and of a water-miscible solvent (miscibility in water greater than 50% by weight at 25° C.) in particular chosen from monoalcohols containing 1 to 5 carbon atoms, such as ethanol or isopropanol, glycols containing 2 to 8 carbon atoms, such as propylene glycol, ethylene glycol, 1,3-butylene glycol or dipropylene glycol, C₃-C₄ ketones, C₂-C₄ aldehydes, and mixtures thereof.

However, as specified above, the compositions according to the invention are advantageously anhydrous.

According to one particular embodiment, in particular in the case of a composition dedicated to lip or facial care and/or makeup, the composition used according to the invention is anhydrous or contains less than 3% by weight of water, preferably less than 1% by weight of water, relative to the total weight of the composition. In the case of a makeup composition for the lips, the composition is preferably anhydrous.

The term “anhydrous” is intended to mean in particular that water is preferably not intentionally added to the composition, but may be present in trace amounts in the various compounds used in the composition.

The compositions according to the invention can be used for caring for or making up keratin materials such as the skin, the eyelashes, the eyebrows, the nails, the lips, the hair, and more particularly for making up the lips, the hair, the eyelashes and/or the face.

They can therefore be in the form of a care and/or makeup product for the skin of the body or of the face, the lips, the eyelashes, the eyebrows, the hair or the nails; of an antisun or self-tanning product; or of a hair product; they are advantageously in the form of a makeup composition, in particular a mascara, eyeliner, lipstick, lip gloss, face powder, eye shadow, foundation, nail varnish or hair mascara composition.

In particular, in the case of a lipstick, the composition may be in liquid (gloss) or solid form, for example such as a lipstick in the form of a stick or cast in a dish.

Generally, the compositions according to the invention may be in solid or liquid form at 20° C.

For the purpose of the invention, the term “solid” characterizes the state of the composition at a temperature of 20° C. In particular, a solid composition according to the invention has, at a temperature of 20° C. and at atmospheric pressure (760 mmHg), a hardness of greater than 30 Nm⁻¹, preferably greater than 40 Nm⁻¹.

### Protocol for Measuring the Hardness:

The hardness of a composition, in particular such as a stick of a lipstick, is measured according to the following protocol:

The stick is stored at 20° C. for 24 hours before measuring the hardness.

The hardness may be measured at 20° C. via the “cheese wire” method, which consists in transversely cutting a wand of product, which is preferably a circular cylinder, by means of a rigid tungsten wire 250 μm in diameter, by moving the wire relative to the stick at a speed of 100 mm/min.

The hardness of the samples of compositions of the invention, expressed in Nm⁻¹, is measured by means of a DFGS2 dynamometer sold by the company Indelco-Chatillon.

The measurement is reproduced three times and then averaged. The average of the three values read using the dynamometer mentioned above, denoted Y, is given in grams. This average is converted to newtons and then divided by L which represents the longest distance through which the wire passes. In the case of a cylindrical wand, L is equal to the diameter (in metres).

The hardness is converted into Nm⁻¹ by means of the equation below:

(Y×10⁻³×9.8)/L

For a measurement at a different temperature, the stick is stored for 24 hours at this new temperature before the measurement.

According to this method of measurement, a solid composition according to the invention has a hardness at 20° C. of greater than or equal to 30 Nm⁻¹, preferably greater than 40 Nm⁻¹, preferably greater than 50 Nm⁻¹.

Preferably, the composition according to the invention has in particular a hardness at 20° C. of less than 500 Nm⁻¹, in particular less than 400 Nm⁻¹, preferably less than 300 Nm⁻¹.

In particular, a composition of which the hardness is greater than 30 Nm⁻¹ is a “solid” composition at 20° C. and at atmospheric pressure (760 mmHg).

The compositions according to the invention find their most particular use in the field of lipsticks and foundations, compositions which are particularly sensitive to grease (sebum and food grease).

Another subject of the invention is a cosmetic treatment process for keratin materials, in particular the skin of the body or of the face, the lips, the nails, the eyelashes, and/or the hair, comprising the application, to said materials, of a cosmetic composition as defined above.

This process according to the invention makes it possible in particular to care for or make up said keratin materials, in particular the lips, the hair, the face and/or the eyelashes, by applying a composition, in particular lipstick, foundation, or mascara for the eyelashes or hair according to the invention.

A composition according to the invention may be in the form of a makeup composition for the skin and/or the lips, in particular for the skin of the face or of the body; it may be a product for the complexion, such as a foundation, a face powder or an eye shadow; a lip product, such as a lipstick or a lipcare product; a concealer product; a blusher, an eyeliner; a lip or eye pencil; a body makeup product; a gloss (lip gloss).

According to a first advantageous embodiment of the invention, the composition according to the invention is dedicated to making up the skin and it is then more particularly a foundation, a face powder or an eye shadow, or a body makeup product.

According to a second advantageous embodiment of the invention, the composition according to the invention is dedicated to making up the lips, and it is then more particularly a lipstick (in stick form) or a lip gloss (liquid lipstick).

According to one particular aspect, the invention relates to a process for making up and/or caring for the skin and/or the lips, comprising at least the application, to said skin and/or said lips, of the composition as defined above.

The present invention is illustrated in a nonlimiting manner in greater detail in the following examples.

### EXAMPLE 1

### Ureidopyrimidone-Functionalized Octyldodecanol of Structure

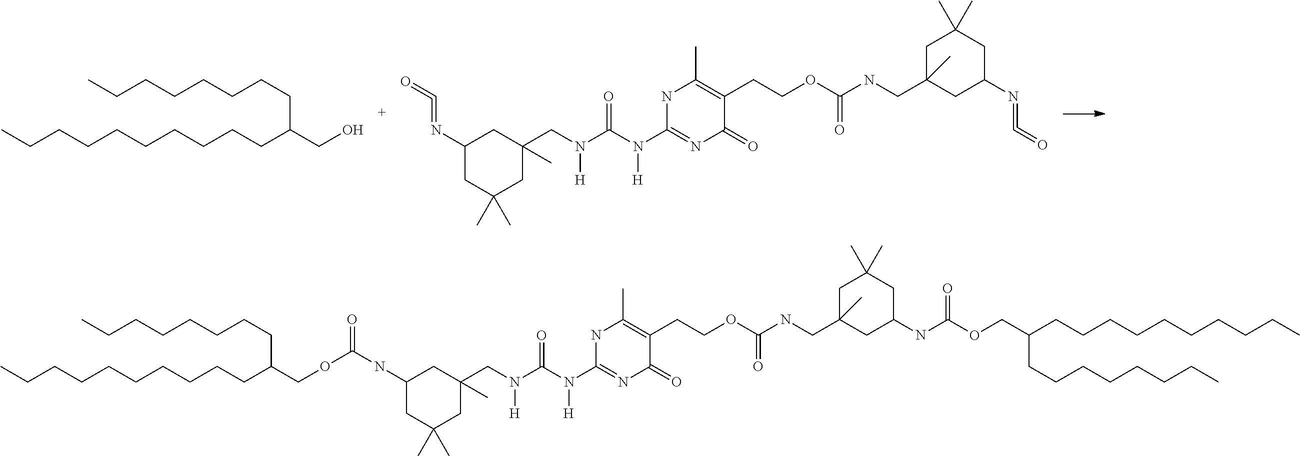

70 g of ureidopyrimidone diisocyanate are dissolved in methyltetrahydrofuran, under argon. 80.3 g of octyldodecanol in 100 ml of dichloromethane are added, under argon, followed by 15 microlitres of dibutyltin dilaurate (catalyst). The reaction mixture is refluxed until disappearance of the isocyanate peak (2250-2265 cm⁻¹) in IR spectrometry.

The excess octyldodecanol is eliminated by successive washing of the reaction medium with methanol, followed by three extractions and drying over MgSO₄. After evaporation of the organic phase, 103 g of a slightly yellow powder, characterized by ¹H NMR (structure complies), are obtained.

This powder can be carried in isododecane, for example at a concentration of 10% by weight; this concentration can range in particular up to 60% by weight in isododecane, which then results in a solution that is viscous but can still be handled. It is therefore noted that, functionalization with a ureidopyrimidone brings about a change from a liquid oil to a solid, which can be carried in isododecane at concentrations above 30%.

### EXAMPLE 2

### Ureidopyrimidone-Functionalized Octyldodecanol of Structure

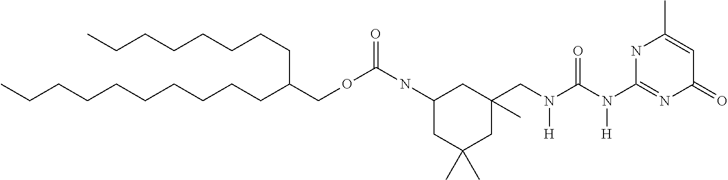 [SMILES: CCCCCCCCCCC(CCCCCCCC)COC(=O)NC1CC(C)(C)CC(C)(CNC(=O)Nc2nc(=O)cc(C)[nH]2)C1]

180 g of Jarcol I-20 (octyldodecanol) are run, at 50° C., into IPDI (isophorone diisocyanate, 1.1 eq.) in the presence of catalyst, with the exothermicity being controlled, under an inert atmosphere.

Stirring is maintained for 30 minutes at 50° C. 1.3 equivalents of methylisocytosine (MIC) and then 100 ml of propylene carbonate are added.

The temperature of the reaction medium is then increased to 140° C. and stirring is maintained for 1 hour at 140° C. The reaction is monitored by infrared spectroscopy, with monitoring of the decrease in the peak characteristic of the isocyanate function. The temperature is reduced to 70° C. and then 30 ml of ethanol are added and the resulting mixture is stirred for one hour. After addition of ethyl acetate, the medium is filtered through filter paper, the ethyl acetate is evaporated off and cyclohexane is added, and then washing is carried out 5 times with a mixture of NaCl-saturated water/ethanol (2V/1V). The organic phase is then dried over Na₂SO₄, filtered, and stripped with isododecane.

A solution at 50% of dry extract of the desired compound is then obtained.

### EXAMPLE 3

### Ureidopyrimidone-Functionalized Diisostearyl Malate

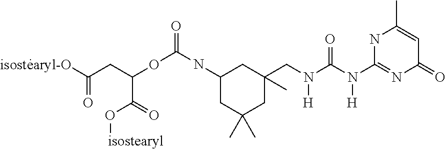

15 g (0.0234 mol) of diisostearyl malate are dried under reduced pressure, at 80° C., for 4 hours. 7.21 g (0.0117 mol) of ureidopyrimidone diisocyanate in solution in 60 ml of tetrahydrofuran, and 12 μl of dibutyltin dilaurate catalyst, are added. The mixture is heated at 95° C., under argon, for 26 hours (disappearance of the band characteristic of the isocyanates by IR spectroscopy). 20 ml of methyltetrahydrofuran are added to the reaction mixture, and filtration through celite is then carried out. After evaporation of the solvent and drying under reduced pressure, a pale yellow solid is obtained.

### EXAMPLE 4

### Ureidopyrimidone-Functionalized Diisostearyl Malate

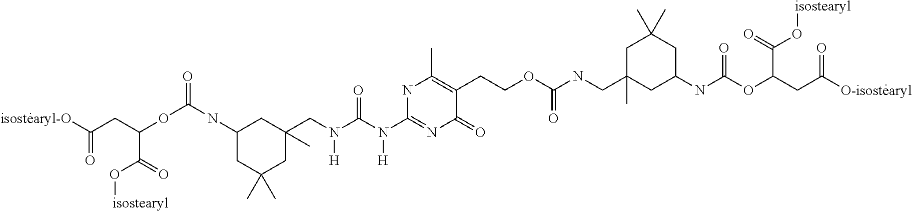

39 mg of catalyst (dibutyltin dilaurate) and 116.8 g of diisostearyl malate are mixed together, heated to 120° C. and dried under vacuum for 2 hours. The temperature is reduced to 60° C., under a controlled atmosphere, and 40.7 g of diisophorone diisocyanate are added, followed by stirring for 2 hours at 60° C. The temperature is then brought to 120° C., and then 15 ml of dry propylene carbonate and 15.6 g of 5-hydroxyethyl-6-methylisocytosine are added. The reaction mixture is heated to 140° C., and then left to stir at this temperature for 5 hours. Disappearance of the peak at 2250 cm⁻¹ corresponding to the disappearance of the isocyanate functions is observed. The temperature is then brought to 100° C. and 130 g of isododecane are then added; the solution is filtered and washed twice with ethanol. After stripping with isododecane, the desired compound at 50% of dry extract in isododecane is obtained.

### EXAMPLE 5

### Ureidopyrimidone-Functionalized Castor Oil

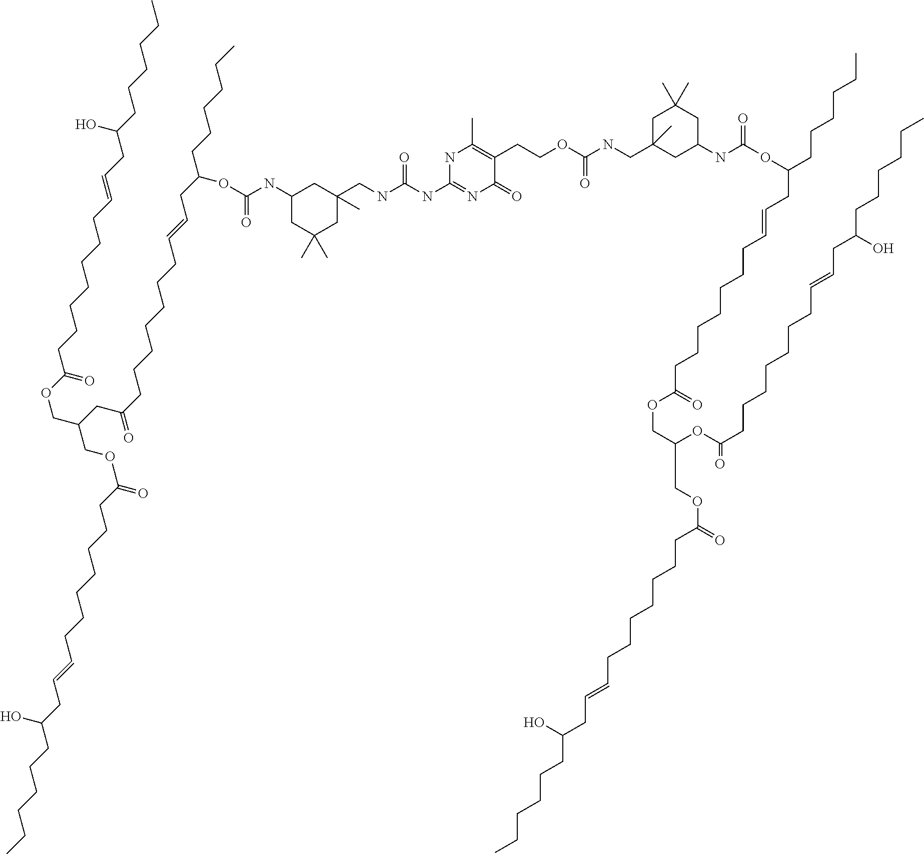

In a 250 ml round-bottomed flask, 15 g of castor oil (0.016 mol) are dried under reduced pressure at 80° C., for 2 hours. 4.91 g of ureidopyrimidone diisocyanate (0.008 mol) are solubilized in 60 ml of methyltetrahydrofuran. As soon as the vacuum is halted, the round-bottomed flask is placed under an inert atmosphere (argon). The ureidopyrimidone diisocyanate solution is rapidly run into the castor oil, and then the catalyst (dibutyltin dilaurate) is added. The reaction medium is heated at 90° C., with stirring, for 20 hours. The progression of the reaction is monitored by infrared spectrometry (isocyanate band at 2250-2280 cm⁻¹). At the end of the reaction, the residual isocyanates are neutralized by adding ethanol at 70° C. for 2 hours. The solvent is evaporated off and the resulting product is dried under reduced pressure, at 35° C. overnight.

A pale yellow solid gum corresponding to the desired product is obtained.

### EXAMPLE 6

### Ureidopyrimidone-Functionalized Castor Oil

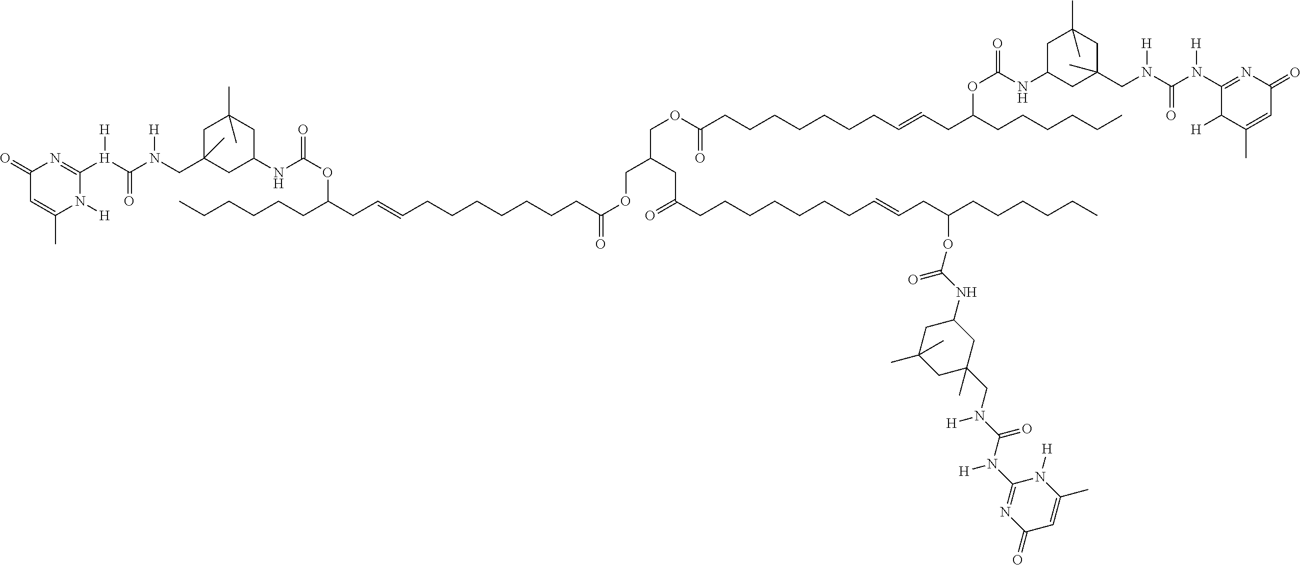

In a 250 ml reactor, under an inert atmosphere (argon), castor oil (16.3 g) and isophorone diisocyanate (0.04 mol) are mixed together. The reaction medium is diluted, with stirring, with 16 ml of butyl acetate. The catalyst (dibutyltin dilaurate) diluted in 2 ml of butyl acetate is added. The resulting mixture is heated at 40° C., with stirring, for 5 hours.

5.07 g of methylisocytosine powder (0.04 mol) are added and then rinsing is carried out with propylene carbonate (10 ml) and butyl acetate (5 ml). The reaction medium is heated at 140° C. for 2 h10. The reaction is monitored by IR spectroscopy and disappearance of the band characteristic of the isocyanates. Ethanol is added, in order to neutralize the residual isocyanates, at 70° C. for 3 hours and then overnight at ambient temperature. The reaction medium is diluted in methyltetrahydrofuran and filtered through celite and filter paper.

At the end of the reaction, the solvent is evaporated off and the resulting product is dried under reduced pressure, at 35° C. overnight.

A pale yellow solid corresponding to the desired product is obtained.

### EXAMPLE 7

### Ureidopyrimidone-Functionalized 2-Hexyldecanol

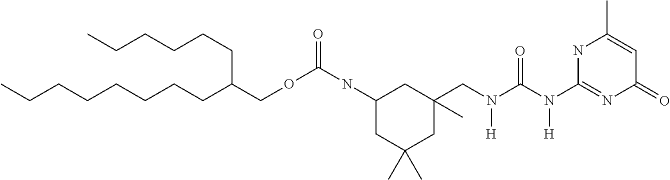 [SMILES: CCCCCCCCC(CCCCCC)COC(=O)NC1CC(C)(C)CC(C)(CNC(=O)Nc2nc(=O)cc(C)[nH]2)C1]

126.4 g of 2-hexyldecanol (Jarcol I-16) are heated at 60° C., under reduced pressure for 2 hours in order to dry the latter. After 2 hours, the oil is allowed to return to 20° C. under argon, and then added slowly, over 5 hours, to a mixture of 116 g of isophorone diisocyanate and 55 mg of DBTL catalyst at 50° C. At the end of the addition, the temperature of the reaction mixture is brought to 110° C., and then 90 ml of propylene carbonate and 78.4 g of 6-methylisocytosine are added, which results in a homogeneous white suspension. Stirring is maintained at 110° C. for two hours and the disappearance of the isocyanate is monitored by infrared spectroscopy. Disappearance of the peak is observed at 2250 cm⁻¹. In parallel, the disappearance of the amine originating from the isocytosine is monitored by quantitative determination of amines. At the end of the reaction, 500 g of isododecane are added, at 100° C., and a slightly cloudy, pale yellow solution is obtained. 300 ml of ethanol are added and stirring is maintained for 2 hours. After filtration through celite, the reaction mixture is stripped with isododecane at 80° C. in order to eliminate the alcohol and the propylene carbonate.

The desired product, carried in isododecane at 50% of dry extract, is finally obtained. The product is in particular characterized by HPLC and GPC (structure confirmed).

### EXAMPLE 8

### Ureidopyrimidone-Functionalized 2-Hexyldecanol

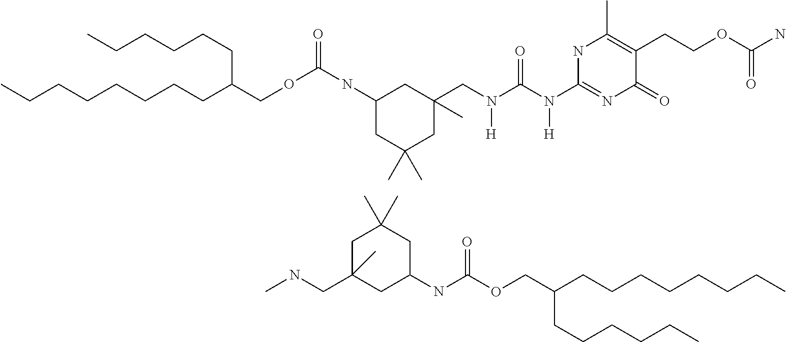

173.1 g of 2-hexyldecanol (Jarcol I-16) are heated at 60° C. under reduced pressure for 2 hours in order to dry the latter. After 2 hours, the oil is allowed to return to 50° C. under argon, and is then added slowly, over 5 hours, to a mixture of 158.7 g of isophorone diisocyanate and 77 mg of DBTL catalyst at 50° C. At the end of the addition, the temperature of the reaction mixture is brought to 110° C., and then 150 ml of propylene carbonate and 60.3 g of 5-hydroxyethyl-6-methylisocytosine are added, which results in a homogeneous white suspension. Stirring is maintained at 110° C. for five hours and the disappearance of the isocyanate is monitored by infrared spectroscopy. The disappearance of the peak is observed at 2250 cm⁻¹. At the end of the reaction, the temperature of the reaction medium is reduced to 100° C., and 780 g of isododecane are added; a slightly yellow, cloudy mixture is obtained. 100 ml of ethanol are added and stirring is maintained for 2 hours. After filtration through celite, the reaction mixture is stripped with isododecane at 80° C. in order to eliminate the alcohol and the propylene carbonate.

The desired product, carried in isododecane at 50% of dry extract, is finally obtained. The product is in particular characterized by HPLC and GPC (structure confirmed).

### EXAMPLE 9

### Ureidopyrimidone-Functionalized 2-Decyltetradecanol

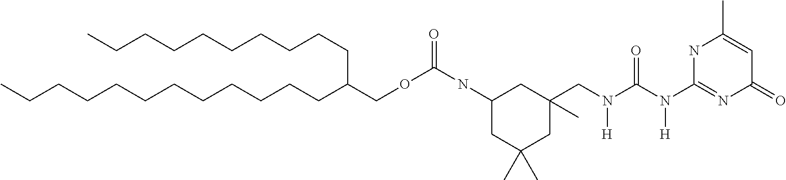 [SMILES: CCCCCCCCCCCCC(CCCCCCCCCC)COC(=O)NC1CC(C)(C)CC(C)(CNC(=O)Nc2nc(=O)cc(C)[nH]2)C1]

126 g of 2-decyltetradecanol (Jarcol I-24) are heated at 100° C. under reduced pressure for 4 hours in order to dry the latter. After 2 hours, the oil is added, over 4 hours, at 50° C. and under argon, to a mixture of 94.7 g of isophorone diisocyanate and of DBTL catalyst (qs). Monitoring by quantitative determination of isocyanate makes it possible to monitor the reaction; at half-equivalence, 126 g of propylene carbonate and 53.3 g of 6-methylisocytosine are added. Stirring and heating are maintained at 100° C. for 16 hours, and the disappearance of the isocyanate is monitored by infrared spectroscopy. The disappearance of the peak is observed at 2250 cm⁻¹. In parallel, the disappearance of the amine originating from the isocytosine is monitored by quantitative determination of amines. At the end of the reaction, the temperature is brought to 50° C., 100 ml of ethanol are added and stirring is maintained for 5 h. After filtration through celite and stripping with isododecane, the desired product, carried in isododecane at 50% of dry extract, is obtained. The product is in particular characterized by GPC and HPLC coupled to a mass spectrum.

### EXAMPLE 10

### Ureidopyrimidone-Functionalized Lauryl Alcohol

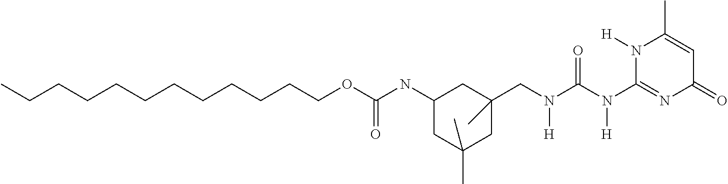

19.9 g of lauryl alcohol are heated under vacuum at 40° C. for 2 hours. 14 ml of isododecane are added and the resulting solution is added dropwise, over a period of 4 hours, to 23.7 g of isophorone diisocyanate, in the presence of dibutyltin dilaurate catalyst, under an inert atmosphere, at 50° C. Once the addition is complete, a solution of 16 g of 6-methylisocytosine in 15 ml of dry propylene carbonate is added and the mixture is heated to 140° C., followed by vigorous stirring for 5 hours, always under a controlled atmosphere.

The conversion of the isocyanate functions to urethane functions is monitored by IR spectroscopy, with disappearance of the band characteristic of the isocyanates at 2250 cm⁻¹. At the end of the reaction, the temperature is reduced to 50° C., and 100 ml of THF are added, followed by filtration through celite. The THF fraction is precipitated from pentane, followed by separation of the organic phase by settling out. The precipitate is then redissolved in THF and again precipitated from a methanol/water (1/1) mixture. The desired product is obtained after filtration and drying under vacuum.

### EXAMPLE 11

### Ureidopyrimidone-Functionalized Cetyl Alcohol

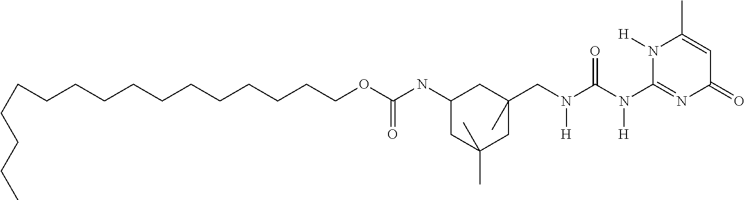

In a 250 ml reactor, under an inert atmosphere (argon), 25.05 g of cetyl alcohol are dissolved, with stirring, in 25 g of butyl acetate; the catalyst (dibutyltin dilaurate) is added to the medium. The medium is heated to 80° C. 24.1 g (0.08 mol) of compound of the following structure are added:

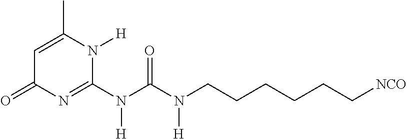

70 ml of butyl acetate are added. The reaction medium is heated at 100° C. for 48 hours. The reaction is monitored by IR spectroscopy and disappearance of the band characteristic of the isocyanates.

At the end of the reaction, the solvent is evaporated off and the resulting product is dried under reduced pressure, at 35° C. overnight.

### EXAMPLE 12

### Synthesis of a Polyalkene-Based Polymer (Polymer 1)

100 g of dihydroxylated hydrogenated 1,2-polybutadiene polymer (GI3000 from the company Nisso) are dried at 80° C., under reduced pressure, overnight. This polymer is dissolved in 400 ml of anhydrous toluene. 25 μl of catalyst (dibutyltin dilaurate) are added and the mixture is heated at 80° C., with stirring, until a homogeneous solution is obtained. 15 g of isocyanate-functionalized molecule having the following structure:

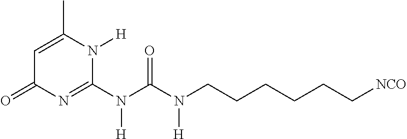

in solution in 300 ml of anhydrous toluene are added, under a controlled atmosphere at 40° C. The reaction mixture is heated to 100° C. and stirred at this temperature for 4 hours. The reaction is monitored by infrared spectroscopy, with monitoring of the complete disappearance of the peak characteristic of the isocyanates at 2260 cm⁻¹. At the end of the reaction, 100 ml of ethanol are added in order to eliminate all traces of residual isocyanate, and then the mixture is filtered after having added isododecane so as to make the solution less viscous. The polymer solution is then directly stripped with isododecane.

A solution of the final polymer in isododecane, at 21% of dry extract, is obtained; the polymer is characterized by GPC (Mn=6400 and polydispersity index=1.85) and ¹H NMR (spectrum in accordance with what is expected).

### EXAMPLE 13

### Synthesis of a Polyalkene-Based Polymer (Polymer 2)

### Synthesis of the Ureidopyrimidone-Difunctionalized GI2000 Polymer

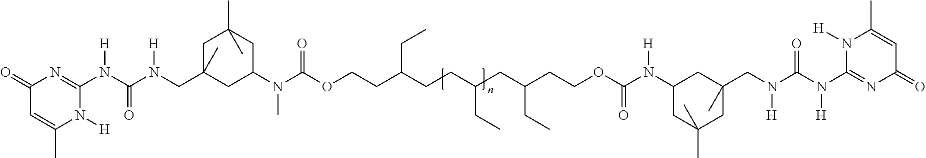

106.1 g of dihydroxylated hydrogenated 1,2-polybutadiene polymer (GI2000 from Nisso, Mn=3300 measured by GPC according to the protocol previously described) are heated in the presence of 22 mg of catalyst (dibutyltin dilaurate) at 80° C., under reduced pressure, for 2 hours. The temperature of the mixture is reduced to 20° C., under argon, followed by the addition of 10 ml of isododecane and 19.3 g of isophorone diisocyanate (IPDI). The mixture is stirred for 16 hours at 20° C., under a controlled atmosphere, and is then heated to 120° C., followed by the addition of 25 ml of propylene carbonate. 12 g of 6-methylisocytosine are added, resulting in a homogeneous white suspension. This suspension is heated to 140° C. and is stirred at this temperature for 6 hours. The reaction is monitored by infrared spectroscopy, until complete disappearance of the peak characteristic of the isocyanates (2250 cm⁻¹). The mixture is then brought back down to 30° C., and 400 ml of heptane, 200 ml of THF and 50 ml of ethanol are added thereto, before filtration through celite. The mixture is then stripped with isododecane.

In the end, a solution of the polymer in isododecane, at 25% of dry extract, is obtained; the polymer is characterized by GPC (Mn=7000 and polydispersity index=2.05).

### EXAMPLE 14

### Synthesis of a Polyalkene-Based Polymer (Polymer 3)

99 g of dihydroxylated hydrogenated 1,2-polybutadiene polymer (GI3000 from Nisso) are heated in the presence of 22 mg of catalyst (dibutyltin dilaurate) at 80° C., under reduced pressure, for 2 hours. The temperature of the mixture is brought down to 20° C., under argon, followed by the addition of 30 ml of isododecane and 11 g of isophorone diisocyanate (IPDI). The mixture is stirred for 16 hours at 20° C. under a controlled atmosphere, and is then heated to 120° C., followed by the addition of 25 ml of propylene carbonate. 8.1 g of 6-methylisocytosine are added, resulting in a homogeneous white suspension. This suspension is heated to 140° C. and is stirred at this temperature for 6 hours. The reaction is monitored by infrared spectroscopy, until complete disappearance of the peak characteristic of the isocyanates (2250 cm⁻¹). The mixture is then brought back down to 30° C., and 1 litre of heptane is added thereto, before filtration through cellite. The mixture is then stripped with isododecane.

In the end, a solution of the polymer in isododecane, at 20% of dry extract, is obtained; the polymer is characterized by GPC (Mn=4200 and polydispersity index=2.34).

### EXAMPLE 15

### Synthesis of a Polyalkene-Based Polymer (Polymer 4)

89 g of dihydroxylated hydrogenated 1,2-polybutadiene polymer (GI3000 from Nisso) are heated in the presence of 22 mg of catalyst (dibutyltin dilaurate) at 80° C., under reduced pressure, for 2 hours. The temperature of the mixture is brought down to 20° C., under argon, followed by the addition of 60 ml of isododecane and 11.6 g of 4,4′-dicyclohexylmethane diisocyanate. The mixture is stirred for 16 hours at 20° C., under a controlled atmosphere, and is then heated to 120° C., followed by the addition of 40 ml of propylene carbonate. 6.64 g of 6-methylisocytosine are added, resulting in a homogeneous white suspension. The suspension is heated to 140° C. and is stirred at this temperature for 8 hours. The reaction is monitored by infrared spectroscopy, until complete disappearance of the peak characteristic of the isocyanates (2250 cm⁻¹). The mixture is then brought back down to 30° C. and 250 ml of isododecane and 500 ml of heptane are added thereto, before filtration through celite. The mixture is then stripped with isododecane.

In the end, a solution ot the polymer in isododecane, at 22% of dry extract, is obtained; the polymer is characterized by GPC (Mn=10700 and polydispersity index=2.26).

### EXAMPLE 16

### Synthesis of a Polyalkene-Based Polymer (Polymer 5)

143.1 g of dihydroxylated hydrogenated 1,2-polybutadiene polymer (GI2000 from Nisso) are heated in the presence of 33 mg of catalyst (dibutyltin dilaurate) at 80° C., under reduced pressure, for 2 hours. The temperature of the mixture is brought down to 20° C., under argon, followed by the addition of 85 ml of isododecane and 30.8 g of 4,4′-dicyclohexylmethane diisocyanate. The mixture is stirred for 16 hours at 20° C., under a controlled atmosphere, and is then heated to 120° C., followed by the addition of 70 ml of propylene carbonate. 22.6 g of 6-methylisocytosine are added, resulting in a homogeneous white suspension. This suspension is heated to 140° C. and is stirred at this temperature for 8 hours. The reaction is monitored by infrared spectroscopy, until complete disappearance of the peak characteristic of the isocyanates (2250 cm⁻¹). The mixture is then brought back down to 20° C., and 700 ml of isododecane and 500 ml of heptane are added thereto, before filtration through celite. The mixture is then stripped with isododecane.

In the end, a solution ot the polymer in isododecane, at 20% of dry extract, is obtained; the polymer is characterized by GPC (Mn=8400 and polydispersity index=2.00).

### EXAMPLE 17

### Ureidopyrimidone-Functionalized Diisostearyl Malate

Preparation of the Supramolecular Compound Derived from an Oil: Ureidopyrimidone-Functionalized Diisostearyl Malate

150 g of diisostearyl malate were run, for 1 h20 at 50° C., into a solution of 57.4 g of isophorone diisocyanate and 38.18 g of methylisocytosine in the presence of the dibutyltin dilaurate catalyst, with the exothermicity being controlled, and under an inert atmosphere. Stirring was maintained for 55 minutes at 50° C. after the running-in, and then 50 ml of propylene carbonate were added. The temperature of the reaction medium was then increased to 140° C. with a contact time of 2 hours, with stirring. The temperature of the reaction medium was then reduced to 70° C., neutralization was carried out by adding 30 ml of ethanol and stirring was continued for one hour.

After the addition of 780 ml of ethyl acetate, the medium was filtered through celite. After evaporation of the ethyl acetate, 400 ml of cyclohexane were added to the reaction medium and the mixture was washed twice with a mixture of H₂O/EtOH (2 v/1 v) saturated with NaCl. The organic phase was then stripped with isododecane, until a viscous liquid was obtained, corresponding to the desired molecule at 50% of dry extract. This dry extract can optionally be modified by further addition of isododecane to the medium, according to the requirements of the formulation.

### EXAMPLE 18

### Ureidopyrimidone-Functionalized Jarcol 24 (J24)

200 g of Jarcol I-24 are run, at 50° C., into IPDI (1.1 eq. IPDI) in the presence of the catalyst, with the exothermicity being controlled, and under an inert atmosphere. Stirring is maintained for 30 minutes at 50° C. after the running-in. 1.3 equivalents of methylisocytosine (MIC) are then added to the mixture, followed by the addition of 100 ml of propylene carbonate. The temperature of the reaction medium is then increased to 140° C., with a contact time of 1 h at 140° C. The disappearance of the isocyanate functions is monitored by infrared spectroscopy, and then the temperature of the medium is reduced to 70° C., followed by the addition of 30 ml of ethanol and stirring for 1 h. After the addition of ethyl acetate, the medium is filtered through filter paper. After evaporation of the ethyl acetate, cyclohexane is added, followed by 5 washes with a mixture of NaCl-saturated water/ethanol (2V/1V). The organic phase is then dried over Na₂SO₄, filtered, and stripped with isododecane. A solution at 50% of dry extract of ureidopyrimidone-functionalized oil is then obtained.

### EXAMPLE 19

### A/ Gloss Measured Using a Glossmeter on a Dry Deposit of Polymer

A coat 200 μm thick of the compound/mixture to be tested, at 10% in isododecane, is spread onto a PA-2810 Byko-Chart contrast card using an automatic spreader. The coat covers at least the black background of the card. The deposit is left to dry for 24 hours at a temperature of 23° C., and then the gloss at 20° is then measured on the black background using a Byk Gardner Micro-Tri-Gloss glossmeter. A measurement at 20° higher than 50 is equivalent to a gloss considered to be acceptable, and if the measurement is greater than 60, the gloss is considered to be very satisfactory.

### B/ Measurement of Wear Resistance

A coat 200 μm thick of the compound/mixture to be tested, at 10% in isododecane, is spread onto a PA-2810 Byko-Chart contrast card using an automatic spreader. The coat covers at least the black background of the card. The deposit is left to dry for 24 hours at a temperature of 23° C. The wear resistance of the film formed is evaluated using a tribometer of pin-on-disc type; the substrate+film sample is moved and is in contact with a rubbing device which is a steel ball between 5 and mm in diameter. The load is between 0.25 and 3 N, and the movement speed is between 10 and 50 mm/s. The number of passes of the rubbing device, in the same place, necessary in order to completely wear the film is measured. The higher this number, the greater the wear resistance of the film.

The following results are obtained:

| | Gloss | |
|---|---|---|
| | at 20° C. | Wear resistance |
| Polymer 2 (example 13) | 60 | between 50 and 100 |
| | | revolutions |
| Functionalized lauryl alcohol | 0 | Less than 20 revolutions |
| (example 10) | | |
| Polymer 2 (ex. 13) + lauryl alcohol | 60 | Greater than 500 |
| (ex. 10): 80/20 by weight | | revolutions |
| Functionalized cetyl alcohol | 80 | Less than 20 revolutions |
| (example 11) | | |
| Polymer 2 (ex. 13) + cetyl alcohol | 70 | Greater than 200 |
| (ex. 11): 80/20 by weight | | revolutions |

### EXAMPLE 20

Four liquid lip makeup formulations having the following composition (% by weight) were prepared. Formulations 1 to 4 illustrate the invention and formulation 5 is a comparative composition outside the invention.

| | | | | | Formulation 5 |
|---|---|---|---|---|---|
| | Formulation | Formulation | Formulation | Formulation | (outside |
| | 1 (invention) | 2 (invention) | 3 (invention) | 4 (invention) | the invention) |
| Diisostearyl malate- | | | | 15% | |
| ureidopyrimidone | | | | (7.5% | |
| (ex. 17) at 50% in a | | | | active | |
| 95/5 isododecane/ethanol | | | | material | |
| mixture | | | | (AM)) | |
| Ureidopyrimidone- | | 15% | | | |
| functionalized Jarcol | | (7.5% AM) | | | |
| 20 (ex. 2) at | | | | | |
| 50% in a 95/5 iso- | | | | | |
| dodecane/ethanol | | | | | |
| mixture | | | | | |
| Ureidopyrimidone- | 15% | | | | |
| functionalized Jarcol | (7.5% AM) | | | | |
| 24 (ex. 18) at | | | | | |
| 50% in a 95/5 iso- | | | | | |
| dodecane/ethanol | | | | | |
| mixture | | | | | |
| Ureidopyrimidone- | | | 15% | | |
| functionalized Jarcol | | | (7.5% AM) | | |
| 16 (ex. 7) at | | | | | |
| 50% in a 95/5 iso- | | | | | |
| dodecane/ethanol | | | | | |
| mixture | | | | | |
| Solution of | 54% | 54% | 54% | 54% | 54% |
| ureidopyrimidone- | (13.5% AM) | (13.5% AM) | (13.5% AM) | (13.5% AM) | (13.5% AM) |
| difunctionalized | | | | | |
| GI2000 supramolecular | | | | | |
| polymer at | | | | | |
| 25% in isododecane | | | | | |
| (example 13) | | | | | |
| Phenyl silicones | 24.61 | 24.61 | 24.61 | 24.61 | 39.61 |
| 20 cst (phenyl tri- | | | | | |
| methicone) DC556 | | | | | |
| DC Red 7 | 0.59 | 0.59 | 0.59 | 0.59 | 0.59 |
| Titanium oxide | 2.74 | 2.74 | 2.74 | 2.74 | 2.74 |
| Blue 1 lake | 0.16 | 0.16 | 0.16 | 0.16 | 0.16 |
| Yellow lake 6 | 2.58 | 2.58 | 2.58 | 2.58 | 2.58 |
| Black iron oxide | 0.32 | 0.32 | 0.32 | 0.32 | 0.32 |
| Total | 100 | 100 | 100 | 100 | 100 |

### Preparation of the Compositions

The pigments were milled in a part of the silicone oil by carrying out 3 passes of the mixture on the triple-roll mill. The supramolecular polymer and/or the functionalized oils according to the invention were mixed with the rest of the silicone oil and the pigment mill base in a beaker or heating vessel. The mixture was then stirred with a Rayneri stirrer until it was homogeneous.

The formulations were poured into small pots which are leaktight with regard to the isododecane.

### Evaluation of the Compositions:

The staying power, and in particular the oil resistance, of the deposits produced with compositions 1 to 5 was evaluated visually, along with the residual gloss of the makeup deposits after having carried out the oil-resistance evaluation test.

A sample of each of the compositions was spread, at ambient temperature (25° C.) over the surface of a polystyrene sheet. After drying of the deposits for 24 h at ambient temperature, olive oil was applied to the entire surface of the polystyrene sheet, so as to completely cover each of the deposits produced with the compositions to be evaluated.

For each of the compositions, a finger was applied to the deposit thus covered and a rotation was then performed while maintaining a constant pressure for a period of 30 seconds.

The surface was then wiped 3 times with a paper handkerchief.

Finally, the appearance of the residual deposit on the sheet after wiping was evaluated.

The following results were obtained:

| | Formulation | Formulation | Formulation | Formulation | Formulation |
|---|---|---|---|---|---|
| | 1 according | 2 according | 3 according | 4 according | 5 outside of |
| | to the invention | to the invention | to the invention | to the invention | the invention |
| Appearance | Deposit of | Deposit of | Deposit of | Deposit of | Deposit very |
| of the deposit | uniform | uniform | uniform | uniform | uneven, |
| | thickness | thickness | thickness | thickness | fragmented |
| | and colour | and colour | and colour | and colour | (having dis- |
| | (good colour | (good colour | (good colour | (good colour | appeared on |
| | fastness), | fastness), | fastness), | fastness), | some areas), |
| | and | and | and | and | poor |
| | very glossy | moderately | moderately | moderately | colour fastness, |
| | | glossy | glossy | glossy | glossy |
| | | | | | where there |
| | | | | | is still deposit |

It emerges from these results that the compositions in accordance with the invention make it possible to obtain a makeup deposit which exhibits good colour fastness, especially with respect to grease and in particular to oils, and good gloss fastness after drying at ambient temperature, in particular with regard to the deposit produced with comparative composition 5.

### EXAMPLE 21

Four foundation formulations according to the invention (formulations 1 to 4) and one comparative foundation formulation outside the invention (formula 5) were prepared, comprising (% by weight, AM=active material):

| | | Formu- | Formu- | Formu- | Formu- | Formu- |
|---|---|---|---|---|---|---|
| | | lation 1 | lation 2 | lation 3 | lation 4 | lation 5 |
| A1 | Solution of | 48% | 48% | 48% | 48% | 48% |
| | ureidopyrimidone- | (12% | (12% | (12% | (12% | (12% |
| | difunctionalized | AM) | AM) | AM) | AM) | AM) |
| | GI2000 supra- | | | | | |
| | molecular polymer | | | | | |
| | at 25% in | | | | | |
| | isododecane | | | | | |
| | (example 13) | | | | | |
| | Diisostearyl malate- | 10% | — | — | — | — |
| | ureidopyrimidone | (5% | | | | |
| | (ex. 17) at 50% | AM) | | | | |
| | in isododecane | | | | | |
| | Ureidopyrimidone- | — | 10% | — | — | — |
| | functionalized | | (5% | | | |
| | Jarcol 16 (ex. 7) | | AM) | | | |
| | at 50% in | | | | | |
| | isododecane | | | | | |
| | Ureidopyrimidone- | — | — | 10% | — | — |
| | functionalized | | | (5% | | |
| | Jarcol 20 (ex. 2) | | | AM) | | |
| | at 50% in | | | | | |
| | isododecane | | | | | |
| | Ureidopyrimidone- | — | — | — | 10% | — |
| | functionalized | | | | (5% | |
| | Jarcol 24 (ex. 18) | | | | AM) | |
| | at 50% in | | | | | |
| | isododecane | | | | | |
| | Isododecane | Qs | Qs | Qs | Qs | Qs |
| | | 100% | 100% | 100% | 100% | 100% |
| A2 | Isododecane | 2.5% | 2.5% | 2.5% | 2.5% | 2.5% |
| | Pigments | 10% | 10% | 10% | 10% | 10% |
| B | Silica micro- | 2% | 2% | 2% | 2% | 2% |
| | spheres (Miyoshi | | | | | |
| | Kasei, Sun- | | | | | |
| | sphere H-33, | | | | | |
| | AGC Si-TECH) | | | | | |

### Procedure:

The constituents of phase A2 were weighed out. The mixture was passed through a triple-roll mill. The constituents of phase A1 were weighed out into the main beaker and placed in a Rayneri stirrer. Phase A2 was then added. After stirring for minutes, phase B was incorporated.

The formulations 1, 2, 3 and 4 according to the invention exhibit a significantly improved application to the skin compared with the control composition 5. This is because their texture is more fluid; they spread better on the skin.

The result obtained in the end is also more cosmetic and less tacky.

The formulations can be classified according to their performance levels in meeting the following criteria (facilitated application and reduced tack): Formulation 5<formulation 1<formulation 2<formulation 3<formulation 4.

1. Cosmetic composition comprising, in a cosmetically acceptable medium:
a) at least one supramolecular oil (compound A) which can be obtained by reaction between:
an oil bearing at least one nucleophilic reactive function, in particular chosen from OH and NH₂, and
a joining group capable of establishing hydrogen bonds with one or more partner joining groups, each pairing of a joining group involving at least 4 hydrogen bonds, said joining group bearing at least one reactive function capable of reacting with the reactive function borne by the oil, in particular chosen from isocyanate, acid and imidazole, said joining group also comprising at least one unit of formula (Ia) or (Ib): 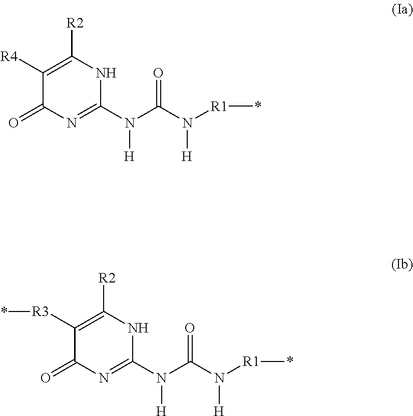
in which:
R1 and R3, which may be identical or different, represent a divalent carbon-based radical chosen from (i) a linear or branched C₁-C₃₂ alkyl group, (ii) a C₄-C₁₆ cycloalkyl group and (iii) a C₄-C₁₆ aryl group; said groups optionally comprising 1 to 8 heteroatoms chosen from O, N, S, F, Si and P; and/or optionally being substituted with an ester or amide function or with a C₁-C₁₂ alkyl radical; or a mixture of these groups;
R2 and R4, independently of one another, represent a hydrogen atom or a linear, branched or cyclic, saturated or unsaturated, optionally aromatic, C₁-C₃₂ carbon-based, in particular hydrocarbon-based (alkyl), radical which can comprise one or more heteroatoms chosen from O, N, S, F, Si and P; and
b) at least one polyalkene-based supramolecular polymer (compound B) which can result from the reaction, in particular by condensation, of at least one polyalkene polymer functionalized with at least one reactive group, with at least one joining group functionalized with at least one reactive group capable of reacting with the reactive group(s) borne by the functionalized polyalkene polymer, said joining group being capable of forming at least 3 H (hydrogen) bonds, preferably at least 4 H bonds, preferentially 4 H bonds.

2. Composition according to claim 1, in which the oil bearing at least one nucleophilic reactive function has a molar mass (Mw) of between 150 and 6000, in particular of between 170 and 4000, or even between 180 and 2000, preferentially between 200 and 1500, and even better still between 220 and 800 g/mol.

3. Composition according to claim 1, in which the oil bearing at least one nucleophilic reactive function is chosen, alone or as a mixture, from:
(i) linear, branched or cyclic, saturated or unsaturated, fatty alcohols comprising 6 to 50 carbon atoms, and comprising one or more OH, optionally comprising one or more NH₂;
(ii) esters and ethers bearing at least one free OH group, and in particular partial esters and ethers of a polyol, and hydroxylated carboxylic acid esters;
(iii) hydroxylated natural and modified natural plant oils.

4. Composition according to claim 1, in which the oil bearing at least one nucleophilic reactive function is chosen, alone or as a mixture, from:
linear or branched, saturated or unsaturated, C₆-C₅₀, especially C₆-C₃₂, in particular C₈-C₂₈, monoalcohols, and in particular isostearyl alcohol, cetyl alcohol, oleyl alcohol, isopalmitoyl alcohol, lauryl alcohol, 2-butyloctanol, 2-hexyldecanol, 2-octyldecanol, 2-octyldodecanol, 2-octyltetradecanol, 2-decyltetradecanol, 2-dodecylhexadecanol; and/or
esters between a hydroxylated dicarboxylic acid and monoalcohols, and in particular malic acid esters, and especially C₄-C₄₀ alkyl malates, such as 2-diethylhexyl malate, diisostearyl malate or 2-dioctyldodecyl malate;
hydrogenated or nonhydrogenated castor oil, and also derivatives thereof; hydroxylated modified soybean oil.

5. Composition according to claim 1, in which the oil bearing at least one nucleophilic reactive function is chosen from 2-octyldodecanol, diisostearyl malate, 2-butyloctanol, 2-hexyldecanol, 2-decyltetradecanol; hydrogenated or nonhydrogenated castor oil; hydroxylated modified soybean oil, and mixtures thereof.

6. Composition according to claim 1, in which, in the joining group of formula (Ia) or (Ib),
R1 represents -isophorone-, —(CH₂)₆— or 4,4′-methylenebiscyclohexylene; and/or
R2 represents H, CH₃, ethyl, C₁₃H₂₇, C₇H₁₅, phenyl, isopropyl, isobutyl, n-butyl, tert-butyl, n-propyl, or else —CH(C₂H₅)(C₄H₉); and/or
R4=H; and/or
R3 has the structure:

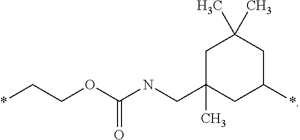

7. Composition according to claim 1, in which, in formula (Ia):
R1=-isophorone-, R2=methyl and R4=H, or
R1=—(CH₂)₆—, R2=methyl and R4=H, or
R1=—(CH₂)₆—, R2=isopropyl and R4=H, or
R1=4,4′-methylenebiscyclohexylene, R2=methyl and R4=H, or
in formula (Ib), R1=-isophorone-, R2=methyl and R3=—(CH₂)₂OCO—NH-isophorone-.

8. Composition according to claim 1, in which compound A corresponds to one of the following structures:
ureidopyrimidone-functionalized octyldodecanol of structures: 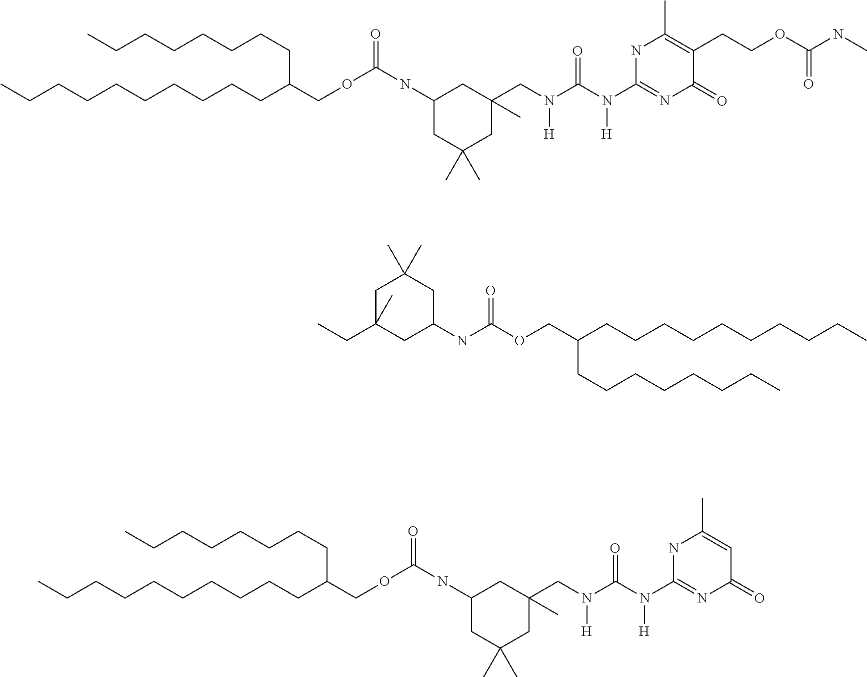
ureidopyrimidone-functionalized diisostearyl malate of structures: 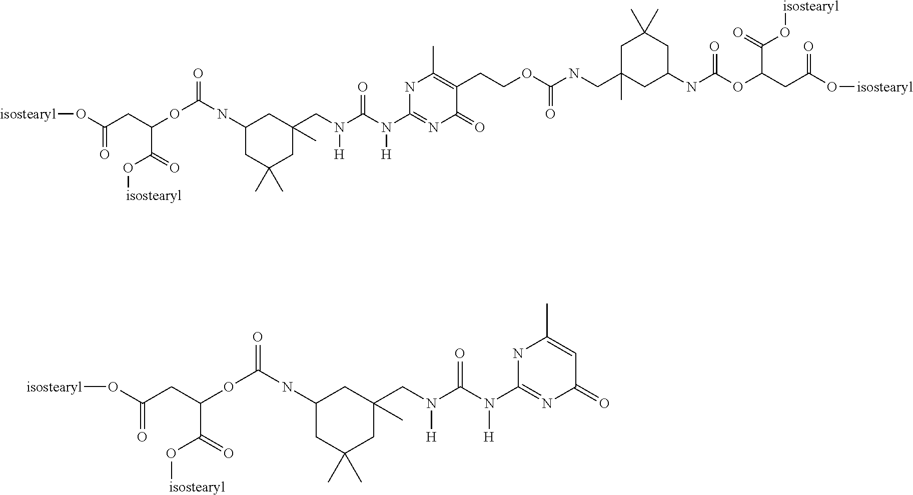
ureidopyrimidone-functionalized castor oil of structures: 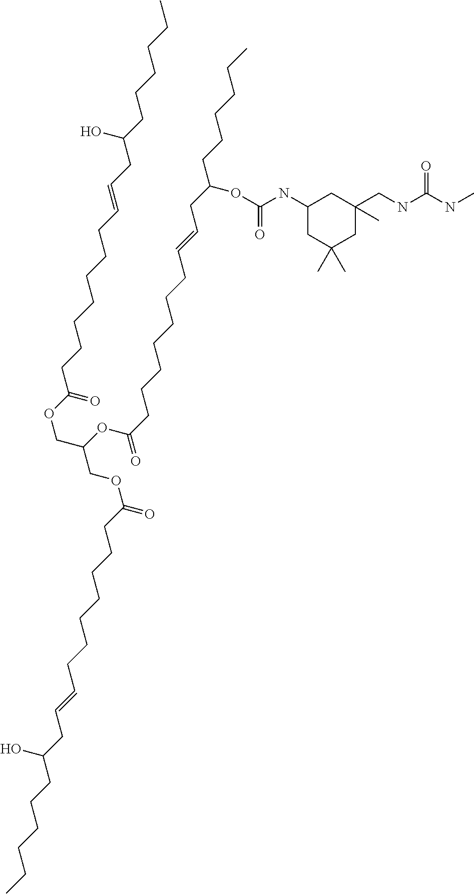 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(CCCCCC)OC(=O)NC1CC(C)(C)CC(C)(CNC(=O)NC)C1] 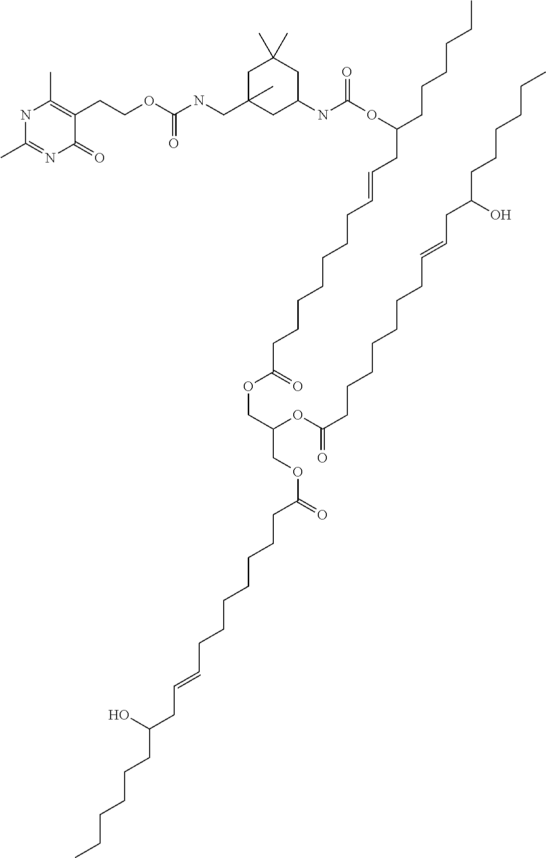
ureidopyrimidone-functionalized 2-hexyldecanol of structures: 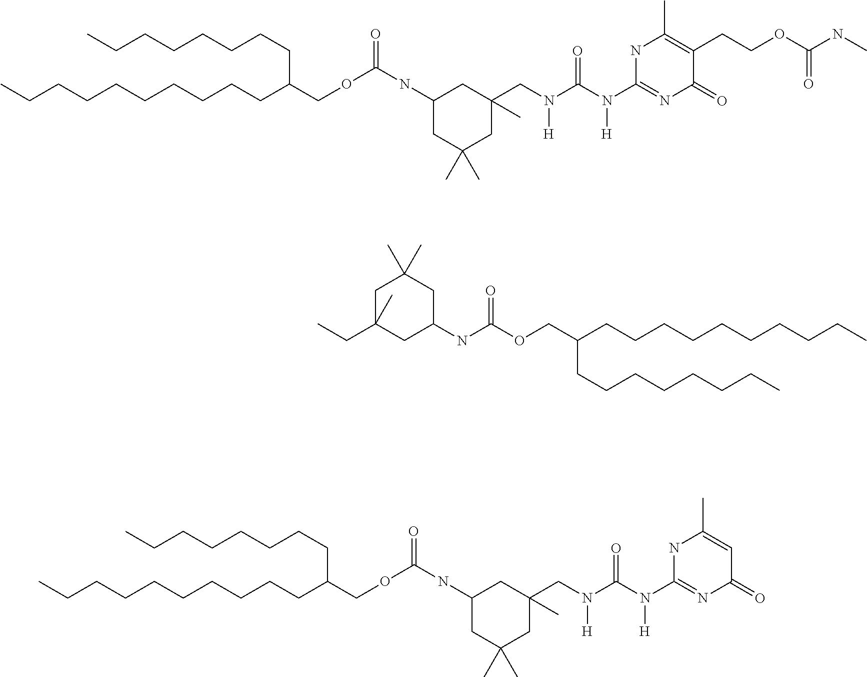
ureidopyrimidone-functionalized 2-decyltetradecanol of structures: 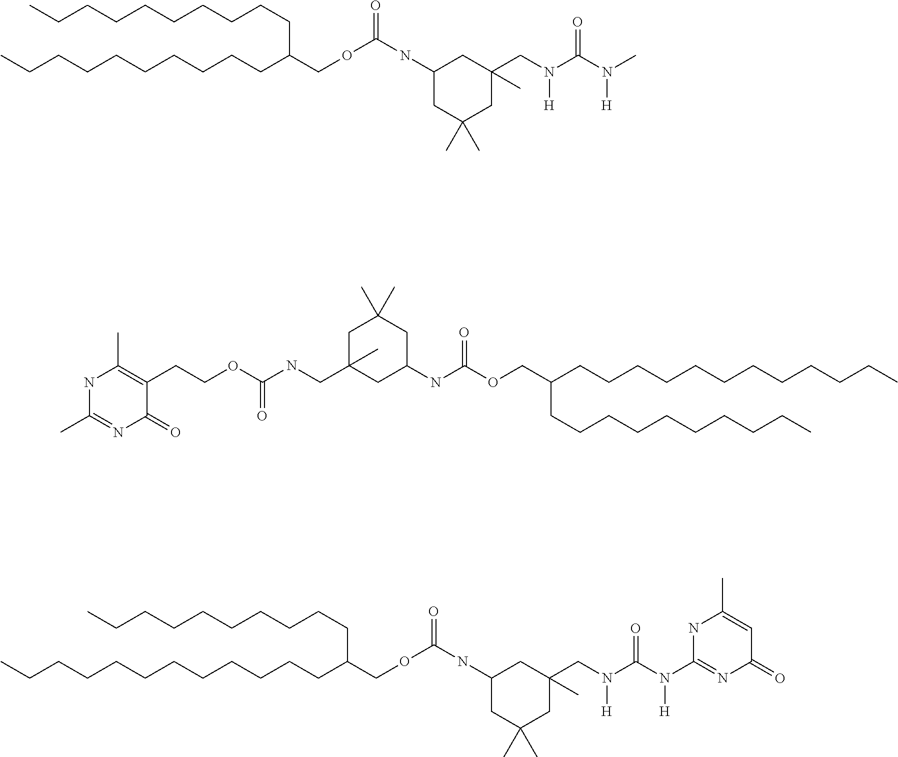
ureidopyrimidone-functionalized lauryl alcohol of structure: 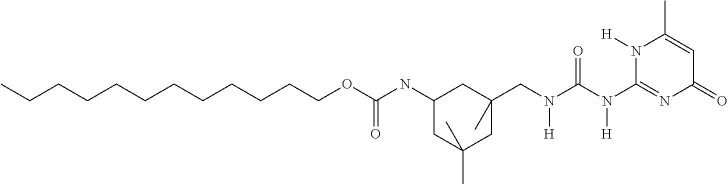
ureidopyrimidone-functionalized cetyl alcohol of structure:

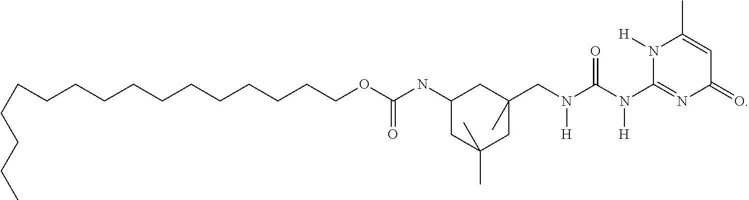

9. Composition according to claim 1, in which compound A, alone or as a mixture, is present in an amount of between 0.5% and 99% by weight, preferably between 0.5% and 50% by weight, in particular between 1% and 40% by weight, or even between 1.5% and 20% by weight, and better still between 2% and 15% by weight, relative to the weight of the final cosmetic composition.

10. Composition according to claim 1, in which the funcitonalized polyalkene polymer is of formula HX—P—X′H in which:
XH and X′H are reactive functions, with X and X′, which may be identical or different, chosen from O, S, NH, NCO or NRₐ, Rₐ representing a C₁-C₆ alkyl group; preferably, X and/or X′ denote O; preferentially, X and X′ denote O;
P represents a homopolymer or copolymer which can be obtained by polymerization of one or more linear, cyclic and/or branched, monounsaturated or polyunsaturated, C₂-C₁₀, preferably C₂-C₄, alkenes; P preferably represents a homopolymer or copolymer which can be obtained by polymerization of one or more linear or branched, monounsaturated C₂-C₄ alkenes.

11. Composition according to claim 10, in which P represents a polymer chosen from a polyethylene, a polybutylene, a polybutadiene (such as a 1,4-polybutadiene or a 1,2-polybutadiene), a polyisoprene, a poly(1,3-pentadiene), a polyisobutylene, and copolymers thereof, and in particular a poly(ethylene/butylene).

12. Composition according to claim 1, in which the functionalized polyalkene polymer is chosen from polydienes, which are preferably hydrogenated, comprising hydroxyl functions, preferably comprising hydroxyl ends, and polyolefins comprising hydroxyl ends; and in particular from polybutadiene, polyisoprene and poly(1,3-pentadiene) homopolymers and copolymers.

13. Composition according to claim 1, in which the functionalized polyalkene polymer is a dihydroxylated hydrogenated 1,2-polybutadine homopolymer, in particular those represented schematically by the following formula: 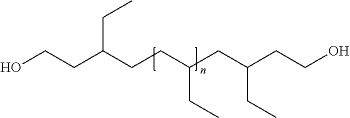
with n preferably between 14 and 105, better still between 20 and 85.

14. Composition according to claim 1, in which the functionalized joining group capable of reacting with the functionalized polyalkene polymer is of formula (III): 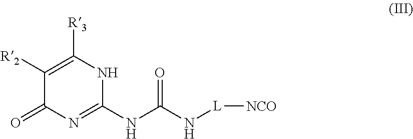
in which:
L is a single bond or a linear, cyclic and/or branched, saturated or unsaturated, or even aromatic, C₁-C₂₀ divalent carbon-based (alkylene) group, optionally comprising 1 to 4 N and/or O heteroatoms;
R′₂ represents a single bond, a divalent group of C₁-C₆ alkylene type, or a monovalent group chosen from a hydrogen atom or a linear, branched and/or cyclic, saturated or unsaturated, optionally aromatic, C₁-C₃₀ monovalent hydrocarbon-based group which can contain one or more heteroatoms such as O, S or N;
R′₃ represents a hydrogen atom or a linear, branched and/or cyclic, saturated or unsaturated, optionally aromatic, C₁-C₃₀ hydrocarbon-based group which can contain one or more heteroatoms such as O, S or N.

15. Composition according to claim 1, in which the functionalized joining group capable of reacting with the functionalized polyalkene polymer is of formula (IV): 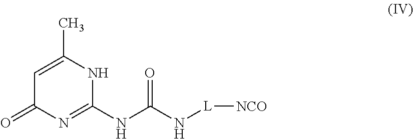
in which L is a single bond or a linear, cyclic and/or branched, saturated or unsaturated, or even aromatic, C₁-C₂₀ divalent carbon-based (alkylene) group, optionally comprising 1 to 4 N and/or O heteroatoms, in particular in the form of an NO₂ substituent, and in particular a phenylene; 1,4-nitrophenyl; 1,2-ethylene; 1,6-hexylene; 1,4-butylene; 1,6-(2,4,4-trimethylhexylene); 1,4-(4-methylpentylene); 1,5-(5-methylhexylene); 1,6-(6-methylheptylene); 1,5-(2,2,5-trimethylhexylene); 1,7-(3,7-dimethyloctylene); -isophorone-; 4,4′-methylenebiscyclohexylene; tolylene; 2-methyl-1,3-phenylene; 4-methyl-1,3-phenylene or 4,4-bisphenylenemethylene group; preferentially, L is -isophorone-; —(CH₂)₂—; —(CH₂)₆—; —CH₂CH(CH₃)—CH₂—C(CH₃)₂—CH₂—CH₂; 4,4′-methylenebiscyclohexylene or 2-methyl-1,3-phenylene; and better still isophorone.

16. Composition according to claim 1, in which the supramolecular polymer corresponds to the formula: 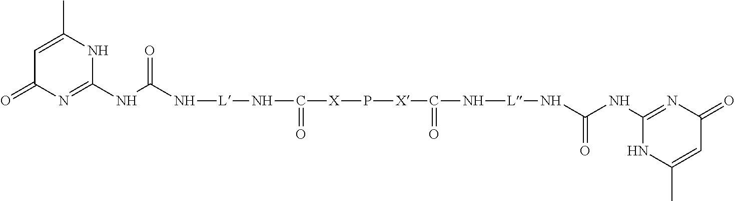
in which:
L′ and L″ are, independently of one another, a linear, cyclic and/or branched, saturated or unsaturated, or even aromatic, C₁-C₂₀ divalent carbon-based (alkylene) group, optionally comprising 1 to 4 N and/or O heteroatoms;
X═X′═O, and
P represents a homopolymer or copolymer which can be obtained by polymerization of one or more linear, cyclic and/or branched, monounsaturated or polyunsaturated, C₂-C₁₀ alkenes.

17. Composition according to claim 1, in which the supramolecular polymer is of formula: 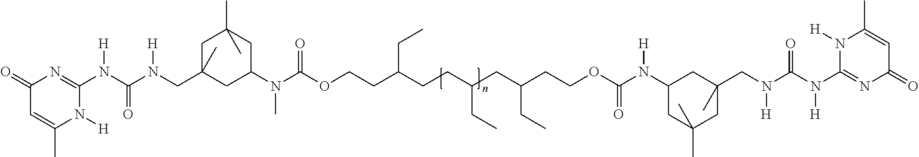
the value of n being such that the number-average molecular weight (Mn) of said polymer is between 1000 and 8000, in particular between 1200 and 5000, or even between 1500 and 4500, and even better still between 2000 and 4000.

18. Composition according to claim 1, in which the supramolecular polymer is present in an amount of between 0.1% and 99% by weight, preferably between 1% and 80% by weight, in particular between 2% and 70% by weight, or even between 3% and 60% by weight, and even better still between 4% and 50% by weight, preferentially 5% to 40% by weight of dry matter, relative to the weight of the final cosmetic composition.

19. Composition according to claim 1, in which the cosmetically acceptable medium comprises at least one constituent chosen from volatile or non-volatile, carbon-based, hydrocarbon-based and/or silicone oils and/or solvents of mineral, animal, plant or synthetic origin; pigments, fillers, pearlescent agents and glitter flakes, liposoluble or water-soluble dyes; water, hydrophilic solvents, waxes, pasty fatty substances, organopolysiloxane elastomers, antioxidants, fragrances, essential oils, preservatives, cosmetic active agents, moisturizers, vitamins, ceramides, sunscreens, surfactants, gelling agents, thickeners, spreading agents, wetting agents, dispersants, antifoams, neutralizing agents, stabilizers, polymers, and in particular film-forming polymers, and mixtures thereof.

20. Composition according to claim 1, in the form of a care and/or makeup product for the skin of the body or of the face, the lips, the eyelashes, the eyebrows, the hair or the nails; an anti-sun or self-tanning product; a hair product; they are advantageously in the form of a makeup composition, in particular a mascara, eyeliner, lipstick, lipgloss, face powder, eyeshadow, foundation, nail varnish or hair mascara composition.

21. Cosmetic treatment process for keratin fibres, in particular the skin of the body or of the face, the lips, the nails, the eyelashes and/or the hair, comprising the application, to said materials, of a cosmetic composition as claimed in claim 1.
